(19) United States
(12) Patent Application Publication
(10) Pub. No.: US 2012/0220513 A1
(43) Pub. Date: Aug. 30, 2012

(54) Polypeptides Having Detergency Enhancing Effect

(75) Inventors: Marie Allesen-Holm, Hilleroed (DK); Lars Anderson, Malmoe (SE); Kirk Matthew Schnorr, Holte (DK); Nikolaj Spodsberg, Bagsvaerd (DK); Paul Harris, Carnation, WA (US)

(73) Assignee: NOVOZYMES A/S, Bagsvaerd (DK)

(21) Appl. No.: 13/504,481

(22) PCT Filed: Dec. 28, 2010
(86) PCT No.: PCT/EP2010/070795

§ 371 Date: Apr. 26, 2012

Related U.S. Application Data
(60) Provisional application No. 61/290,703, filed on Dec. 29, 2009.

(51) Int. Cl. C11D 3/386; C11D 17/06; C11D 17/00; C12N 9/24; C07H 21/04

(52) U.S. Cl. 510/300; 435/200; 536/23.2; 510/392; 510/320; 510/515; 510/530; 510/393; 510/321

(57) ABSTRACT

The present invention relates to the use of glycosyl hydrolase family 61 polypeptides as enhancers of enzyme benefits in detergents as well as a detergent composition comprising glycosyl hydrolase family 61 polypeptides in combination with detergency enzymes.

### REFERENCE TO SEQUENCE LISTING

This application contains a Sequence Listing in computer readable form. The computer readable form is incorporated herein by reference.

### FIELD OF THE INVENTION

The present invention relates to the use of glycosyl hydrolase family 61 polypeptides as enhancers of enzyme benefits in detergents as well as a detergent composition comprising glycosyl hydrolase family 61 polypeptides in combination with detergency enzymes. The invention also relates to a process of washing a fabric or hard surface using the GH61 polypeptides in combination with an enzyme and/or with the detergents of the present invention. A further aspect of the invention relates to isolated glycosyl hydrolase family 61 polypeptides and isolated polynucleotides encoding the polypeptides as well as to vectors and host cells comprising the polynucleotides and methods of producing the polypeptides.

### BACKGROUND OF THE INVENTION

The addition of enzymes to both powder and liquid detergent formulations for both laundry and automatic dishwashing applications is common. The enzymes aid the stain removal from the objects (e.g. fabrics and hard surfaces) they are applied to by acting on specific components in the stains, such as proteins, starch, lipid, pectin and hemicellulose. Additionally, some enzymes provide additional benefits such as anti-pilling, fabric-softness, colour clarification, particulate soil removal, soil anti-redeposition and/or dye transfer inhibition.

The benefit of enzymes in detergency is constantly being explored. The addition of enzymes for example allow for decreased washing temperatures, and more recently there is a focus on whether enzymes can substitute some of the conventional detergent ingredients such as surfactants, builders, bleaches and polymers, see for example WO2004/074419. In order to make such a substitution commercially relevant, it should be done without a significant increase in cost and without loss of performance.

There is, therefore, a constant need for improving the detergency benefit provided by enzymes. One way to do this is to enhance the efficiency of the enzymes to their substrates. WO2004/053039 describes how an anti-redeposition endoglucanase is capable of enhancing the detergency performance of a protease, an amylase, a lipase, a hemicellulase and a pectinase.

### DETAILED DESCRIPTION OF THE INVENTION

One aspect of the present invention concerns the use of one or more glycoside hydrolase family 61 (GH61) polypeptide(s) to enhance the enzyme detergency benefit of one or more enzymes.

Another aspect of the present invention is a method for enhancing the enzyme detergency benefit of one or more enzymes in a cleaning or textile care process comprising the steps: a) combining one or more glycoside hydrolase family 61 (GH61) polypeptide(s) with said enzymes and b) performing the cleaning or textile care process, preferably in the presence of a detergent.

The detergency benefit enhancement of the present invention can be with respect to at least one of the following benefits: stain removal, anti-redeposition, whitening and/or textile care benefits such as softening, prevention or reduction of pilling, colour clarification, and/or dye transfer inhibition.

GH61 polypeptides have previously been applied in baking, where they have been shown to have an anti-staling effect, WO 04/031378. Furthermore, GH61 polypeptides have been applied in the conversion of cellulosic feedstock into ethanol, WO 05/074647, WO 05/074656, WO 07/089,290, and WO 09/033,071. These applications briefly mention that the polypeptides of the invention may be added to a detergent composition. There is, however, no indication in these applications that GH61 polypeptides are capable of enhancing the detergency effect of other enzymes in the detergent composition.

### DEFINITIONS

The term “anti-redeposition” as used herein describes the reduction or prevention of redeposition of soils dissolved or suspended in the wash liquor onto the cleaned objects. Redeposition may be seen after one or multiple washing cycles (e.g. as a greying, yellowing or other discolorations).

When used herein the term “coding sequence” means a polynucleotide, which directly specifies the amino acid sequence of its polypeptide product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

The term “enhancing effect” as used herein describes a detectable increased enzyme detergency benefit of one or more enzyme(s) during a washing and/or cleaning process where the enhancing effect is caused by the GH61 polypeptide, i.e. a boosting of the detergency benefit exerted by an enzyme due to the addition of a GH61 polypeptide. This enhancing effect can be perceived by spectroscopic methods such as measurement of the remission or by visual inspection (e.g. panel scores). The enhancing effect may be a direct effect, where the GH61 facilitates the enzyme action on a stain or it may act through an indirect effect, where compounds released by the action of enzymes on the stained textiles together with GH61 is enhancing the stain removal on other stains.

The term “enzyme detergency benefit” is defined herein as the advantageous effect an enzyme may add to a detergent compared to the same detergent without the enzyme. Important detergency benefits which can be provided by enzymes are stain removal resulting in no or very little visible soils after washing and or cleaning, prevention or reduction of redeposition of soils released in the washing process an effect that also is termed anti-redeposition, restoring fully or partly the whiteness of textiles, which originally were white but after repeated use and wash have obtained a greyish or yellowish appearance an effect that also is termed whitening. Textile care benefits, which are not directly related to catalytic stain removal or prevention of redeposition of soils are also important for enzyme detergency benefits. Examples of such textile care benefits are prevention or reduction of dye transfer from one fabric to another fabric or another part of the same fabric an effect that is also termed dye transfer inhibition or anti-backstaining, removal of protruding or broken fibers from a fabric surface to decrease pilling tendencies or remove already existing pills or fuzz an effect that also is termed anti-pilling, improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

The term “functional fragment of a polypeptide” is used to describe a polypeptide which is derived from a longer polypeptide, e.g., a mature polypeptide, and which has been truncated either in the N-terminal region or the C-terminal region or in both regions to generate a fragment of the parent polypeptide. To be a functional polypeptide the fragment must maintain at least 20%, preferably at least 40%, more preferably at least 50%, more preferably at least 60%, more preferably at least 70%, more preferably at least 80%, even more preferably at least 90%, most preferably at least 95%, and even most preferably at least 100% of the enzyme detergency enhancing effect of the full-length/mature polypeptide. GH61 polypeptides with a naturally occurring carbohydrate binding module (CBM) may be truncated such that CBM is removed to generate a functional CBM-free GH61 fragment, another GH61 functional fragment may constitute the GH61 domain (e.g. without signal peptide and linker sequences), functional fragments may also be polypeptides where less than 200 amino acids have been removed from the mature GH61 polypeptide, preferably less than 150 amino acids, more preferably less than 120, 100, 80, 60, 40, 30 amino acids, even more preferably less than 20 amino acids and most preferably less than 10 amino acids have been removed from the mature GH61 polypeptide.

The term “glycoside hydrolase family 61” or “GH61” is defined herein as a polypeptide falling into the glycoside hydrolase family 61 according to Henrissat B., 1991, Biochem. J. 280: 309-316, and Henrissat B., and Bairoch A., 1996, Biochem. J. 316: 695-696. Presently, Henrissat lists the GH61 Family as unclassified indicating that properties such as mechanism, catalytic nucleophile/base, catalytic proton donors, and 3-D structure are not known for the majority of polypeptides belonging to this family. In a preferred embodiment mature GH-61 polypeptides of the invention are defined as polypeptides comprising the following motifs:

[ILMV]−[QP]-x(4,5)-[AGS]-x−Y−[ILMV]-x-R-x-[EQ]-x(4)-[EHNQST] and
[EQ]-x-[YFW]-x(2)-[CG].
wherein the amino acids listed between square brackets “[ ]” indicate the acceptable amino acids for the given position for example: [ILMV] stands for Ile or Leu or Met or Val, x is any amino acid, x(2) is any amino acid at 2 contiguous positions, x(4,5) is any amino acid at 4 or 5 contiguous positions, and x(4) is any amino acid at 4 contiguous positions.

The parameter “identity” as used herein describes the relatedness between two amino acid sequences or between two nucleotide sequences. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

The term “isolated polynucleotide” as used herein refers to a polynucleotide that is isolated from a source. In one aspect, the isolated polynucleotide is at least at least 20% pure, more preferably at least 40% pure, more preferably at least 60% pure, even more preferably at least 80% pure, and most preferably at least 90% pure, and even most preferably at least 95% pure, as determined by agarose electrophoresis.

The term “isolated polypeptide” as used herein refers to a polypeptide that is isolated from a source. In one aspect, the variant or polypeptide is at least 20% pure, more preferably at least 40% pure, more preferably at least 60% pure, even more preferably at least 80% pure, most preferably at least 90% pure and even most preferably at least 95% pure, as determined by SDS-PAGE.

The term “host cell” as used herein includes any cell type that is susceptible to transformation, transfection, transduction, and the like with a nucleic acid construct or a vector comprising a polynucleotide of the present invention. The term “host cell” encompasses any progeny of a parent cell that is not identical to the parent cell due to mutations that occur during replication.

The term “operably linked” denotes herein a configuration in which a control sequence is placed at an appropriate position relative to the coding sequence of the polynucleotide sequence such that the control sequence directs the expression of the coding sequence of a polypeptide.

The term “substantially pure polynucleotide” as used herein refers to a polynucleotide preparation free of other extraneous or unwanted nucleotides and in a form suitable for use within genetically engineered polypeptide production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form. This can be accomplished, for example, by preparing the variant or polypeptide by well-known recombinant methods or by classical purification methods.

The term “stain removing enzyme” as used herein, describes an enzyme that aids the removal of a stain or soil from a fabric or a hard surface. Stain removing enzymes act on specific substrates, e.g. protease on protein, amylase on starch, lipase and cutinase on lipids (fats and oils), pectinase on pectin and hemicellulases on hemicellulose. Stains are often depositions of complex mixtures of different components which either results in a local discolouration of the material by itself or which leaves a sticky surface on the object which may attract soils dissolved in the washing liquor thereby resulting in discolouration of the stained area. When an enzyme acts on its specific substrate present in a stain the enzyme degrades or partially degrades its substrate thereby aiding the removal of soils and stain components associated with the substrate during the washing process. For example, when a protease acts on a grass stain it degrades the protein components in the grass and allows the green/brown colour to be released during washing.

The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

The term “whitening” as used herein describes the release of particulate soils re-deposited on the cleaned objects during washing. The re-deposited soils results in a general greying, yellowing or other discolorations of the object (textile, garment or hard surface). Whitening is preferably related to washing of light colored objects where it results in removal or diminishing of the grey or yellow appearance of the object. The term whitening may, however, also be used in relation to objects with darker coloration, where the whitening results in clarification or brightening of the colors.

### Use of GH61 to Enhance Enzyme Effect in Detergents

Enzymes are known to be “substrate specific”, i.e. each class of enzyme degrade a specific class of substances. For example, a protease can degrade proteins but cannot degrade starch. An amylase can degrade starch but cannot degrade proteins.

Because the soils and stains that are important for detergent formulators are composed of many different substances, a range of different enzymes, all with different substrate specificities have been developed for use in detergents both in relation to laundry and hard surface cleaning, such as dishwashing. These enzymes are considered to provide an enzyme detergency benefit, since they specifically improve stain removal in the cleaning process they are applied in as compared to the same process without enzymes. In a preferred embodiment of the present invention a GH61 polypeptide is used to enhance the enzyme detergency benefit (stain removal) of a stain removing enzyme. Specifically, addition of at least one GH61 polypeptide to a stain removing enzyme (one or more enzymes) increases the effect of the stain removing enzyme, as compared to when no GH61 is present, preferably in the presence of a detergent. Stain removing enzymes include enzymes such as protease, amylase, lipase, hemi-cellulase, in particular mannanase or xylanase, cutinase, and pectinase, in particular pectate lyase. Suitable and/or preferred enzymes are described in the “Detergency enzymes” section. Each of the enzymes described in this section can be selected individually or in combination and combined with a GH61 polypeptide to enhance the enzyme detergency benefit of the selected enzyme(s). In one embodiment a GH61 polypeptide is used to enhance the ability of a protease to remove of a protein containing stain. In another embodiment a GH61 polypeptide is used to enhance the ability of an amylase to remove of a starch containing stain. In another embodiment a GH61 polypeptide is used to enhance the ability of a lipase or cutinase to remove of a fat and/or oil containing stain. In another embodiment a GH61 polypeptide is used to enhance the ability of a mannanase to remove of a mannan and/or galactomanan containing stain. In another embodiment a GH61 polypeptide is used to enhance the ability of a pectinase to remove of a pectin containing stain.

The present inventors have tested eighteen GH61 polypeptides with very different sequences for their ability to enhance the enzyme detergency effect of either a single stain removing enzyme or an enzyme mixture. These GH61 polypeptides are described in further detail in the section “Glycoside hydrolase family 61 (GH61) polypeptides”. All the uses described in the present section (including those described below) encompass the general concept of using any GH61 polypeptide to enhance the enzyme detergency benefit of an enzyme and in particular one or more of the 18 GH61 polypeptides or functional fragments thereof disclosed in Table 1. It does also encompass the use of one or more of the specific GH61 polypeptides or polypeptide fragments described in the section “Glycoside hydrolase family 61 (GH61) polypeptides” as well as a polypeptide comprising an amino acid sequence which has at least 70% identity to one of these GH61 polypeptides, preferably at least 75%, 80%, 85%, 90%, 92%, 95%, 96%, 97%, 98% or 99% identity to one of these GH61 polypeptides or a functional fragment thereof.

All the GH61 polypeptides tested were capable of enhancing the stain removal effect of one or more enzymes selected from the group consisting of a protease, an amylase, a lipase and a mannanase. The GH61 enhancing effect on the enzymes was tested either on the individual enzymes or on an enzyme mixture. The GH61 enhancing effect was observed on at least one type of stain towards which the tested enzyme is expected to have an effect. The stain removing effect of an enzyme is dose-dependent, thus the stain removing effect increases with increasing enzyme dosages during the wash. However, at high concentrations of enzyme, the stain removing effect is not further increased by adding more of the enzyme, a plateau or a maximal enzyme benefit is reached. One effect that was observed was that a GH61 polypeptide was capable of boosting the stain removal at high enzyme concentration (maximal enzyme benefit had been reached) in that the GH61 polypeptide could lift the plateau of the stain removing effect. Another effect was seen at low enzyme concentrations (i.e. at concentrations below the plateau level, where the enzyme benefit can be increased by adding more enzyme) in that the addition of a GH61 polypeptide resulted in an increase in the stain removal effect without adding additional enzyme. In one embodiment of the present invention the enhanced stain removing effect is measured at an enzyme concentration below the plateau level, for example at an enzyme concentration corresponding to 20%, 30%, 40%, 50%, 60%, 70% or 80% of the enzyme concentration needed to reach the maximal enzyme benefit. Preferably it is measured at an enzyme concentration corresponding to 50% of the enzyme concentration needed to reach the maximal enzyme benefit. In another embodiment of the present invention the enhanced stain removing effect is measured at an enzyme concentration where the maximal enzyme benefit has been reached, i.e. at the plateau of the enzyme performance.

It is surprising that GH61 polypeptides are capable of enhancing the enzyme specificity of such a large variation of enzymes without having any specificity on its own towards the substrates of the stain removing enzymes. The present invention clearly illustrates that a wide range of different GH61 polypeptides can provide an enhanced enzyme detergency benefit on a wide range of soils and stains when used in combination with one or more enzymes, such as stain removing enzymes. Due to the missing specificity of the GH61 polypeptides, they are currently not believed to have an effect on their own. In an embodiment of the present invention the enhanced enzyme detergency benefit of the GH61 polypeptide together with a stain removing enzyme (one or more stain removing enzymes), is assessed as the enhanced removal of a stain selected form the group consisting of protein containing stains, starch containing stains, fat and/or oil containing stains and mannan containing stains. Examples of these types of stains are described under the individual enzymes in the “Detergency enzymes” section below.

The present inventors have, furthermore, found that at least five GH61 polypeptides also are capable of enhancing the enzyme detergency benefit of enzymes that prevent or reduce redepositing of soils on the washed objects (also termed anti-redeposition) or on enzymes which have a whitening effect. Redeposition of soils is believed to be due to the adherence of soil or pigments in the wash solution onto the surface of the cleaned objects. Soils which typically redeposit onto the cleaned objects are particulate soils such as carbon particles, clay, silica, peat moss, coffee grounds and metal oxides, such as ferric oxide, macromolecules such as fibers, hair, dust and polymers, colour molecules such as indigo. Furthermore, redeposition of colourless soils such as fats and oils, carbohydrates (e.g. starch and gums such as mannan, pectin and betaglucan) as well as proteins may indirectly result in greying of the object in that they produce sticky patches or films on the object which attract coloured soils. In particular starch has a tendency to generate sticky films on the washed objects even if only a small amount of starch is present in the wash solution. Fats and oils are hydrophobic and have a tendency to stick to hydrophobic objects such as plastic polymers and polyesters. Due to the complex internal structure and micro-porosity of cotton fibers fats and oils also have a tendency to accumulate within the capillary structure of the cotton where they are difficult to remove by washing and the fats and oils that are washed out redeposit onto clean polyester fabrics that are washed with the cotton fabrics. The fats and lipids may result in deposits of large amounts of divalent metal ions such as calcium and magnesium which over time may form an incrustation that may encompass various pigment soils which lead to a general gray appearance of the fabric.

Anti-redeposition and/or whitening enzymes are capable of reducing or preventing the redeposition of soils dissolved in the wash liquor onto the cleaned objects as well as releasing particulate soils or pigments bound on the washed objects. Proteases, lipases, amylases and hemicellulases function as anti-redeposition enzymes by preventing the redeposition of proteins, fats and oils, starch and gums onto the textile. Cellulases and xyloglucanases are also known as anti-redeposition and/or whitening enzymes. Cellulases and xyloglucanases contribute to whitening and anti-redeposition by selective actions that decrease the ability of cellulosic fibres to bind soil. The mechanistic interpretations are still incomplete, but it is clear that these effects are not related to degradation of the main semi-crystalline cellulose backbone of the cotton fibres. The cotton fibres also contain regions with very amorphous structure and it is reasonable to believe that these regions can influence the soil binding properties. By targeting these areas, release of bound soil and decreased binding of additional soil can be achieved. Example of anti-redeposition cellulases are cellulase complexes produced from fungal or bacterial sources such as Celluzyme® (Novozymes A/S), cellulase complexes with reduced CBH content, endo-beta-1,4-glucanases such as Endolase® and Celluclean® (Novozymes A/S) or xyloglucanases. The xyloglucasnases preferably have endo-glucanase side activity such as Whitezyme® (Novozymes A/S).

Redeposition is a serious concern, especially when the volume of water in the wash step is minimised in order to save energy or to limit consumption of clean water. Redeposition is also an issue when reducing levels of surfactants, polymers or builders during wash. In the near future, compaction of detergents and use of lower water volumes for washing will increase the importance of anti-redeposition tools. In an embodiment of the present invention a GH61 polypeptide is used to enhance the enzyme detergency benefit (anti-redepostion and/or whitening benefit) of an anti-redeposition enzyme and/or whitening enzyme, such as a lipase, protease, amylase, mannanase, cellulase and/or xyloglucanase. In a preferred embodiment the anti-redeposition enzyme and/or whitening enzyme is a cellulase or xyloglucanase, more preferably it is an endo-beta-1,4-glucanases or a xyloglucanase, preferably a xyloglucanase with endo-glucanase side activity. In a further embodiment the GH61 polypeptide is:
a) an isolated polypeptide comprising an amino acid sequence selected from the group consisting of:

| | |
|---|---|
| At1 | Amino acid residues 22 to 371 of SEQ ID NO: 3; |
| Tt5 | Amino acid residues 19 to 226 of SEQ ID NO: 8; |
| Hi1 | Amino acid residues 16 to 319 of SEQ ID NO: 5; |
| At2 | Amino acid residues 17 to 238 of SEQ ID NO: 13; and |
| Hi3 | Amino acid residues 20 to 296 of SEQ ID NO: 16; |

b) an isolated polypeptide comprising an amino acid sequence which has at least 70% identity to one of the amino acid sequences in a); or
c) a functional fragment of a) or b).

The cellulases and xyloglucanases are also known to provide additional fabric care benefits such as elimination or reduction of microfibrils and fuzz (anti-pilling), colour clarification, dye transfer inhibition, and/or softening of the fabric. These properties may also be enhanced by addition of one or more GH61 polypeptides.

In a preferred embodiment of the present invention one or more GH61 polypeptides are combined with at least two enzymes, more preferred at least three, four or five enzymes to enhance the stain removal effected by the specific enzymes, e.g. removal of stains containing protein, starch, fat, oil, mannan and/or pectin. Preferably, the enzymes have different substrate specificity, e.g. proteolytic activity, amylolytic activity, lipolytic activity, hemicellulytic activity or pectolytic activity. More preferably at least one of the enzymes is a protease (has proteolytic activity). The enzyme combination may for example be a protease with another stain removing enzyme, e.g. a protease and an amylase, a protease and a hemicellulase, a protease and a lipase, a protease and a cutinase, a protease and a pectinase or a protease with an anti-redeposition enzyme, preferably a protease and an endo-beta-1,4-glucanase or a protease and a xyloglucanase. More preferred the protease is combined with at least two stain removing enzymes. E.g. protease, lipase and amylase or protease, lipase and hemi-cellulase or protease, lipase and pectinase, or protease, lipase and cutinase, or protease, amylase and hemicellulase, or protease, amylase and pectinase, or protease, amylase and cutinase or protease, hemicellulase and pectinase, or protease, hemicellulase and cutinase or protease, pectinase and cutinase. Even more preferred the protease is combined with at least three stain removing enzymes, e.g. protease, amylase, lipase and hemicellulase or protease, amylase, lipase and pectinase or protease, amylase, hemicellulase and pectinase or protease, lipase, hemicellulase and pectinase.

The enhancement or improvement in the detergency benefit in relation to stain removal, prevention or reduction of redeposition of soils and/or whitening can be assessed by measuring the light remission of the object before treatment and after treatment for example as described in the Method and Materials section in the Examples of this application. Stain removal, anti-redeposition and whitening may also be measured by alternative methods such as visual inspection using for example panel score evaluations, FTIR spectroscopy (Fourrier Transformed Infra-Red spectroscopy), microscopy, various extraction procedures, or colorimetric assays. In a preferred embodiment of the present invention the enzyme detergency benefit of the enzyme(s) is enhanced by at least 1 delta remission unit, preferably at least 1.25, more preferably at least 1.5, more preferably at least 1.75, most preferably at least 2, even more preferably at least 2.25 or 2.5 and even most preferably at least 2.75 or 3 delta remission units when the enzyme(s) is combined with a glycosyl hydrolase family 61 polypeptide as compared to when the enzyme(s) is used without the glycosyl hydrolase family 61 polypeptide when the assessment is performed as described in the Materials and Method section using the Laundrometer set-up A at a water hardness of 24° FH for stain removal benefits or using the Small scale anti-redeposition washing method for anti-redeposition benefits. In an alternative embodiment the Laundrometer set-up B at a water hardness of 24° FH is used to assess stain removal benefits achieving similar enhancement in delta remission units as for set-up A.

The enzyme detergency benefit concept is not restricted to the benefit which can be observed in relation to stain removal or anti-redeposition or whitening as described above. In relation to laundry the enzyme detergency benefit concept also encompasses textile care benefits such as softening, removal or reduction of fuzz and pills, colour clarification, or inhibition of dye transfer. These enzyme detergency benefits are often provided by the interaction of cellulases, cellulases with reduced CBH content, endoglucanases or xyloglucanases, preferably with endoglucanase side activity on the cellulolytic fibers in the textiles. However, it is also known that lipases and/or cutinases may prevent or reduce pill formation in textile products comprising polyester (see for example WO 01/34899). It is, therefore, expected that when GH61 polypeptides can enhance the enzyme detergency benefit in relation to anti-redeposition and whitening on textiles they will also be able to enhance the enzyme detergency benefit in relation to other textile care benefits provided by enzymes with cellulolytic activity or lipolytic activity. The removal or reduction of fuzz and pills and colour clarification can be assessed as described on page 139 to 141 in Enzymes in Detergency, 1997, edited by van Ee, Misset and Bass, published by Dekker. Softening effects may be assessed as described on page 144 to 145 in Enzymes in Detergency, 1997, edited by van Ee, Misset and Bass, published by Dekker. Reduction or inhibition of dye transfer may be measured as described in Example 9 of U.S. Pat. No. 5,700,770 (hereby incorporated by reference). This method is not limited to peroxidases and oxidases it can also be extended to cellulases and xyloglucanase with dye transfer inhibition properties.

In a further embodiment of the present invention a glycosyl hydrolase family 61 polypeptide and the enzyme(s) are applied in a cleaning process or a textile care process. The cleaning process or the textile care process may for example be a laundry process, other cleaning processes are a dishwashing process, or cleaning of hard surfaces such as bathroom tiles, floors, table tops, drains, sinks and washbasins. Laundry processes can for example be household laundering, but it may also be industrial laundering. Furthermore, the invention relates to a process for laundering of textiles, fabrics and/or garments where the process comprises treating fabrics with a washing solution containing a detergent composition, and at least one enzyme and a GH61 polypeptide or a GH61 polypeptide composition and stained textile, where the stains could belong to the group: natural food based stains, technical stains, protein containing stains such as dairy products, egg, grass, body soils, blood, mud and baby food, fat containing stains such as lipstick, body soils, mayonnaise, mustard, salad dressing, butter and gravey, carbohydrate containing stains such as rice, potatoes, cereals, noodles, pasta and porridge, particulate stains such as clay, mud and soil, colored stains such as blood, ink, grass and chocolate, stains from plant materials including grass, spices like paprika, tomatoes, cocoa, guar gum and locust bean gum, tea, wine and coffee, stains from humans including sweat, sebum, blood and feeces, and other stains such as mineral oils such as dirty motor oil, mechanical grease. In particular textiles containing stains selected from the group consisting of dirty motor oil, starch, milk or dairy products, grass, oil, blood, cocoa, tea and particulate soil or clay stains. The cleaning process or a textile care process can for example be carried out in a machine washing process or in a manual washing process. The washing solution can for example be an aqueous washing solution containing a detergent composition. The aqueous washing solution can have a pH from 3 to 12, preferably pH 6 to 11 or pH7.5 to 10.5, more preferably pH 8 to 10 or 9.5 to 11. The water hardness of the aqueous washing solution is preferably below 100° FH, preferably below 90° FH, more preferably below 80° FH, 70° FH, 60° FH or 50° FH, even more preferably below 48° FH, most preferably below 24° FH. The concentration of the detergent composition in the washing solution can be in the range of 0.1 to 10 g detergent composition /l of wash solution, preferably from 0.25 to 9 g/l, 0.5 to 8 g/l, 0.75 to 7 g/l, or 1 to 6 g detergent composition /l of wash solution, more preferably from 1.25 to 5.5 g/l or 1.5 to 5 g detergent composition /l of wash solution, more preferably from 2 to 4 g detergent composition /l of wash solution, most preferably from 2.5 to 3 g detergent composition /l of wash solution.

The textiles, fabrics and/or garments subjected to a washing, cleaning or textile care process of the present invention may be conventional washable laundry, for example household laundry. Preferably, the major part of the laundry is garments and fabrics, including knits, wovens, denims, non-wovens, felts, yarns, and towelling. The fabrics may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The fabrics may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). In a preferred embodiment the textiles subjected to washing comprise cellulose based textiles, preferably cotton. In another preferred embodiment the textiles subjected to washing comprise synthetic polymer textiles, preferably polyester.

The last few years compaction of detergents has been an increasing focus area of detergent manufactures. By reducing the volume and weight of various powder and liquid detergents without reducing the number of washes and wash performance, huge environmental benefits may be achieved, in terms of reducing the amount of detergents, packaging and transport used. Similarly, there is an interest in replacing components in detergents, which is derived from petrochemicals with biological components which are from a renewable source such as enzymes and polypeptides.

A further aspect of the present invention is the use of one or more GH61 polypeptide(s) for compaction of the detergent in which it is used. Compaction may for example be achieved by substituting some of the major detergent ingredients such as builders, fillers or surfactants with GH61 polypeptide in combination with one or more enzymes, such as the stain removing or anti-redeposition or textile care enzymes described herein without affecting the overall wash performance of the detergent. Compaction may also be the reduction of the amount of gram detergent formulation per liter of wash solution without affecting the overall wash performance that can be achieved with a full dose detergent. For example if a particular enzyme containing detergent formulation is used at 5 g/L of wash solution, then the addition of one or more GH61 polypeptide(s) can reduce the amount of detergent pr. liter of wash solution to the range of 2.5 g/L to 4.75 g/l corresponding to a 5% to 50% reduction without affecting the wash performance of the detergent. In a preferred embodiment the amount of detergent pr. liter of wash solution is reduced from 5 g/L to the range of 0.5 g/L to 4.5 g/L or preferably from 1 g/L to 4.0 g/L or most preferably from 1.5 g/L to 3.5 g/L, corresponding to a 10% to 90% reduction or a 20% to 80% reduction or a 30% to 70% reduction, respectively, without affecting the wash performance of the detergent. In a preferred embodiment of the present invention a GH61 polypeptide is used in combination with one or more enzymes to reduce the dose of a liquid or powder detergent to half the recommended normal dose of a corresponding detergent without GH61 polypeptide in a washing process where the reduced dose provide the same cleaning benefit as when dosed in the recommended normal dose. Another preferred embodiment is a method of washing and/or cleaning, wherein a detergent comprising a GH61 polypeptide is dosed in an amount that is at least 5%, preferably at least 10%, more preferably at least 20%, 30%, 40%, most preferably at least 50% and even most preferably at least 75% by weight lower than a corresponding detergent without GH61 polypeptides and where at least the same detergent benefit is obtained with the reduced dose of the GH61 polypeptide-containing detergent when compared to the corresponding detergent without GH61 polypeptides. The corresponding detergent is a detergent with exactly the same composition as the GH61 polypeptide-containing detergent, except that the corresponding detergent does not contain any GH61 polypeptides with an enzyme detergency enhancing effect, preferably the corresponding detergent is completely free of GH61 polypeptides. Another preferred embodiment is a method of washing and/or cleaning, wherein a detergent comprising a GH61 polypeptide is dosed in the range of 0.1 to 5.5 g detergent/l of wash solution, preferably in the range of 0.5 to 5 g/l, 1 to 4.5 g/l or 1.5 to 4.0 g detergent/L of wash solution, more preferably in the range of 2 g detergent/L of wash solution to 3.5 g detergent/L of wash solution, most preferably in the range of 2.5 g detergent/L of wash solution to 3 g detergent/L of wash solution.

### Detergency Enzymes

GH61 polypeptides can potentially be used to enhance the enzyme detergency effect of any enzyme considered useful in detergents. Such an enzyme is preferably suitable for use in the pH range from pH 6 to 12, preferably from 7 to 11 more preferably from 7.5 to 10, most preferably from pH 8 to 9.5. Detergency enhancing enzyme to be used with the present invention may for example be selected among one or more of the following enzymes: proteases, cellulases, hemicellulases, lipases, cutinases, amylases and pectinases. Preferred enzymes are selected from the group consisting of proteases, amylases, lipases, mannanases and endoglucanases. More preferred enzymes are selected from the group consisting of metalloprotease, serine protease, triacylglycerol lipase, phospholipase A2, phospholipase A1, endoglucanses, xyloglucanases, alpha-amylases, laccases, pectate lyases, xylanases, and mannanases. Even more preferred is stain removing enzymes selected from the group consisting of proteases, alpha-amylases, lipases, and mannanases. Below are more detailed descriptions of individual enzymes which may be used together with a GH61 polypeptide.

### Proteases

In a preferred embodiment a GH61 is used together with a protease or a proteolytic enzyme to provide improved detergency performance on soils that contain protein. Common protein stains may for example comprise blood, dairy products, body soils (sebum), baby formula, mud, grass, eggs and baby food without excluding other protein containing substances.

Any protease suitable for use in alkaline solutions can be used. Suitable proteases include those of animal, vegetable or microbial origin. Microbial origin is preferred. Chemically modified or protein engineered mutants are included. The protease may for example be a metalloprotease (EC 3.4.17 or EC 3.4.24) or a serine protease (EC 3.4.21), preferably an alkaline microbial protease or a trypsin-like protease. Examples of alkaline proteases are subtilisins (EC 3.4.21.62), especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Preferred commercially available protease enzymes include Alcalase®, Savinase®, Liquanase®, Coronase®, Polarzyme®, Everlase®, Esperase®, and Kannase® (Novozymes A/S), Maxatase®, Maxacal®, Maxapem®, Properase®, Purafect Prime®, Purafect®, Purafect OxP®, FN2™, and FN3™ (Genencor International Inc.).

Protease enzymes may be incorporated into detergent compositions in accordance with the invention at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition

### Lipases

In another preferred embodiment the GH61 is used together with a lipase or a lipolytic enzyme to provide improved detergency performance on soils that contain fat or oil. Common fat and/or oil containing stains may for example comprise body soils (sebum), lipstick, mayonnaise, mustard, salad dressings, vegetable fat and oil, animal fat (e.g. butter and gravy), wax and mineral oil without excluding other oil and/or fat containing substances.

Any lipase suitable for use in alkaline solutions can be used. Suitable lipases include those of bacterial or fungal origin. Chemically or genetically modified mutants of such lipases are included in this connection. The lipase may for example be triacylglycerol lipase (EC3.1.1.3), phospholipase A2 (EC 3.1.1.4), Lysophospholipase (EC 3.1.1.5), Monoglyceride lipase (EC 3.1.1.23), galactolipase (EC 3.1.1.26), phospholipase A1 (EC 3.1.1.32), Lipoprotein lipase (EC 3.1.1.34). Examples of useful lipases include a Humicola lanuginosa lipase, e.g. as described in EP 258 068 and EP 305 216; a Rhizomucor miehei lipase, e.g. as described in EP 238 023 or from H. insolens as described in WO 96/13580; a Candida lipase, such as a C. antarctica lipase, e.g. the C. antarctica lipase A or B described in EP 214 761; a Pseudomonas lipase, such as one of those described in EP 721 981 (e.g. a lipase obtainable from a Pseudomonas sp. SD705 strain having deposit accession number FERM BP-4772), in PCT/JP96/00426, in PCT/JP96/00454 (e.g. a P. solanacearum lipase), in EP 571 982 or in WO 95/14783 (e.g. a P. mendocina lipase), a P. alcaligenes or P. pseudoalcaligenes lipase, e.g. as described in EP 218 272, a P. cepacia lipase, e.g. as described in EP 331 376, a P. stutzeri lipase, e.g. as disclosed in GB 1,372,034, or a P. fluorescens lipase; a Bacillus lipase, e.g. a B. subtilis lipase (Dartois et al. (1993) Biochemica et Biophysica Acta 1131:253-260), a B. stearothermophilus lipase (JP 64/744992) and a B. pumilus lipase (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202. A preferred lipase variant is that of Humicola lanuginosa DSM 4109 as described in WO 00/60063. Especially preferred are the variants disclosed in the Example in WO 00/60063 with improved first wash performance., i.e., T231R+N233R;G91A+D96W+E99K+G263Q+L264A+1265T+G266D+T267A+L269N+R209P+T231R+N233R;N33Q+D96S+T231R+N233R+Q249R;E99N+N101S+T231R+N233R+Q249R; E99N+N101S+T231R+N233R+Q249R.

Suitable commercially available lipases include Lipex®, Lipolase® and Lipolase Ultra®, Lipolex®, Lipoclean® (available from Novozymes A/S), M1 Lipase™ and Lipomax™ (available from Genencor Inc.) and Lipase P “Amano” (available from Amano Pharmaceutical Co. Ltd.). Commercially available cutinases include Lumafast™ from Genencor Inc.

The lipases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Cutinases

In another preferred embodiment the GH61 is used together with a lipolytic enzyme to provide improved detergency performance on soils that contain fat or oil. Potentially useful types of lipolytic enzymes include cutinases (EC 3.1.1.74), e.g. a cutinase derived from Pseudomonas mendocina as described in WO 88/09367, or a cutinase derived from Fusarium solani pisi (described, e.g., in WO 90/09446). Due to the lipolytic activity of cutinases they may be effective against the same stains as lipases. Commercially available cutinases include Lumafast™ from Genencor Inc.

The cutinases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Carbohydrases

Carbohydrases covers glycoside hydrolases (EC 3.2.1.−) and polysaccharide lyases (EC 4.2.2.−). Glycoside hydrolases catalyze the hydrolysis of the glycosidic bond between two or more carbohydrates or between a carbohydrate and a non-carbohydrate moiety. Polysaccharide lyases catalyze the cleavage of polysaccharide chains by a beta elimination mechanism resulting in a double bond of the newly formed reducing end. Carbohydrases include for example amylases, hemicellulases, pectinases and cellulases described in more detail below. Other carbohydrases may be xanthanases or pullulanases.

Xanthanases can be used to degrade xanthan gum which is used as thickener in the food industry. Suitable xanthanases include those of bacterial or fungal origin. Chemically or genetically modified mutants are included. Sources of xanthanases are for example described in Cadmus et al, 1988, J of Industrial Microbiology and Biotechnology: 4: 127-133; EP0030393 and Hashimoto et al, 1998, Appl Environ Microbiol.: 64: 3765-3768.

Pullulanase is a debranching enzyme which may aid the access of other carbohydrases to a substrate and thereby aid the degradation of the substrate. Suitable pullulanase include those of bacterial or fungal origin. Chemically or genetically modified mutants are included. Sources of pullulanase are for example Dextrozyme® and Promozyme® D2 (Novozymes A/S).

### Amylases

In a preferred embodiment a GH61 is used together with an amylase or an amylolytic enzyme to provide improved detergency performance on soils that contain starch. Common starch containing stains may for example comprise rice, potato, cereals, noodles, pasta and porridge, without excluding other starch containing substances. Starch stains may not always be visible to the naked eye but starch stains tend to act as glue for particulate soils in wash solutions. Amylases prevent the buildup of starch deposits which may cause discoloration on fabrics and starch films on dishes.

Amylases comprise e.g. alpha-amylases (EC 3.2.1.1), beta-amylases (EC 3.2.1.2) and/or glucoamylases (EC 3.2.1.3) of bacterial or fungal origin. Chemically or genetically modified mutants of such amylases are included in this connection. Alpha-amylases are preferred in relation to the present invention. Relevant alpha-amylases include, for example, α-amylases obtainable from Bacillus species, in particular a special strain of B. licheniformis, described in more detail in GB 1296839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Further examples of useful amylases are the alpha-amylases derived from Bacillus sp. strains NCIB 12289, NCIB 12512, NCIB 12513 and DSM 9375; the alpha-amylases shown in SEQ ID NO 1 and 2 of WO 95/26397 (hereby incorporated by reference); the AA560 alpha-amylase derived from Bacillus sp. DSM 12649 disclosed as SEQ ID NO: 2 in WO 00/60060 (hereby incorporated by reference) and the variants of the AA560 alpha-amylase, including the AA560 variant disclosed in Example 7 and 8 (hereby incorporated by reference).

Relevant commercially available amylases include Natalase®, Stainzyme®, Duramyl®, Termamyl®, Termamyl™ Ultra, Fungamyl® and BAN® (all available from Novozymes A/S, Bagsvaerd, Denmark), and Rapidase® and Maxamyl® P (available from DSM, Holland) and Purastar®, Purastar OxAm and Powerase™ (available from Danisco A/S).

Other useful amylases are CGTases (cyclodextrin glucanotransferases, EC 2.4.1.19), e.g. those obtainable from species of Bacillus, Thermoanaerobactor or Thermoanaerobacterium.

The amylases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Hemicellulases

In another preferred embodiment a GH61 is used together with a hemi-cellulase to provide improved detergency performance on soils that contain hemi-cellulose. Hemicelluloses are the most complex group of non-starch polysaccharides in the plant cell wall. They consist of polymers of xylose, arabinose, galactose, mannose and/or glucose which are often highly branched and connected to other cell wall structures. Hemicellulases of the present invention therefore include enzymes with xylanolytic activity, arabinolytic activity, galactolytic activity and/or mannolytic activity. The hemi-cellulases of the present invention may for example be selected from xylanases (EC 3.2.1.8, EC 3.2.1.32, and EC 3.2.1.136), xyloglucanases (EC 3.2.1.4 and EC 3.2.1.151), arabinofuranosidases (EC 3.2.1.55), acetylxylan esterases (EC EC 3.1.1.72), glucuronidases (EC 3.2.1.31, EC 3.2.1.56, 3.2.1.128 and 3.2.1.139), glucanohydrolase (EC 3.2.1.11, EC 3.2.1.83 and EC 3.2.1.73), ferulic acid esterases (EC 3.1.1.73), coumaric acid esterases (EC 3.1.1.73), mannanases (EC 3.2.1.25; EC 3.2.1.78 and EC 3.2.1.101), arabinosidase (EC 3.2.1.88), arabinanases (EC 3.2.1.99), galactanases (EC 3.2.1.89, EC 3.2.1.23 and 3.2.1.164) and lichenases (EC 3.2.1.73). This is, however, not to be considered as an exhausting list.

Mannananase is a preferred hemicellulase in relation to the present invention. Mannanases hydrolyse the biopolymers made up of galactomannans. Mannan containing stains often comprise guar gum and locust bean gum, which are widely used as stabilizers in food and cosmetic products. Suitable mannanases include those of bacterial or fungal origin. Chemically or genetically modified mutants are included. In a preferred embodiment the mannanase is derived from a strain of the genus Bacillus, especially Bacillus sp. 1633 disclosed in positions 31-330 of SEQ ID NO:2 or in SEQ ID NO: 5 of WO 99/64619 (hereby incorporated by reference) or Bacillus agaradhaerens, for example from the type strain DSM 8721. A suitable commercially available mannanase is Mannaway® produced by Novozymes A/S or Purabrite™ produced by Genencor a Danisco division.

Xylanase is a preferred hemicellulase in relation to the present invention. A suitable commercially available xylanase is Pulpzyme® HC (available from Novozymes A/S).

The hemicellulases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Pectinases

In another preferred embodiment a GH61 is used together with a pectinase or pectolytic enzyme to provide improved detergency performance on pectinaceous soils. The term pectinase or pectolytic enzyme is intended to include any pectinase enzyme defined according to the art where pectinases are a group of enzymes that catalyze the cleavage of glycosidic linkages. Basically three types of pectolytic enzymes exist: pectinesterase, which only removes methoxyl residues from pectin, a range of depolymerizing enzymes, and protopectinase, which solubilizes protopectin to form pectin (Sakai et al., (1993) Advances in Applied Microbiology vol 39 pp 213-294). Example of a pectinases or pectolytic enzyme useful in the invention is pectate lyase (EC 4.2.2.2 and EC 4.2.2.9), polygalacturonase (EC 3.2.1.15 and EC 3.2.1.67), polymethyl galacturonase, pectin lyase (EC 4.2.2.10), galactanases (EC 3.2.1.89), arabinanases (EC 3.2.1.99) and/or pectin esterases (EC 3.1.1.11). Pectinaceous soils or stains may for example be composed of pectate, polygalacturonicacid, and/or pectin which may be esterified to a higher or lower degree. These substrates are common in soils of vegetable origin which may include grass, vegetables such as spinach, beetroot, carrot, tomatoes, fruits such as all types of cherries and berries, peach, apricot, mango, bananas and grapes as well as stains from drinks derived from plant material, such as wine, beer, fruit juices and additionally tomato sauce, jellies or jams without excluding other pectin containing substances.

Suitable pectinolytic enzymes include those described in WO 99/27083, WO 99/27084, WO 00/55309 and WO 02/092741.

Suitable pectate lyases include those of bacterial or fungal origin. Chemically or genetically modified mutants are included. In a preferred embodiment the pectate lyase is derived from a strain of the genus Bacillus, especially a strain of Bacillus substilis, especially Bacillus subtilis DSM14218 disclosed in SEQ ID NO:2 or a variant thereof disclosed in Example 6 of WO 02/092741 (hereby incorporated by reference) or a variant disclosed in WO 03/095638 (hereby incorporated by reference). Alternatively the pectate lyase is derived from a strain of Bacillus licheniformis, especially the pectate lyases disclosed as SEQ ID NO: 8 in WO 99/27083 (hereby incorporated by reference) or variants thereof as described in WO 02/06442.

Suitable commercially available pectate lyases are XPect®, Pectaway® or Pectawash® produced by Novozymes A/S.

The pectinolytic enzymes are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Cellulases

In another preferred embodiment a GH61 is used together with a cellulase to provide improved detergency performance. Cellulases are primarily used for textile care, such as removal or reduction of fuzz and pills from cotton fabrics, softening, colour clarification, particulate soil removal, dye transfer inhibition and anti-redeposition of soils on cotton fabrics in the wash.

Any cellulase suitable for use in alkaline solutions can be used. Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases (EC 3.2.1.4). Some xyloglucanases may also have endoglucanases activity and are also considered as suitable cellulases in the present invention. Suitable cellulases are disclosed in U.S. Pat. No. 4,435,307, which discloses fungal cellulases produced from Humicola insolens. Especially suitable cellulases are the cellulases having textile care benefits. Examples of such cellulases are cellulases described in European patent application No. 0 495 257.

Suitable mono-component endoglucanases may be obtained from one or more of the following species Exidia glandulosa, Crinipellis scabella, Fomes fomentarius, Spongipellis sp., Rhizophlyctis rosea, Rhizomucor pusillus, Phycomyces nitens, and Chaetostylum fresenii, Diplodia gossypina, Microsphaeropsis sp., Ulospora bilgramii, Aureobasidium sp., Macrophomina phaseolina, Ascobolus stictoides, Saccobolus dilutellus, Peziza, Penicillium verruculosum, Penicillium chrysogenum, and Thermomyces verrucosus, Trichoderma reesei aka Hypocrea jecorina, Diaporthe syngenesia, Colletotrichum lagenarium, Xylaria hypoxylon, Nigrospora sp., Nodulisporum sp., and Poronia punctata, Cylindrocarpon sp., Nectria pinea, Volutefla colletotrichoides, Sordaria fimicola, Sordaria macrospora, Thielavia thermophila, Syspastospora boninensis, Cladorrhinum foecundissimum, Chaetomium murorum, Chaetomium virescens, Chaetomium brasiliensis, Chaetomium cunicolorum, Myceliophthora thermophila, Gliocladium catenulatum, Scytalidium thermophila, Acremonium sp Fusarium solani, Fusarium anguioides, Fusarium poae, Fusarium oxysporum ssp. lycopersici, Fusarium oxysporum ssp. passiflora, Humicola nigrescens, Humicola grisea, Fusarium oxysporum, Thielavia terrestris or Humicola insolens. One preferred endoglucanase is disclosed in WO 96/29397 as SEQ ID NO: 9 (hereby incorporated by reference) or an enzyme with at least 70% identity thereto and variants thereof as disclosed in Example 1 of WO 98/12307. Another preferred endoglucanase is disclosed in WO 91/017243 (SEQ ID NO:2) or endoglucanases variants as disclosed in WO 94/007998.

Endoglucanases with an anti-redeposition effect may be obtained from fungal endoglucanases lacking a carbohydrate-binding module (CBM) from a number of bacterial sources. Some sources are Humicola insolens, Bacillus sp. deposited as DSM 12648, Bacillus sp. KSMS237 deposited as FERM P-16067, Panibacillus polymyxa, and Panibacillus pabuli. Specific anti-redeposition endoglucanase are disclosed in WO 91/17244 (FIG. 14) (hereby incorporated by reference), WO 04/053039 SEQ ID NO: 2 (hereby incorporated by reference), JP 2000210081 position 1 to 824 of SEQ ID NO: 1 (hereby incorporated by reference).

Xyloglucanases with an anti-redeposition effect may be obtained from a number of bacterial sources. Some sources are Bacillus licheniformis, Bacillus agaradhaerens, (WO 99/02663) Panibacillus polymyxa, and Panibacillus pabuli (WO01/62903). Suitable variants of xyloglucasnes are also described in PCT/EP2009/056875. A commercially available xyloglucanase is Whitezyme® (Novozymes A/S).

Commercially available cellulases include Celluclast® produced from Trichoderma reesei, Celluzyme® produced from Humicola insolens. Commercially available endoglucanases are Carezyme®, Renozyme®, Endolase® and Celluclean® (Novozymes A/S), and KAC-500(B)™ (Kao Corporation) and Clazinase™, Puradax™ EG L and Puradax HA (Danisco A/S).

Cellulases are normally incorporated in the detergent composition at a level of from 0.000001% to 2% of enzyme protein by weight of the composition, preferably at a level of from 0.00001% to 1% of enzyme protein by weight of the composition, more preferably at a level of from 0.0001% to 0.5% of enzyme protein by weight of the composition, even more preferably at a level of from 0.001% to 0.2% of enzyme protein by weight of the composition.

### Peroxidases/Oxidases

In another preferred embodiment a GH61 is used together with a peroxidase or oxidase to provide improved detergency performance. Peroxidases and oxidases may be used in relation to bleaching of localized stains on fabrics, tableware and other hard surfaces, disinfection and odour removal/prevention and removal of hydrogen peroxide after bleaching.

Suitable peroxidases/oxidases include those of plant, bacterial or fungal origin. Chemically modified or protein engineered mutants are included. Examples of useful oxidases are laccases (EC 1.10.3.2). Examples of useful peroxidases include catalases (EC 1.11.1.6) and peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

### Arylesterase

In another preferred embodiment a GH61 is used together with an arylesterase to provide improved detergency performance. Arylesterase (EC 3.1.1.2) also termed perhydrolase, Aesterase, paraoxonase, or aromatic esterase may be used in relation to bleaching, in particular textile bleaching as described in WO 2007/136469.

Suitable arylesterases include those of plant, bacterial or fungal origin. Chemically modified or protein engineered mutants are included. Examples of useful arylesterase are for example obtained from M. Smegmatis as described in WO 2005/056782. Other enzymes with perhydrolase activity:

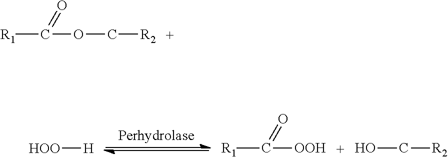

which are not necessarily classified as arylesterases are also included in this term for the purpose of the present invention.

### Glycoside Hydrolase Family 61 (GH61) Polypeptides

The present invention relates to the use of isolated GH61 polypeptides in general. Preferably, the GH61 polypeptide used in the present invention has a pyrogallol activity of at least 0.15 absorbance units when measured in the pyrogallol activity assay in Example 33 using 0.1 mg/mL GH61 polypeptide, more preferably the GH61 polypeptide has a pyrogallol activity of at least 0.2 absorbance units, even more preferably of at least 0.25 absorbance units. It is also preferred that the GH61 polypeptide used in the present invention capable of enhancing the enzyme detergency benefit of an enzyme by at least 1 delta remission units compared to when the enzyme is used without the GH61 polypeptide, more preferred it is capable of enhancing the detergency benefit by 2 delta remission units, more preferably by 3 delta remission units and most preferably by 5 delta remission units.

The invention has been demonstrated using eighteen different isolated GH61 polypeptides with very different sequence identities. Some of these are already publicly available, either as fully expressed proteins or in sequence databases as open reading frames resulting from genome sequencing projects (indicated with reference in Table 1).

Table 1 is a list of the GH61 polypeptides which has been used to illustrate the present invention and which can be applied in all the uses of the present invention.

TABLE 1

| SEQ | | | | | |
|---|---|---|---|---|---|
| ID | Mature | GH61 | | Short | |
| NO | protein | domain | Organism | name | Reference |
| 1 | 18-233 | 18-233 | Thielavia terrestris | Tt1 | WO2004/031378 |
| 2 | 20-304 | 20-225 | Thielavia terrestris | Tt2 | WO2005/074647 |
| 3 | 22-371 | 22-252 | Aspergillus terreus | At1 | UnitProt |
| | | | | | Q0CEG4 |
| 4 | 21-330 | 21-241 | Neurospora crassa | Nc1 | UniProt |
| | | | | | Q7RWN7 |
| 5 | 16-319 | 16-243 | Humicola insolens | Hi1 | WO2004/031378 |
| 6 | 20-326 | 20-243 | Thielavia terrestris | Tt3 | WO2005/074647 |
| 7 | 18-239 | 18-239 | Thielavia terrestris | Tt4 | WO2005/074647 |
| 8 | 19-226 | 19-226 | Thielavia terrestris | Tt5 | WO2005/074647 |
| 9 | 21-225 | 21-225 | Poronia punctata | Pp1 | |
| 10 | 20-298 | 20-236 | Humicola insolens | Hi2 | |
| 11 | 18-246 | 18-246 | Verticillium tenerum | Vt1 | |
| 12 | 17-234 | 17-234 | Verticillium tenerum | Vt2 | |
| 13 | 17-238 | 17-238 | Aspergillus terreus | At2 | UniProt Q0CDX1 |
| 14 | 21-259 | 21-259 | Chaetomium | Cg1 | |
| | | | globosum | | |
| 15 | 22-249 | 22-249 | Thermoascus | Ta1 | WO2005/074656 |
| | | | aurantiaticus | | |
| 16 | 20-296 | 20-224 | Humicola insolens | Hi3 | |
| 17 | 20-248 | 20-248 | Aspergillus terreus | At3 | |
| 18 | 21-298 | 21-245 | Aspergillus terreus | At4 | UniProt Q0C7Z0 |

The sequence of the mature protein is indicated as the amino acid residues of the respective SEQ ID NO without the predicted signal peptide. A mature GH61 polypeptide generally starts with a Histidine at the N-terminal. The histidine may either be in a methylated form or in an unmethylated form. A composition of GH61 polypeptides may comprise 95-100% unmethylated GH61 or 95-100% methylated GH61. The GH61 composition may also be composed of a combination of methylated and unmythylated GH61, for example about 25% can be unmethylated and about 75% methylated, or about 40% can be unmethylated and 60% methylated or about 50% methylated and unmethylated or about 60% unmethylated and 40% methylated or about 75% unmethylated and about 25% methylated. In addition to the signal peptide, the GH61 polypeptides may comprise linkers, carbohydrate binding modules (CBM) and other non-specific areas. When the amino acids constituting these areas are removed the GH61 domain remains. The amino acid residues corresponding to the GH61 domains of the GH61 polypeptides used to illustrate the present invention is given in table 1.

The sequence identity between the 18 GH61 polypeptides in table 1 is given below. The identities corresponds to the number of exact matches divided by the total length of the alignment excluding the gaps and are calculated as indicated in the definitions.

| | ID1 | ID2 | ID3 | ID4 | ID5 | ID6 | ID7 | ID8 | ID9 |
|---|---|---|---|---|---|---|---|---|---|
| ID1 | 100.00 | 46.15 | 36.32 | 48.33 | 44.98 | 52.56 | 37.67 | 53.78 | 43.38 |
| ID2 | 46.15 | 100.00 | 36.45 | 47.57 | 39.79 | 47.10 | 39.52 | 42.18 | 65.18 |
| ID3 | 36.32 | 36.45 | 100.00 | 40.27 | 37.50 | 40.20 | 40.45 | 38.65 | 31.31 |
| ID4 | 48.33 | 47.57 | 40.27 | 100.00 | 42.42 | 72.09 | 40.85 | 49.51 | 39.82 |
| ID5 | 44.98 | 39.79 | 37.50 | 42.42 | 100.00 | 44.03 | 40.57 | 39.07 | 34.11 |
| ID6 | 52.56 | 47.10 | 40.20 | 72.09 | 44.03 | 100.00 | 39.17 | 51.21 | 44.39 |
| ID7 | 37.67 | 39.52 | 40.45 | 40.85 | 40.57 | 39.17 | 100.00 | 37.32 | 38.57 |
| ID8 | 53.78 | 42.18 | 38.65 | 49.51 | 39.07 | 51.21 | 37.32 | 100.00 | 38.99 |
| ID9 | 43.38 | 65.18 | 31.31 | 39.82 | 34.11 | 44.39 | 38.57 | 38.99 | 100.00 |
| ID10 | 33.33 | 43.91 | 33.46 | 39.41 | 43.38 | 38.85 | 30.91 | 35.75 | 39.34 |
| ID11 | 45.24 | 42.72 | 41.63 | 43.05 | 56.61 | 43.61 | 41.15 | 40.76 | 35.94 |
| ID12 | 44.60 | 43.06 | 36.45 | 58.70 | 43.53 | 59.39 | 42.93 | 49.07 | 36.45 |
| ID13 | 40.44 | 42.92 | 35.05 | 40.67 | 38.39 | 43.63 | 42.08 | 43.90 | 40.74 |
| ID14 | 40.27 | 32.89 | 34.92 | 36.44 | 41.07 | 39.48 | 38.86 | 42.33 | 31.43 |
| ID15 | 44.17 | 33.64 | 54.62 | 38.01 | 39.19 | 38.39 | 40.81 | 41.71 | 31.19 |
| ID16 | 42.27 | 72.35 | 35.15 | 45.45 | 41.24 | 45.36 | 38.10 | 38.99 | 64.57 |
| ID17 | 34.98 | 30.32 | 49.39 | 38.39 | 40.78 | 33.18 | 36.49 | 37.16 | 30.32 |
| ID18 | 40.09 | 40.98 | 32.33 | 36.12 | 34.77 | 39.06 | 38.68 | 39.69 | 38.21 |
| | ID10 | ID11 | ID12 | ID13 | ID14 | ID15 | ID16 | ID17 | ID18 |
| ID1 | 33.33 | 45.24 | 44.60 | 40.44 | 40.27 | 44.17 | 42.27 | 34.98 | 40.09 |
| ID2 | 43.91 | 42.72 | 43.06 | 42.92 | 32.89 | 33.64 | 72.35 | 30.32 | 40.98 |
| ID3 | 33.46 | 41.63 | 36.45 | 35.05 | 34.92 | 54.62 | 35.15 | 49.39 | 32.33 |
| ID4 | 39.41 | 43.05 | 58.70 | 40.67 | 36.44 | 38.01 | 45.45 | 38.39 | 36.12 |
| ID5 | 43.38 | 56.61 | 43.53 | 38.39 | 41.07 | 39.19 | 41.24 | 40.78 | 34.77 |
| ID6 | 38.85 | 43.61 | 59.39 | 43.63 | 39.48 | 38.39 | 45.36 | 33.18 | 39.06 |
| ID7 | 30.91 | 41.15 | 42.93 | 42.08 | 38.86 | 40.81 | 38.10 | 36.49 | 38.68 |
| ID8 | 35.75 | 40.76 | 49.07 | 43.90 | 42.33 | 41.71 | 38.99 | 37.16 | 39.69 |
| ID9 | 39.34 | 35.94 | 36.45 | 40.74 | 31.43 | 31.19 | 64.57 | 30.32 | 38.21 |
| ID10 | 100.00 | 34.63 | 34.78 | 39.29 | 31.39 | 29.68 | 48.66 | 30.56 | 38.35 |
| ID11 | 34.63 | 100.00 | 42.11 | 37.16 | 36.44 | 40.62 | 38.53 | 37.78 | 35.59 |
| ID12 | 34.78 | 42.11 | 100.00 | 43.69 | 39.55 | 40.85 | 46.38 | 40.93 | 35.27 |
| ID13 | 39.29 | 37.16 | 43.69 | 100.00 | 36.28 | 33.98 | 48.40 | 33.33 | 54.66 |
| ID14 | 31.39 | 36.44 | 39.55 | 36.28 | 100.00 | 40.42 | 33.64 | 37.66 | 30.34 |
| ID15 | 29.68 | 40.62 | 40.85 | 33.98 | 40.42 | 100.00 | 30.23 | 63.97 | 27.56 |
| ID16 | 48.66 | 38.53 | 46.38 | 48.40 | 33.64 | 30.23 | 100.00 | 31.82 | 38.70 |
| ID17 | 30.56 | 37.78 | 40.93 | 33.33 | 37.66 | 63.97 | 31.82 | 100.00 | 30.23 |
| ID18 | 38.35 | 35.59 | 35.27 | 54.66 | 30.34 | 27.56 | 38.70 | 30.23 | 100.00 |

This clearly illustrates how different the GH61 polypeptides tested in the present invention are.

In a preferred embodiment of the present invention a specific subset of GH61 polypeptides indicated in Table 2 may be used to enhance the enzyme detergency benefit of one or more enzymes.

TABLE 2

| SEQ | | | | | |
|---|---|---|---|---|---|
| ID | Mature | GH61 | | Short | |
| NO | protein | domain | Organism | name | Reference |
| 3 | 22-371 | 22-252 | Aspergillus terreus | At1 | UnitProt |
| | | | | | Q0CEG4 |
| 4 | 21-330 | 21-241 | Neurospora crassa | Nc1 | UniProt |
| | | | | | Q7RWN7 |
| 9 | 21-225 | 21-225 | Poronia punctata | Pp1 | |
| 10 | 20-298 | 20-236 | Humicola insolens | Hi2 | |
| 11 | 18-246 | 18-246 | Verticillium tenerum | Vt1 | |
| 12 | 17-234 | 17-234 | Verticillium tenerum | Vt2 | |
| 13 | 17-238 | 17-238 | Aspergillus terreus | At2 | UniProt Q0CDX1 |
| 14 | 21-259 | 21-259 | Chaetomium | Cg1 | |
| | | | globosum | | |
| 16 | 20-296 | 20-224 | Humicola insolens | Hi3 | |
| 17 | 20-248 | 20-248 | Aspergillus terreus | At3 | |
| 18 | 21-298 | 21-245 | Aspergillus terreus | At4 | UniProt Q0C7Z0 |

In another preferred embodiment of the present invention a specific subset of GH61 polypeptides indicated in Table 3 may be used to enhance the enzyme detergency benefit of one or more enzymes.

TABLE 3

| SEQ | Mature | GH61 | | |
|---|---|---|---|---|
| ID NO | protein | domain | Organism | Short name |
| 9 | 21-225 | 21-225 | Poronia punctata | Pp1 |
| 10 | 20-298 | 20-236 | Humicola insolens | Hi2 |
| 11 | 18-246 | 18-246 | Verticillium tenerum | Vt1 |
| 12 | 17-234 | 17-234 | Verticillium tenerum | Vt2 |
| 14 | 21-259 | 21-259 | Chaetomium globosum | Cg1 |
| 16 | 20-296 | 20-224 | Humicola insolens | Hi3 |
| 17 | 20-248 | 20-248 | Aspergillus terreus | At3 |

In a preferred embodiment the GH61 polypeptide applied in the uses of the present invention is a mature GH61 polypeptide or a functional fragment thereof, more preferably at least one of the mature GH61 polypeptides in table 1 to 3 is applied in the uses of the present invention or a functional fragment thereof, even more preferably a polypeptide comprising at least one of the GH61 domains in table 1 to 3 is applied in the uses of the present invention, or functional fragment of thereof.

In addition to the uses of the GH61 polypeptides in table 1 to 3 the present invention encompasses applying a GH61 polypeptide variant comprising an amino acid sequence which has at least 70% identity to one of the GH61 polypeptides in table 1, 2 or 3, preferably at least 70%, 75%, 80%, 85%, 90%, 91%, 92%, 95%, 96%, 97%, 98% or 99% identity to one of these GH61 polypeptides or functional fragment thereof, in the uses of the present invention. GH61 polypeptide variants and functional fragments thereof should still fall within definition of a GH61 polypeptide. Preferably, such a GH61 polypeptide variant or fragment maintain at least 20%, preferably at least 40%, more preferably at least 50%, more preferably at least 60%, more preferably at least 70%, more preferably at least 80%, even more preferably at least 90%, most preferably at least 95%, and even most preferably at least 100% of the enzyme detergency enhancing effect of the GH61 polypeptide in Table 1 to which it has the highest identity. Alternatively, such a GH61 polypeptide variant or fragment maintain at least 20%, preferably at least 40%, more preferably at least 50%, more preferably at least 60%, more preferably at least 70%, more preferably at least 80%, even more preferably at least 90%, most preferably at least 95%, and even most preferably at least 100% of the pyrogallol activity of the GH61 polypeptide in Table 1 to which it has the highest identity.

In the present invention the GH61 is preferably used in a range from 0.025 to 1.5 mg/L, more preferably in the range of 0.05 to 1 mg/L, more preferably in the range of 0.1 to 0.75 mg/L, more preferably from 0.15 to 0.5 mg/L most preferably from 0.2 to 0.4 mg/L. Where mg refers to pure polypeptide and L refers to the volume of the solution in which the polypeptide is used, e.g. wash solution.

The present invention furthermore relates to novel isolated GH61 polypeptides having an amino acid sequence which has a degree of identity to amino acid residues 21 to 225 of SEQ ID NO:9, amino acid residues 20 to 298 of SEQ ID NO:10, amino acid residues 18 to 246 of SEQ ID NO:11, amino acid residues 17 to 234 of SEQ ID NO:12, amino acid residues 21 to 259 of SEQ ID NO:14, amino acid residues 20 to 296 of SEQ ID NO:16, or amino acid residues 20 to 248 of SEQ ID NO:17 (i.e., the mature polypeptide) of at least 70%, preferably at least 75%, more preferably at least 80%, more preferably at least 85%, even more preferably at least 90%, most preferably at least 95%, and even most preferably at least 97%, 98%, or 99 which can still be classified as a GH61 and/or which has enzyme detergency enhancing effect (hereinafter “homologous polypeptides”). In a preferred aspect, the homologous polypeptides have an amino acid sequence which differs by ten amino acids, preferably by five amino acids, more preferably by four amino acids, even more preferably by three amino acids, most preferably by two amino acids, and even most preferably by one amino acid from amino acids to amino acid residues 21 to 225 of SEQ ID NO:9, amino acid residues 20 to 298 of SEQ ID NO:10, amino acid residues 18 to 246 of SEQ ID NO:11, amino acid residues 17 to 234 of SEQ ID NO:12, amino acid residues 21 to 259 of SEQ ID NO:14, amino acid residues 20 to 296 of SEQ ID NO:16, or amino acid residues 20 to 248 of SEQ ID NO:17.

In a first aspect, the present invention relates to an isolated GH61polypeptide from Poronia punctata. Preferably, the isolated polypeptide comprises an amino acid sequence having a degree of sequence identity to the mature polypeptide of SEQ ID NO: 9 of preferably at least 75%, more preferably at least 80%, more preferably at least 85%, even more preferably at least 90%, most preferably at least 95%, and even most preferably at least 96%, at least 97%, at least 98%, or at least 99%, which can still be classified as a GH61 and/or which has enzyme detergency enhancing effect (hereinafter “homologous polypeptides. In a preferred aspect, the polypeptide comprises the amino acid sequence of SEQ ID NO: 9. In another preferred aspect, the polypeptide comprises the mature polypeptide of SEQ ID NO: 9. In another preferred aspect, the polypeptide comprises amino acids 21 to 225 of SEQ ID NO: 9, or an allelic variant thereof; or a functional fragment thereof. In another preferred aspect, the polypeptide consists of the amino acid sequence of SEQ ID NO: 9 or an allelic variant thereof; or a functional fragment thereof. In another preferred aspect, the polypeptide consists of the mature polypeptide of SEQ ID NO: 9. In another preferred aspect, the polypeptide consists of amino acids 21 to 225 of SEQ ID NO: 9 or an allelic variant thereof, or a functional fragment thereof.

In a second aspect, the present invention relates to an isolated GH61 polypeptide from Humicola insolens. Preferably, the isolated polypeptide comprises an amino acid sequence having a degree of sequence identity to the mature polypeptide of SEQ ID NO: 10 of preferably at least 85%, more preferably at least 90%, even more preferably at least 95%, and most preferably at least 96%, at least 97%, at least 98%, or at least 99%, which can still be classified as a GH61 and/or which has enzyme detergency enhancing effect or a degree of sequence identity to the mature polypeptide of SEQ ID NO: 16 of preferably at least 80%, more preferably at least 85%, even more preferably at least 90%, most preferably at least 95%, and even most preferably at least 96%, at least 97%, at least 98%, or at least 99%, which can still be classified as a GH61 and/or which has enzyme detergency enhancing effect (hereinafter “homologous polypeptides”). In a preferred aspect, the polypeptide comprises the amino acid sequence of SEQ ID NO: 10 or SEQ ID NO: 11. In another preferred aspect, the polypeptide comprises the mature polypeptide of SEQ ID NO: 10 or SEQ ID NO: 16. In another preferred aspect, the polypeptide comprises amino acids 20 to 236 of SEQ ID NO: 10 or amino acids 20 to 224 of SEQ ID NO:16, or an allelic variant thereof; or a functional fragment thereof. In another preferred aspect, the polypeptide consists of the amino acid sequence of SEQ ID NO: 10 or SEQ ID NO: 16 or an allelic variant thereof; or a functional fragment thereof. In another preferred aspect, the polypeptide consists of the mature polypeptide of SEQ ID NO: 10 or SEQ ID NO: 16. In another preferred aspect, the polypeptide consists of amino acids 20 to 236 of SEQ ID NO: 10 or amino acids 20 to 224 of SEQ ID NO: 16 or an allelic variant thereof, or a functional fragment thereof.

In a third aspect, the present invention relates to an isolated GH61polypeptide from Verticillium tenerum. Preferably, the isolated polypeptide comprises an amino acid sequence having a degree of sequence identity to the mature polypeptide of SEQ ID NO: 11 or SEQ ID NO: 12 of preferably at least preferably at least 70%, more preferably at least 75%, more preferably at least 80%, more preferably at least 85%, even more preferably at least 90%, most preferably at least 95%, and even most preferably at least 96%, at least 97%, at least 98%, or at least 99%, which can still be classified as a GH61 and/or which has enzyme detergency enhancing effect (hereinafter “homologous polypeptides”). In a preferred aspect, the polypeptide comprises the amino acid sequence of SEQ ID NO: 11 or SEQ ID NO: 12. In another preferred aspect, the polypeptide comprises the mature polypeptide of SEQ ID NO: 11 or SEQ ID NO: 12. In another preferred aspect, the polypeptide comprises amino acids 18 to 246 of SEQ ID NO: 11 or amino acids 17 to 234 of SEQ ID NO: 12, or an allelic variant thereof; or a functional fragment thereof. In another preferred aspect, the polypeptide consists of the amino acid sequence of SEQ ID NO: 11 or SEQ ID NO: 12 or an allelic variant thereof; or a functional fragment thereof. In another preferred aspect, the polypeptide consists of the mature polypeptide SEQ ID NO: 11 or SEQ ID NO: 12. In another preferred aspect, the polypeptide consists of amino acids 18 to 246 of SEQ ID NO: 11 or amino acids 17 to 234 of SEQ ID NO: 12 or an allelic variant thereof, or a functional fragment thereof.

In a fourth aspect, the present invention relates to an isolated GH61 polypeptide from Chaetomium globosum. Preferably, the isolated polypeptide comprises an amino acid sequence having a degree of sequence identity to the mature polypeptide of SEQ ID NO: 14 of preferably at least 95%, more preferably at least 96%, even more preferably at least 97%, most preferably at least 98%, and even most preferably at least 99%, which can still be classified as a GH61 and/or which has enzyme detergency enhancing effect (hereinafter “homologous polypeptides”). In a preferred aspect, the polypeptide comprises the amino acid sequence of SEQ ID NO: 14. In another preferred aspect, the polypeptide comprises the mature polypeptide of SEQ ID NO: 14. In another preferred aspect, the polypeptide comprises amino acids 21 to 259 of SEQ ID NO: 14, or an allelic variant thereof; or a functional fragment thereof. In another preferred aspect, the polypeptide consists of the amino acid sequence of SEQ ID NO: 14 or an allelic variant thereof; or a functional fragment thereof. In another preferred aspect, the polypeptide consists of the mature polypeptide of SEQ ID NO: 14. In another preferred aspect, the polypeptide consists of amino acids 21 to 259 of SEQ ID NO: 14 or an allelic variant thereof, or a functional fragment thereof.

In a fifth aspect, the present invention relates to an isolated GH61polypeptide from Aspergillus terreus. Preferably, the isolated polypeptide comprises an amino acid sequence having a degree of sequence identity to the mature polypeptide of SEQ ID NO: 17 of preferably at least 97.5%, more preferably at least 98%, and even most preferably at least 99%, which can still be classified as a GH61 and/or which has enzyme detergency enhancing effect (hereinafter “homologous polypeptides”). In a preferred aspect, the polypeptide comprises the amino acid sequence of SEQ ID NO: 17. In another preferred aspect, the polypeptide comprises the mature polypeptide of SEQ ID NO: 17. In another preferred aspect, the polypeptide comprises amino acids 20 to 248 of SEQ ID NO: 17, or an allelic variant thereof; or a functional fragment thereof. In another preferred aspect, the polypeptide consists of the amino acid sequence of SEQ ID NO: 17 or an allelic variant thereof; or a functional fragment thereof. In another preferred aspect, the polypeptide consists of the mature polypeptide of SEQ ID NO: 17. In another preferred aspect, the polypeptide consists of amino acids 20 to 248 of SEQ ID NO: 17 or an allelic variant thereof, or a functional fragment thereof.

In a further aspect, the present invention relates to an isolated GH61polypeptide from Thermoascus aurantiaticus. Preferably, the isolated polypeptide comprises the amino acid sequence of SEQ ID NO: 15. In another preferred aspect, the polypeptide comprises the mature polypeptide of SEQ ID NO: 15. In another preferred aspect, the polypeptide comprises amino acids 22 to 249 of SEQ ID NO: 15, or an allelic variant thereof; or a functional fragment thereof. In another preferred aspect, the polypeptide consists of the amino acid sequence of SEQ ID NO: 15 or an allelic variant thereof; or a functional fragment thereof. In another preferred aspect, the polypeptide consists of the mature polypeptide of SEQ ID NO: 15. In another preferred aspect, the polypeptide consists of amino acids 22 to 249 of SEQ ID NO: 15 or an allelic variant thereof; or a functional fragment thereof.

The present invention furthermore relates to isolated polypeptides classified as a GH61 and/or which have enzyme detergency enhancing effect which are encoded by polynucleotides which hybridize under low stringency conditions, more preferably medium stringency conditions, more preferably medium-high stringency conditions, even more preferably high stringency conditions, and most preferably very high stringency conditions with (i) nucleotides 126 to 740 of SEQ ID NO: 19, nucleotides 97 to 933 of SEQ ID NO: 20, nucleotides 111 to 797 of SEQ ID NO: 21, nucleotides 109 to 762 of SEQ ID NO: 22, nucleotides 61 to 777 of SEQ ID NO: 23, nucleotides 155 to 985 of SEQ ID NO: 24 or nucleotides 57 to 744 of SEQ ID NO: 25 (ii) the cDNA sequence contained in nucleotides 126 to 740 of SEQ ID NO: 19, nucleotides 97 to 933 of SEQ ID NO: 20, nucleotides 111 to 797 of SEQ ID NO: 21, nucleotides 109 to 762 of SEQ ID NO: 22, nucleotides 61 to 777 of SEQ ID NO: 23, nucleotides 155 to 985 of SEQ ID NO: 24 or nucleotides 57 to 744 of SEQ ID NO: 25, (iii) a subsequence of (i) or (ii), or (iv) a complementary strand of (i), (ii), or (iii) (J. Sambrook, E. F. Fritsch, and T. Maniatus, 1989, Molecular Cloning, A Laboratory Manual, 2d edition, Cold Spring Harbor, N.Y.). A subsequence of SEQ ID NO: 19, 20, 21, 22, 23, 24, or 25 contains at least 100 contiguous nucleotides or preferably at least 200 contiguous nucleotides. Moreover, the subsequence may encode a polypeptide fragment which has enzyme detergency enhancing effect.

The nucleotide sequence of SEQ ID NO: 19, 20, 21, 22, 23, 24, or 25; or a subsequence thereof; as well as the amino acid sequence of SEQ ID NO: 9, 10, 11, 12, 14, 16 or 17; or a functional fragment thereof, may be used to design a nucleic acid probe to identify and clone DNA encoding polypeptides having enzyme detergency enhancing effect from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes which are at least 600 nucleotides, at least preferably at least 700 nucleotides, more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other organisms may, therefore, be screened for DNA which hybridizes with the probes described above and which encodes a polypeptide having enzyme detergency enhancing effect. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with SEQ ID NO: 19, 20, 21, 22, 23, 24, or 25 or a subsequence thereof, the carrier material is used in a Southern blot.

For purposes of the present invention, hybridization indicates that the nucleotide sequence hybridizes to a labeled nucleic acid probe corresponding to the nucleotide sequence shown in SEQ ID NO: 19, 20, 21, 22, 23, 24, or 25, its complementary strand, or a subsequence thereof, under low to very high stringency conditions. Molecules to which the nucleic acid probe hybridizes under these conditions can be detected using X-ray film.

In a preferred embodiment, the nucleic acid probe is a nucleic acid sequence which encodes the polypeptide of SEQ ID NO: 9, or a subsequence thereof. In another preferred embodiment, the nucleic acid probe is SEQ ID NO: 19. In another preferred embodiment, the nucleic acid probe is the mature polypeptide coding region of SEQ ID NO: 19.

In another preferred embodiment, the nucleic acid probe is a nucleic acid sequence which encodes the polypeptide of SEQ ID NO: 10, or a subsequence thereof for example as indicated in Table 1. In another preferred embodiment, the nucleic acid probe is SEQ ID NO: 20. In another preferred embodiment, the nucleic acid probe is the mature polypeptide coding region of SEQ ID NO: 20.

In another preferred embodiment, the nucleic acid probe is a nucleic acid sequence which encodes the polypeptide of SEQ ID NO: 11, or a subsequence thereof for example as indicated in Table 1. In another preferred embodiment, the nucleic acid probe is SEQ ID NO: 21. In another preferred embodiment, the nucleic acid probe is the mature polypeptide coding region of SEQ ID NO: 21.

In another preferred embodiment, the nucleic acid probe is a nucleic acid sequence which encodes the polypeptide of SEQ ID NO: 12, or a subsequence thereof for example as indicated in Table 1. In another preferred embodiment, the nucleic acid probe is SEQ ID NO: 22. In another preferred embodiment, the nucleic acid probe is the mature polypeptide coding region of SEQ ID NO: 22. In another preferred embodiment, the nucleic acid probe is the nucleic acid sequence contained

In another preferred embodiment, the nucleic acid probe is a nucleic acid sequence which encodes the polypeptide of SEQ ID NO: 14, or a subsequence thereof for example as indicated in Table 1. In another preferred embodiment, the nucleic acid probe is SEQ ID NO: 23. In another preferred embodiment, the nucleic acid probe is the mature polypeptide coding region of SEQ ID NO: 23.

In another preferred embodiment, the nucleic acid probe is a nucleic acid sequence which encodes the polypeptide of SEQ ID NO: 16, or a subsequence thereof for example as indicated in Table 1. In another preferred embodiment, the nucleic acid probe is SEQ ID NO: 24. In another preferred embodiment, the nucleic acid probe is the mature polypeptide coding region of SEQ ID NO: 24.

In another preferred embodiment, the nucleic acid probe is a nucleic acid sequence which encodes the polypeptide of SEQ ID NO: 17, or a subsequence thereof for example as indicated in Table 1. In another preferred embodiment, the nucleic acid probe is SEQ ID NO: 25. In another preferred embodiment, the nucleic acid probe is the mature polypeptide coding region of SEQ ID NO: 25.

For long probes of at least 100 nucleotides in length, low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₙ, using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Substantially homologous polypeptides of the sequences described above (GH61 polypeptide variants) are characterized as having one or more (several) amino acid a substitutions, deletions, and/or insertions in the mature polypeptide. Preferably, amino acid changes are of a minor nature, that is conservative amino acid substitutions or insertions that do not significantly affect the folding and/or activity of the protein; small deletions, typically of one to about 9 amino acids, preferably from one to about 15 amino acids and most preferably from one to about 30 amino acids; small amino- or carboxyl-terminal extensions, such as an amino-terminal methionine residue; a small linker peptide of up to about five to ten residues, preferably from 10 to 15 residues and most preferably from 20 to 25 residues, or a small extension that facilitates purification by changing net charge or another function, such as a poly-histidine tag, an antigenic epitope, protein A, a CBM or a another binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., enzyme detergency enhancing effects or pyrogallol activity) to identify amino acid residues that are critical to the activity of the molecule. See also, Hilton et al., 1996, J. Biol. Chem. 271: 4699-4708. Three dimensional structures, such as alpha-helixes, beta-sheets, as well as metal binding site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. Especially, Karkehabadi et al., 2008 J. Mol. Biol. 383: 144-154 describes the crystal structure of GH61 from Hypocrea jecorina and Harris et al, 2010, Biochem, 49:3305-3316 describes the crystal structure of GH61E from Thielavia terrestris (equivalent to Tt5 of the present invention). The identities of essential amino acids can also be inferred from analysis of identities with polypeptides that are related to a polypeptide according to the invention.

GH61 polypeptides are identified by their consensus sequence motif: [ILMV]-[QP]-x(4,5)-[AGS]-x-Y-[ILMV]-x-R-x-[EQ]-x(4)-[EHNQST]. The respective consensus residues corresponds to positions 141 to 160 in SEQ ID NO: 8, SEQ ID NO: 2, SEQ ID NO: 9, positions 147 to 176 in SEQ ID NO: 1, positions 166 to 185 in SEQ ID NO: 3, position 160 to 179 in SEQ ID NO: 4 and SEQ ID NO: 6, position 152 to 171 in SEQ ID NO: 5, position 153 to 172 in SEQ ID NO: 7, Position 151 to 170 in SEQ ID NO: 10 and SEQ ID NO: 12, position 145 to 174 in SEQ ID NO: 11 and SEQ ID No: 13, position 162 to 181 in SEQ ID No 14, position 166 to 185 in SEQ ID NO: 15, position 140 to 159 in SEQ ID NO:16, position 164 to 183 in SEQ ID NO: 17 and position 161 to 180 in SEQ ID NO: 18. In a preferred embodiment all consensus positions corresponding to positions 141, 142, 148, 150, 151, 153, 155 and 160 (numbering according to SEQ ID NO: 8) are present in a GH61 polypeptide of the present invention. The positions in other GH61 polypeptides which correspond to the SEQ ID NO: 8 numbering can be identified by alignment with SEQ ID NO: 8.

The amino acid residues in position 19 corresponding to the N-terminal histidine of the mature polypeptide and position 86 (SEQ ID NO: 8 numbering) are predicted to be directly involved in metal binding and are important for the activity. In a preferred embodiment of the present invention the GH61 polypeptide contains a histidine in position 19 and a histidine or a glutamine in position 86 (using SEQ ID NO: 8 numbering).

Positions 85, 209 and 210 (SEQ ID NO: 8 numbering) are potentially involved in polysaccharide binding. In a preferred embodiment of the present invention the GH61 polypeptide contains a polar amino acid in position 85, and an aromatic amino acid, preferably a tyrosine, tryptophan or histidine in position 209 (using SEQ ID NO: 8 numbering).

Position 153 and 155 (SEQ ID NO: 8 numbering) participate in a ionic network important for GH61 activity. In a preferred embodiment of the present invention the GH61 polypeptide contains a argentine in position 153, and glutamic acid or glutamine in position 155 (using SEQ ID NO: 8 numbering).

Position 56 and 174 (SEQ ID NO: 8 numbering) are predicted to engage in a cysteine bridge and may therefore be important for the stability of GH61 polypeptides. In a preferred embodiment of the present invention the GH61 polypeptide contains a cysteine in position 56, and 174 (using SEQ ID NO: 8 numbering).

The amino acids in position 169 and 171 (SEQ ID NO: 8 numbering) may form an important hydrogen bound and it has been shown that mutations which disturb this hydrogen bound decrease the GH61 activity (Harris et al, 2010, Biochem, 49:3305-3316). In a preferred embodiment of the present invention the GH61 polypeptide contains a glutamine, glutamic acid or aspargine in position 169, and a tyrosine or a phenylalanine in position 171 (using SEQ ID NO: 8 numbering).

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

### Sources of Polypeptides Having Enzyme Detergency Enhancing Effect

A GH61 polypeptide useful in the present invention may be obtained from microorganisms of any genus. For purposes of the present invention, the term “obtained from” as used herein in connection with a given source shall mean that the polypeptide encoded by a nucleotide sequence is produced by the source in which it is naturally present or by a strain in which the nucleotide sequence from the source has been inserted. In a preferred aspect, the polypeptide obtained from a given source is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

A polypeptide of the present invention may also be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Chaetomium, Cryptococcus, Filibasidium, Fusarium, Humicola, Magnaporthe, Mucor, Myceliophthora, Neocallimastix, Neurospora, Paecilomyces, Penicillium, Piromyces, Poronia, Schizophyllum, Talaromyces, Thermoascus, Thielavia, Tolypocladium, Trichoderma or Verticillium polypeptide.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme detergency enhancing effect.

In another preferred aspect, the polypeptide is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus terreus, Chaetomium globosum, Coprinus cinereus, Diplodia gossyppina, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Magnaporthe grisea, Mucor miehei, Myceliophthora thermophila, Neurospora crassa, Penicillium purpurogenum, Phanerochaete chrysosporium, Poronia punctata, Pseudoplectania nigrella, Thermoascus aurantiacus, Thielavia terrestris, Trichoderma harzianum, Trichoderma koningii, Trichoderma longibrachiatum, Trichoderma reesei, Trichoderma viride, Trichophaea saccata or Verticillium tenerum polypeptide.

In a more preferred aspect, the polypeptide is an Aspergillus terreus ATCC28865, Chaetomium globosum CBS148.51, Humicola insolens DSM1800, Poronia punctata CBS 417.94, or Verticillium tenerum CBS109513 polypeptide.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of such a microorganism. Once a polynucleotide sequence encoding a polypeptide has been detected with the probe(s), the polynucleotide can be isolated or cloned by utilizing techniques which are well known to those of ordinary skill in the art (see, e.g., Sambrook et al., 1989, supra).

Polypeptides of the present invention also include fused polypeptides or cleavable fusion polypeptides in which another polypeptide is fused at the N-terminus or the C-terminus of the polypeptide or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

### Polynucleotides

The present invention also relates to isolated polynucleotides having nucleotide sequences which encode polypeptides of the present invention.

In a preferred aspect, the nucleotide sequence is set forth in SEQ ID NO: 19, preferably nucleotides 66 to 740 of SEQ ID NO: 19, even more preferably nucleotides 126 to 740 of SEQ ID NO: 19. In another preferred aspect, the nucleotide sequence is the mature polypeptide coding region of SEQ ID NO: 19. The present invention also encompasses nucleotide sequences which encode a polypeptide having the amino acid sequence of SEQ ID NO: 9 or the mature polypeptide thereof, which differs from SEQ ID NO: 19 by virtue of the degeneracy of the genetic code. The present invention also relates to subsequences of SEQ ID NO: 19 which encode functional fragments of SEQ ID NO: 9 that have enzyme detergency enhancing effect.

In another preferred aspect, the nucleotide sequence is set forth in SEQ ID NO: 20, preferably nucleotides 40 to 933 of SEQ ID NO: 20, even more preferably nucleotides 97 to 933 of SEQ ID NO: 20. In another preferred aspect, the nucleotide sequence is the mature polypeptide coding region of SEQ ID NO: 20. The present invention also encompasses nucleotide sequences which encode a polypeptide having the amino acid sequence of SEQ ID NO: 10 or the mature polypeptide thereof, which differs from SEQ ID NO: 20 by virtue of the degeneracy of the genetic code. The present invention also relates to subsequences of SEQ ID NO: 20 which encode functional fragments of SEQ ID NO: 10 that have enzyme detergency enhancing effect.

In another preferred aspect, the nucleotide sequence is set forth in SEQ ID NO: 21, preferably nucleotides 60 to 797 of SEQ ID NO: 21, even more preferably nucleotides 111 to 797 of SEQ ID NO: 21. In another preferred aspect, the nucleotide sequence is the mature polypeptide coding region of SEQ ID NO: 21. The present invention also encompasses nucleotide sequences which encode a polypeptide having the amino acid sequence of SEQ ID NO: 11 or the mature polypeptide thereof, which differs from SEQ ID NO: 21 by virtue of the degeneracy of the genetic code. The present invention also relates to subsequences of SEQ ID NO: 11 which encode functional fragments of SEQ ID NO: 21 that have enzyme detergency enhancing effect.

In another preferred aspect, the nucleotide sequence is set forth in SEQ ID NO: 22, preferably nucleotides 61 to 762 of SEQ ID NO: 22, even more preferably nucleotides 109 to 762 of SEQ ID NO: 20. In another preferred aspect, the nucleotide sequence is the mature polypeptide coding region of SEQ ID NO: 22. The present invention also encompasses nucleotide sequences which encode a polypeptide having the amino acid sequence of SEQ ID NO: 12 or the mature polypeptide thereof, which differ from SEQ ID NO: 22 by virtue of the degeneracy of the genetic code. The present invention also relates to subsequences of SEQ ID NO: 22 which encode functional fragments of SEQ ID NO: 12 that have enzyme detergency enhancing effect.

In another preferred aspect, the nucleotide sequence is set forth in SEQ ID NO: 23, preferably nucleotides 61 to 777 of SEQ ID NO: 23. In another preferred aspect, the nucleotide sequence is the mature polypeptide coding region of SEQ ID NO: 23. The present invention also encompasses nucleotide sequences which encode a polypeptide having the amino acid sequence of SEQ ID NO: 14 or the mature polypeptide thereof, which differs from SEQ ID NO: 23 by virtue of the degeneracy of the genetic code. The present invention also relates to subsequences of SEQ ID NO: 23 which encode fragments of SEQ ID NO: 14 that have enzyme detergency enhancing effect.

In another preferred aspect, the nucleotide sequence is set forth in SEQ ID NO: 24, preferably nucleotides 98 to 985 of SEQ ID NO: 24, even more preferably nucleotides 155 to 985 of SEQ ID NO: 24. In another preferred aspect, the nucleotide sequence is the mature polypeptide coding region of SEQ ID NO: 24. The present invention also encompasses nucleotide sequences which encode a polypeptide having the amino acid sequence of SEQ ID NO: 16 or the mature polypeptide thereof, which differs from SEQ ID NO: 24 by virtue of the degeneracy of the genetic code. The present invention also relates to subsequences of SEQ ID NO: 24 which encode fragments of SEQ ID NO: 16 that have enzyme detergency enhancing effect.

In another preferred aspect, the nucleotide sequence is set forth in SEQ ID NO: 25, preferably nucleotides 57 to 744 of SEQ ID NO: 25. In another preferred aspect, the nucleotide sequence is the mature polypeptide coding region of SEQ ID NO: 25. The present invention also encompasses nucleotide sequences which encode a polypeptide having the amino acid sequence of SEQ ID NO: 17 or the mature polypeptide thereof, which differs from SEQ ID NO: 25 by virtue of the degeneracy of the genetic code. The present invention also relates to subsequences of SEQ ID NO: 25 which encode fragments of SEQ ID NO: 17 that have enzyme detergency enhancing effect.

The techniques used to isolate or clone a polynucleotide encoding a polypeptide are known in the art and include isolation from genomic DNA, preparation from cDNA, or a combination thereof. The cloning of the polynucleotides of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used. The polynucleotides may be cloned from a strain of Aspergillus terreus, Chaetomium globosum, Humicola insolens, Poronia punctata, or Verticillium tenerum, or another or related organism as indicated in the section “Sources of Polypeptides Having enzyme detergency enhancing effect” and thus, for example, may be an allelic or species variant of the polypeptide encoding region of the nucleotide sequence.

The present invention also relates to polynucleotides having nucleotide sequences which have a degree of identity to the mature polypeptide coding sequence of SEQ ID NO: 19 (i.e., nucleotides 126 to 740 of at least 75%, preferably at least 80%, more preferably at least 85%, even more preferably at least 90%, most preferably at least 95%, most preferably at least 97% identity and even most preferably 98% or 99% identity, which encode an active polypeptide.

The present invention also relates to polynucleotides having nucleotide sequences which have a degree of identity to the mature polypeptide coding sequence of SEQ ID NO: 20 (i.e., nucleotides 97 to 933) of at least 75%, preferably at least 80%, more preferably at least 85%, even more preferably at least 90%, most preferably at least 95%, most preferably at least 97% identity and even most preferably 98% or 99% identity, which encode an active polypeptide.

The present invention also relates to polynucleotides having nucleotide sequences which have a degree of identity to the mature polypeptide coding sequence of SEQ ID NO: 21 (i.e., nucleotides 111 to 797) of at least 75%, preferably at least 80%, more preferably at least 85%, even more preferably at least 90%, most preferably at least 95%, preferably at least 97% identity and even most preferably 98% or 99% identity, which encode an active polypeptide.

The present invention also relates to polynucleotides having nucleotide sequences which have a degree of identity to the mature polypeptide coding sequence of SEQ ID NO: 22 (i.e., nucleotides 109 to 762) of at least 75%, preferably at least 80%, more preferably at least 85%, even more preferably at least 90%, most preferably at least 95%, most preferably at least 97% identity and even most preferably 98% or 99% identity, which encode an active polypeptide.

The present invention also relates to polynucleotides having nucleotide sequences which have a degree of identity to the mature polypeptide coding sequence of SEQ ID NO: 23 (i.e., nucleotides 61 to 777) of at least 75%, preferably at least 80%, more preferably at least 85%, even more preferably at least 90%, most preferably at least 95%, most preferably at least 97% identity and even most preferably 98% or 99% identity, which encode an active polypeptide.

The present invention also relates to polynucleotides having nucleotide sequences which have a degree of identity to the mature polypeptide coding sequence of SEQ ID NO: 24 (i.e., nucleotides 155 to 985) of at least 75%, preferably at least 80%, more preferably at least 85%, even more preferably at least 90%, most preferably at least 95%, most preferably at least 97% identity, and even most preferably 98% or 99% identity which encode an active polypeptide.

The present invention also relates to polynucleotides having nucleotide sequences which have a degree of identity to the mature polypeptide coding sequence of SEQ ID NO: 25 (i.e., nucleotides 57 to 744) of at least 75%, preferably at least 80%, more preferably at least 85%, even more preferably at least 90%, most preferably at least 95%, most preferably at least 97% identity and even most preferably 98% or 99% identity, which encode an active polypeptide.

Modification of a nucleotide sequence encoding a polypeptide of the present invention may be necessary for the synthesis of polypeptides substantially similar to the polypeptide. The term “substantially similar” to the polypeptide refers to non-naturally occurring forms of the polypeptide. These polypeptides may differ in some engineered way from the polypeptide isolated from its native source, e.g., artificial variants that differ in specific activity, thermostability, pH optimum, or the like. The variant sequence may be constructed on the basis of the nucleotide sequence presented as the polypeptide encoding region of SEQ ID NO: 19, 20, 21, 22, 23, 24 or 25, e.g., a subsequence thereof, and/or by introduction of nucleotide substitutions which do not give rise to another amino acid sequence of the polypeptide encoded by the nucleotide sequence, but which correspond to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active polypeptide. Amino acid residues essential to the activity of the polypeptide encoded by an isolated polynucleotide of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for enzyme detergency enhancing effect to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labeling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated polynucleotides encoding a polypeptide of the present invention, which hybridize under low stringency conditions, more preferably medium stringency conditions, more preferably medium-high stringency conditions, even more preferably high stringency conditions, and most preferably very high stringency conditions with (i) nucleotides 126 to 740 of SEQ ID NO: 19, nucleotides 97 to 933 of SEQ ID NO: 20, nucleotides 111 to 979 of SEQ ID NO: 21, nucleotides 109 to 762 of SEQ ID NO: 22, nucleotides 61 to 777 of SEQ ID NO: 23, nucleotides 155 to 985 of SEQ ID NO: 24 or nucleotides 57 to 744 of SEQ ID NO: 25, (ii) the cDNA sequence contained in nucleotides 126 to 740 of SEQ ID NO: 19, nucleotides 97 to 933 of SEQ ID NO: 20, nucleotides 111 to 979 of SEQ ID NO: 21, nucleotides 109 to 762 of SEQ ID NO: 22, nucleotides 61 to 777 of SEQ ID NO: 23, nucleotides 155 to 985 of SEQ ID NO: 24 or nucleotides 57 to 744 of SEQ ID NO: 25, or (iii) a complementary strand of (i) or (ii); or allelic variants and subsequences thereof (Sambrook et al., 1989, supra), as defined herein.

As will be understood, details and particulars concerning hybridization of the nucleotide sequences will be the same or analogous to the hybridization aspects discussed in the section titled “GH-61 polypeptides” herein.

### Nucleic Acid Constructs

The present invention also relates to nucleic acid constructs comprising an isolated polynucleotide of the present invention operably linked to one or more control sequences which direct the expression of the coding sequence in a suitable host cell under conditions compatible with the control sequences.

An isolated polynucleotide encoding a polypeptide of the present invention may be manipulated in a variety of ways to provide for expression of the polypeptide. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence which is recognized by a host cell for expression of a polynucleotide encoding a polypeptide of the present invention. The promoter sequence contains transcriptional control sequences which mediate the expression of the polypeptide. The promoter may be any nucleotide sequence which shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1,ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA which is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a signal peptide coding region that codes for an amino acid sequence linked to the amino terminus of a polypeptide and directs the encoded polypeptide into the cell's secretory pathway. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to the coding sequence. The foreign signal peptide coding region may be required where the coding sequence does not naturally contain a signal peptide coding region. Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice, i.e., secreted into a culture medium, may be used in the present invention.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis betalactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding regions for filamentous fungal host cells are the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleic acids and control sequences described herein may be joined together to produce a recombinant expression vector which may include one or more convenient restriction sites to allow for insertion or substitution of the nucleotide sequence encoding the polypeptide at such sites. Alternatively, a nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors of the present invention preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention, which are advantageously used in the recombinant production of the polypeptides. A vector comprising a polynucleotide of the present invention is introduced into a host cell so that the vector is maintained as a chromosomal integrant or as a self-replicating extrachromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a prokaryote such as bacterial cells, an archaea or an eukaryote such as fungal cells, plant cells, insect cells, or mammalian cells.

Useful prokaryotes are bacterial cells such as gram positive bacteria including, but not limited to, a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred aspect, the bacterial host cell is a Bacillus lentus, Bacillus licheniformis, Bacillus stearothermophilus, or Bacillus subtilis cell. In another preferred aspect, the Bacillus cell is an alkalophilic Bacillus.

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveti, Saccharomyces norbensis, or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chtysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present invention also relates to methods for producing a polypeptide of the present invention, comprising (a) cultivating a cell, which in its wild-type form is capable of producing the polypeptide, under conditions conducive for production of the polypeptide; and (b) recovering the polypeptide. Preferably, the cell is of the genus Aspergillus, Chaetomium, Humicola, Poronia or Verticillium and more preferably of the species Aspergillus terreus, Chaetomium globosum, Humicola insolens, Poronia punctata, or Verticillium tenerum.

The present invention also relates to methods for producing a polypeptide of the present invention, comprising (a) cultivating a host cell under conditions conducive for production of the polypeptide; and (b) recovering the polypeptide. Preferably, the host cell is a recombinant host cell comprising an expression vector of the present invention.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted into the medium, it can be recovered from cell lysates.

The polypeptides having enzyme detergency enhancing effect are detected using the methods described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

### Compositions

The present invention also relates to compositions comprising a GH61 polypeptide of the present invention and a carrier and/or an exhibient. In a preferred embodiment the composition comprises a glycosyl hydrolase family 61 polypeptide, selected from the group consisting of:
a) a polypeptide comprising an amino acid sequence from amino acid residues 21 to 225 of SEQ ID NO:9;
b) a polypeptide with at least 75% identity with the amino acid sequence from amino acid residues 21 to 225 of SEQ ID NO:9;
c) a polypeptide comprising an amino acid sequence from amino acid residues 20 to 298 of SEQ ID NO:10;
d) a polypeptide with at least 85% identity with the amino acid sequence from amino acid residues 20 to 298 of SEQ ID NO:10;
e) a polypeptide comprising an amino acid sequence from amino acid residues 18 to 246 of SEQ ID NO:11;
f) a polypeptide with at least 70% identity with the amino acid sequence from amino acid residues 18 to 246 of SEQ ID NO:11;
g) a polypeptide comprising an amino acid sequence from amino acid residues 17 to 234 of SEQ ID NO:12;
h) a polypeptide with at least 70% identity with the amino acid sequence from amino acid residues 17 to 234 of SEQ ID NO:12;
i) a polypeptide comprising an amino acid sequence from amino acid residues 21 to 259 of SEQ ID NO:14;
j) a polypeptide with at least 95% identity with the amino acid sequence from amino acid residues 21 to 259 of SEQ ID NO:14;
k) a polypeptide comprising an amino acid sequence from amino acid residues 20 to 296 of SEQ ID NO:16;
l) a polypeptide with at least 80% identity with the amino acid sequence from amino acid residues 20 to 296 of SEQ ID NO:16;
m) a polypeptide comprising an amino acid sequence from amino acid residues 20 to 248 of SEQ ID NO:17;
n) a polypeptide with at least 98% identity with the amino acid sequence from amino acid residues 20 to 248 of SEQ ID NO:17; and
o) a functional fragment of (a) to (n).

Preferably, the compositions are formulated with at least one carrier, preferably to provide desirable characteristics such as low color, low odor and acceptable storage stability.

The composition may comprise one of the above polypeptides as the major component, e.g., a mono-component composition. Alternatively, the composition may comprise one or more additional enzymes selected from the section “Detergency enzymes”. Preferably, the enzyme(s) is selected from the group consisting of proteases, cellulases, hemicellulases, lipases, cutinases, amylases, and pectinases, or mixtures thereof. More preferably, the enzymes are selected from the group consisting of metalloprotease, serine protease, triacylglycerol lipase, phospholipase A2, phospholipase A1, endoglucanses, xyloglucanases, alpha-amylases, laccases, pectate lyase, xylanases, and mannanases, or mixtures thereof.

The polypeptide compositions may be prepared in accordance with methods known in the art and may be in the form of a liquid, paste, gel or a dry formulation. For instance, the polypeptide may be formulated in the form of a granulate or a microgranulate. The polypeptide to be included in the composition may be stabilized in accordance with methods known in the art.

### Detergent Compositions

The present invention also encompass detergent composition comprising GH61 polypeptides, where the detergent composition may be adapted for specific uses such laundry, in particular household laundry, dish washing or hard surface cleaning.

One aspect of the invention is a detergent composition comprising at least one enzyme and at least one glycosyl hydrolase family 61 polypeptide, wherein the enzyme detergency benefit of said detergent is enhanced by at least 1 delta remission units as compared to a detergent without the glycosyl hydrolase family 61 polypeptide. Preferably the assessment is performed as described in the Materials and Method section using Laundrometer set-up A at a water hardness of 24° FH for stain removal benefits or using the Small scale anti-redeposition washing method for anti-redeposition benefits. The detergent may include one or more of the enzymes described in the section “Detergency enzymes”, in particular the enzymes may be selected from the group consisting of proteases, cellulases, hemicellulases, lipases, cutinases, amylases, and pectinases, or mixtures thereof.

In a preferred aspect the detergent composition comprises an enzyme is a stain removing enzyme selected from the group consisting of proteases, alpha-amylases, lipases and mannanases.

In a preferred embodiment the detergent composition comprises enzymes selected from the group consisting of metalloprotease, serine protease, triacylglycerol lipase, phospholipase A2, phospholipase A1, endoglucanses, xyloglucanases, alpha-amylases, pectate lyase, xylanases, and mannanases or mixtures thereof.

In another preferred aspect the detergent composition comprises one or more of the GH 61 polypeptides presented in Table 1, more preferred the detergent composition comprises one or more of the GH 61 polypeptides presented in Table 2, even more preferred the detergent composition comprises one or more of the GH 61 polypeptides presented in Table 3.

A detergent composition according to the present invention preferably comprises in the range of 0.00025 to 1.5% GH61 polypeptide by weight of the composition (w/w), more preferably in the range of 0.0005 to 1% GH61 polypeptide by weight of the composition (w/w) or in the range of 0.001 to 0.75 GH61 polypeptide by weight of the composition (w/w), even more preferably in the range of 0.0015 to 0.5 GH61polypeptide by weight of the composition (w/w) and most preferably in the range of 0.002 to 0.5 GH61 polypeptide by weight of the composition (w/w).

The detergent composition typically comprises conventional detergent ingredients such as surfactants, builders, bleaches, enzymes and other ingredients.

The detergent composition can be in any form, such as a solid, liquid, paste, gel or any combination thereof. The composition may be in the form of a tablet, bar or pouch, including multi-compartment pouches. The composition can be in the form of a powder, for example a free-flowing powder, such as an agglomerate, spray-dried powder, encapsulate, extrudate, needle, noodle, flake, or any combination thereof. However, the composition is preferably in the form of a liquid, preferably a liquid laundry detergent composition.

### Surfactant

Typically, the detergent composition comprises (by weight of the composition) one or more surfactants in the range of 0% to 50%, preferably from 2% to 40%, more preferably from 5% to 35%, more preferably from 7% to 30%, most preferably from 10% to 25%, even most preferably from 15% to 20%. In a preferred embodiment the detergent is a liquid or powder detergent comprising less than 40%, preferably less than 30%, more preferably less than 25%, even more preferably less than 20% by weight of surfactant. The composition may comprise from 1% to 15%, preferably from 2% to 12%, 3% to 10%, most preferably from 4% to 8%, even most preferably from 4% to 6% of one or more surfactants. Preferred surfactants are anionic surfactants, non-ionic surfactants, cationic surfactants, zwitterionic surfactants, amphoteric surfactants, and mixtures thereof. Preferably, the major part of the surfactant is anionic.

Suitable anionic surfactants are soaps and those containing sulfate or sulfonate groups. Surfactants of the sulfonate type that come into consideration are (C9-C13-alkyl)benzenesulfonates and olefinsulfonates, the latter being understood to be mixtures of alkenesulfonates and hydroxyalkanesulfonates and -disulfonates, as obtained, for example, by sulfonation of C12-C18 monoolefins having a terminally or internally located double bond. Also suitable are (C12-C18)alkanesulfonates and esters of alpha-sulfo fatty acids (ester sulfonates), for example the alpha-sulfonated methyl esters of hydrogenated coconut, palm kernel or tallow fatty acids a alpha-sulfocarboxylic acids resulting from saponification of MES may be used.

Further suitable anionic surfactants are sulfonated fatty acid glycerol esters comprising mono-, di- and tri-esters and mixtures thereof.

Alk(en)yl sulfates to which preference is given are the alkali metal salts and the sodium salts of sulfuric acid monoesters of C12-C18 fatty alcohols, for example from coconut fatty alcohol, tallow fatty alcohol, lauryl, myristyl, cetyl or stearyl alcohol, or of C10-C20 oxo alcohols and sulfuric acid monoesters of secondary alcohols having that chain length. From the point of view of washing technology, special preference is given to C12-C16 alkyl sulfates and C12-C15 alkyl sulfates and also to C14-C15 alkyl sulfates. Suitable anionic surfactants are also alkane-2,3-diylbis(sulfates) that are prepared, for example, in accordance with U.S. Pat. No. 3,234,258 or U.S. Pat. No. 5,075,041.

Also suitable are the sulfuric acid monoesters of straight-chain or branched C7-C21 alcohols ethoxylated with from 1 to 6 mole of ethylene oxide, such as 2-methyl-branched C9-C11 alcohols with, on average, 3.5 mole of ethylene oxide (EO) or C12-C18 fatty alcohols with from 1 to 4 EO. Because of their high foaming characteristics, they are normally used in washing and cleaning compositions only at relatively low levels, for example at levels of from 1% to 5% by weight.

Anionic surfactants may also include diesters, and/or salts of monoesters, of sulfosuccinic acid with C8-C18 fatty alcohol residues or mixtures thereof. Special preference is given to sulfosuccinates in which the fatty alcohol residues have a narrow chain length distribution. It is likewise also possible to use alk(en)yl sulfosuccinates having preferably from 8 to 18 C-atoms in the alk(en)yl chain, or salts thereof.

Further anionic surfactants that come into consideration are fatty acid derivatives of amino acids, for example of methyltaurine (taurides) and/or of methylglycine (sarcosides). Further anionic surfactants that come into consideration are soaps. Saturated fatty acid soaps such as the salts of lauric acid, myristic acid, palmitic acid, stearic acid, hydrogenated erucic acid and behenic acid and soap mixtures derived from natural fatty acids, for example coconut, palm kernel or tallow fatty acids. The anionic surfactants, including the soaps, may be present in the form of their sodium, potassium or ammonium salts and in the form of soluble salts of organic bases such as mono-, di- or triethanolamine. The anionic surfactants may be present in the form of their sodium or potassium salts.

In other embodiments the invention relates to a method, wherein the anionic surfactant is a linear alkylbenzenesulfonate; alpha-olefinsulfonate; alkyl sulfate (fatty alcohol sulfate); alcohol ethoxysulfate; secondary alkanesulfonate; alpha-sulfo fatty acid methyl ester; alkyl- or alkenylsuccinic acid; soap; or any combination thereof.

The detergent composition may comprise from 1% to 15%, preferably from 2% to 12%, 3% to 10%, most preferably from 4% to 8%, even most preferably from 4% to 6% of one or more anionic surfactants.

The detergent composition may also comprise from 1 wt % to 10 wt % of non-ionic surfactant, preferably from 2 wt % to 8 wt %, more preferably from 3 wt % to 7 wt %, even more preferably less than 5 wt % of non-ionic surfactant.

As non-ionic surfactants, preferably alkoxylated, advantageously ethoxylated and/or propoxylated, especially primary alcohols having from 8 to 18 C-atoms and, on average, from 1 to 12 moles of ethylene oxide (EO) and/or from 1 to 10 moles of propylene oxide (PO) per mole of alcohol are used. Special preference is given to C8-C16 alcohol alkoxylates, advantageously ethoxylated and/or propoxylated C10-C15 alcohol alkoxylates, especially C12-C14 alcohol alkoxylates, having a degree of ethoxylation between 2 and 10, or between 3 and 8, and/or a degree of propoxylation between 1 and 6, or between 1.5 and 5. The alcohol residue may be preferably linear or, especially in the 2-position, methyl-branched, or may comprise a mixture of linear and methyl-branched chains, as are usually present in oxo alcohols. Special preference is given, however, to alcohol ethoxylates derived from linear alcohols of natural origin that contain from 12 to 18 C-atoms, for example coconut, palm and tallow fatty alcohol or oleyl alcohol, and on average from 2 to 8 EO per mole of alcohol. The ethoxylated alcohols include, for example, C12-C14 alcohols with 3 EO or 4 EO, C9-C11 alcohols with 7 EO, C13-C15 alcohols with 3 EO, EO, 7 EO or 8 EO, C12-18 alcohols with 3 EO, 5 EO or 7 EO, mixtures thereof, such as mixtures of C12-C14 alcohol with 3 EO and C12-C18 alcohol with 5 EO. The mentioned degrees of ethoxylation and propoxylation represent statistical averages which, for a specific product, can be a whole number or a fractional number. Preferred alcohol ethoxylates and propoxylates have a restricted homologue distribution (narrow range ethoxylates/propoxylates, NRE/NRP). In addition to those non-ionic surfactants, fatty alcohol ethoxylates having more than 12 EO may also be used. Examples thereof are tallow fatty alcohol ethoxylate with 14 EO, 25 EO, 30 EO or 40 EO.

Also suitable are alkoxylated amines, which are ethoxylated and/or propoxylated, especially primary and secondary amines having from 1 to 18 C-atoms per alkyl chain and, on average, from 1 to 12 moles of ethylene oxide (EO) and/or from 1 to 10 moles of propylene oxide (PO) per mole of amine.

In addition, as further non-ionic surfactants, there may also be used alkyl polyglycosides of the general formula R₁O(G)ₓ, wherein R₁ is a primary straight-chain or methyl-branched (especially methyl-branched in the 2-position) alkyl group having from 8 to 22, preferably from 12 to 18, C-atoms and the symbol ‘G’ indicates a glycose (monosaccharide) unit having 5 or 6 C-atoms; preferably G is glucose. The degree of oligomerisation x, which indicates the average number of glycose units, will generally lie between 1 and 10; x is preferably from 1.2 to 1.4.

A further class of used non-ionic surfactants, which are used either as sole non-ionic surfactant or in combination with other non-ionic surfactants, comprises alkoxylated, preferably ethoxylated or ethoxylated and propoxylated fatty acid alkyl esters, having from 1 to 4 C-atoms in the alkyl chain, especially fatty acid methyl esters, as described, for example, in JP58/217598.

Non-ionic surfactants of the amine oxide type, for example N-(coco alkyl)-N,N-dimethylamine oxide and N-(tallow-alkyl)-N,N-bis(2-hydroxyethyl)amine oxide, and of the fatty acid alkanolamide or ethoxylated fatty acid alkanolamide type may also be suitable.

In some embodiments the invention relates to a method, wherein the non-ionic surfactant is an alcohol ethoxylate; nonylphenol ethoxylate; alkylpolyglycoside; alkyldimethylamineoxide; ethoxylated fatty acid monoethanolamide; fatty acid monoethanolamide; fatty acid (polyhydroxyalkanol)amide; N-acyl-N-alkyl derivatives of glucosamine (“glucamides”); or any combination thereof.

The detergent composition may also comprise from 0 wt % to 10 wt % of cationic surfactant, preferably from 0.1 wt % to 8 wt %, more preferably from 0.5 wt % to 7 wt %, even more preferably less than 5 wt % of cationic surfactant.

Suitable cationic surfactants are well known in the art and may comprise alkyl quaternary ammonium compounds, and/or alkyl pyridinium compounds and/or alkyl quaternary phosphonium compounds and/or alkyl ternary sulphonium compounds. The composition preferably comprises surfactant in an amount to provide from 100 ppm to 5,000 ppm surfactant in the wash liquor during the laundering process. The composition upon contact with water typically forms a wash liquor comprising from 0.1 g/L to 10 g detergent /L of wash solution, preferably from 0.5 to 10 g/L, 0.25 to 9 g/L, 0.5 to 8 g/L, 0.75 to 7 g/L, or 1 to 6 g detergent composition /L of wash solution, more preferably from 1.25 to 5.5 g/L or 1.5 to 5 g detergent composition /L of wash solution, more preferably from 2 to 4 g detergent composition /L of wash solution, most preferably from 2.5 to 3 g detergent composition /L of wash solution. Many suitable surface active compounds are available and fully described in the literature, for example, in “Surface-Active Agents and Detergents”, Volumes I and 11, by Schwartz, Perry and Berch.

### Builders

The main role of builder is to sequester divalent metal ions (such as calcium and magnesium ions) from the wash solution that would otherwise interact negatively with the surfactant system. The strength of the complex formed between the builder and Ca⁺⁺ and/or Mg⁺⁺, expressed as the log K value (either given as the equilibrium or stability constant or as the conditional stability constant at a given pH), may be in the range 3-8, particularly 5-8. The stability constant may be measured at 25° C. and ionic strength 0.1M, and the conditional stability constant may be measured at the same conditions at pH 8.5 or 9. Builders are also effective at removing metal ions and inorganic soils from the fabric surface, leading to improved removal of particulate and beverage stains. Builders are also a source of alkalinity and buffer the pH of the wash water to a level of 9.5 to 11. The buffering capacity is also termed reserve alkalinity, and should preferably be greater than 4 (the number of equivalents of a strong acid required to change the pH of one litre of a buffer solution by one unit, keeping the total amount of the acid and the salt in the buffer constant).

The detergent compositions of the present invention may comprise one or more detergent builders or builder systems. Many suitable builder systems are described in the literature, for example in Powdered Detergents, Surfactant science series volume 71, Marcel Dekker, Inc. Builder may comprise from 0% to 65%, preferably from 5% to 55%, more preferably from 10% to 40%, most preferably from 15% to 35%, even more preferably from 20% to 30% builder by weight of the subject composition. The composition may comprise from 0% to 15%, preferably from 1% to 12%, 2% to 10%, most preferably from 3% to 8%, even most preferably from 4% to 6% of builder by weight of the subject composition.

The builder may contain an amino group and may be, e.g., amino carboxylate, aminopolycarboxylate or a phosphonate. It may be a monomeric molecule comprising one, two or three amino groups (typically secondary or tertiary amino groups), and it may contain two, three, four or five carboxyl groups. Examples of suitable builders are methyl glycine diacetic acid (MGDA), glutamic acid N,N-diacetic acid (N,N-dicarboxymethyl glutamic acid tetrasodium salt, GLDA), nitrilotriacetic acid (NTA), diethylene triamine pentaacetic acid (DTPA), ethylenediaminetetraacetic acid (EDTA), Ethylenediamine-N,N′-disuccinic acid (EDDS), N-(1,2-dicarboxyethyl)-D,L-aspartic acid (IDS) and N-(2-hydroxyethyl)iminodiacetic acid (EDG), and salts thereof.

The builder may be an environmentally friendly sequesterant, e.g. as described in WO09/102,854. Suitable environmentally friendly sequesterants include one or more of amino acid-based sequesterants, succinate-based sequesterants, citric acid and salts thereof.

Examples of suitable amino acid based compounds include MGDA (methyl-glycine-diacetic acid), and salts and derivatives thereof and GLDA (glutamic-N,N-diacetic acid) and salts and derivatives thereof. Other suitable builders are described in U.S. Pat. No. 6,426,229. Particular suitable builders include; for example, aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts or ammonium salts thereof. In one aspect, GLDA salts and derivatives thereof may be employed. In one aspect, the tetrasodium salt of GLDA may be employed.

Further examples of suitable builders include N-(hydroxyethyl)-ethylidenediaminetriacetate (HEDTA), diethanolglycine (DEG), 1-Hydroxy Ethylidene-1,1-Diphosphonic Acid (HEDP), Diethylenetriamine Penta (Methylene Phosphonic acid) (DTPMP), Ethylene diamine tetra(methylene phosphonic acid) (EDTMPA) and aminotris(methylenephosphonic acid) (ATMP).

Examples of suitable succinate compounds are described in U.S. Pat. No. 5,977,053. In one aspect, suitable succinate compounds include tetrasodium immino succinate.

Builders may be classified by the test described by M. K. Nagarajan et al., JAOCS, Vol. 61, no. 9 (September 1984), pp. 1475-1478 to determine the minimum builder level required to lower the water hardness at pH 10.5 from 200 ppm (as CaCO₃) to 10 ppm in a solution of a hypothetical detergent dosed at 0.200 percent, given as the weight percent builder in the hypothetical detergent. Alternatively, the determination may be made at pH 8.5 to reflect the lower pH of typical modern laundry detergents. Using this method at either pH, the required level may be 0-25% (strong), 25-35% (medium) or >35% (weak). More preferred are compositions including strong and medium builders, most preferred are compositions with strong builders.

The builder may be a strong builder such as methyl glycine diacetic acid (MGDA) or N,NDicarboxymethyl glutamic acid tetrasodium salt (GLDA); it may be a medium builder such as sodium tri-poly-phosphate (STPP), or it may be a weak builder such as sodium citrate. More preferred are compositions including strong and medium builders, most preferred are compositions with strong builders. Other examples of builders are zeolite, diphosphate, triphosphate, phosphonate, carbonate, nitrilotriacetic acid, ethylenediaminetetraacetic acid (EDTA), diethylenetriaminepentaacetic acid, alkyl- or alkenylsuccinic acid, soluble silicates and layered silicates (e.g. SKS-6 from Hoechst).

### Bleaches

The detergent compositions of the present invention may comprise one or more bleaching agents. In particular powdered detergents may comprise one or more bleaching agents. Suitable bleaching agents include other photobleaches, pre-formed peracids, sources of hydrogen peroxide, bleach activators, hydrogen peroxide, bleach catalysts and mixtures thereof. In general, when a bleaching agent is used, the compositions of the present invention may comprise from about 0.1% to about 50% or even from about 0.1% to about 25% bleaching agent by weight of the subject cleaning composition.

Examples of suitable bleaching agents include:

(1) photobleaches for example Vitamin K3 or sulfonated zinc phthalocyanine;

(2) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxone, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;

(3) Sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include inorganic salts such as alkali metal silicate, carbonate or borate salts or mixtures thereof, or organic materials such as water-soluble or dispersible polymers, waxes, oils or fatty soaps. Useful bleaching compositions are described in U.S. Pat. Nos. 5,576,282, and 6,306,812;

(4) Bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are alkanolates and phenolates and derivatives thereof, one particular example being 4-oxidobenzenesulfonate and benzoic acid and derivatives thereof—especially benzene sulphonate. Suitable bleach activators include 4-(dodecanoyloxy)benzenesulfonate (LOBS), 4-(decanoyloxy)benzenesulfonate, 4-(decanoyloxy)benzoate (DOBS),4-(3,5,5-trimethylhexanoyloxy)benzenesulfonate (ISONOBS), tetraacetylethylenediamine (TAED) and 4-(nonanoyloxy)benzenesulfonate (NOBS). Suitable bleach activators are also disclosed in WO98/17767. Suitable bleach activators are also disclosed in WO 98/17767. While any suitable bleach activator may be employed, in one aspect of the invention the subject cleaning composition may comprise NOBS, TAED or mixtures thereof; and

(5) bleach catalysts that are capable of accepting an oxygen atom from peroxyacid and transferring the oxygen atom to an oxidizable substrate are described in WO2008/007319 (hereby incorporated by reference). Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. The bleach catalyst will typically be comprised in the detergent composition at a level of from 0.0005% to 0.2%, from 0.001% to 0.1%, or even from 0.005% to 0.05% by weight.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

In some embodiments the bleach components or systems may be selected from a group consisting of: peroxide-based bleaching systems (“peroxygen” or “oxygen-based”) such as sodium perborate mono- or tetrahydrate (NaBO₃.H₂O or NaBO₃.4H₂O), or sodium percarbonate (2Na₂CO₃.3H₂O₂); bleach activators such as TAED, NOBS, ISONOBS, LOBS or DOBS, all mentioned above; free peracids such as 6-(phthaloylamino)percapronic acid or 6-(phthalimido)peroxyhexanoic acid (PAP); bleach catalysts such as a mononuclear Schiff-base manganese(III) complex sold under the name Tinocat; photobleaches which are aluminum and zinc complexes of sulfonated phthalocyanine; or any combination thereof.

In some embodiments the bleach component may be an organic catalyst selected from the group consisting of organic catalysts having the following formulae:

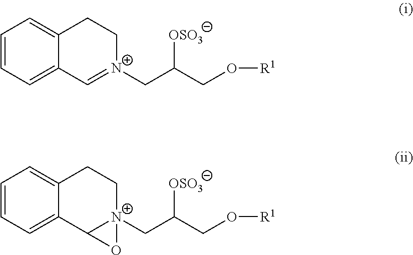

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl.

### Adjunct Materials

Dispersants—The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent—The detergent compositions of the present invention will preferably also contain additional components that may tint articles being cleaned, such as fluorescent whitening agent or optical brighteners. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of:
4,4′-bis-(2-diethanolamino-4-anilino-s-triazin-6-ylamino) stilbene-2,2′-disulphonate,
4,4′-bis-(2,4-dianilino-s-triazin-6-ylamino) stilbene-2,2′-disulphonate,
4,4′-bis-(2-anilino-4(N-methyl-N2-hydroxy-ethylamino)-s-triazin-6-ylamino) stilbene-2,2′-disulphonate,
4,4′-bis-(4-phenyl-2,1,3-triazol-2-yl)stilbene-2,2′-disulphonate,
4,4′-bis-(2-anilino-4(1-methyl-2-hydroxy-ethylamino)-s-triazin-6-ylamino) stilbene-2,2′-disulphonate and,
2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate.

Also preferred are fluorescent whitening agents is the commercially available Parawhite KX, supplied by Paramount Minerals and Chemicals, Mumbai, India.

Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Fabric hueing agents—The detergent compositions of the present invention may also include fabric hueing agents such as dyes or pigments which when formulated in detergent compositions can deposit onto a fabric when said fabric is contacted with a wash liquor comprising said detergent compositions thus altering the tint of said fabric through absorption of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch.

### Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs, as found for surfactants and lipids forming miceller, lamellar or other well defined meso-phases. Instead, many hydrotropes show a continuous-type aggregation process where the size of aggregates grows as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in laundry detergents may be utilized. Non-limiting examples of hydrotropes include sodium benzene sulfonate, sodium p-toluene sulfonates (STS), sodium xylene sulfonates (SXS), sodium cumene sulfonates (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Soil release polymers—The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxylethyl cellulose, hydroxylpropyl methyl cellulose, ester carboxy methyl cellulose, and mixtures thereof.

Anti-redeposition agents—The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, plant extracts, perfumes, pigments, sud suppressors, solvents, structurants for liquid detergents and/or structure elasticizing agents.

In one aspect the detergent is a compact fluid laundry detergent composition comprising: a) at least about 10%, preferably from 20 to 80% by weight of the composition, of surfactant selected from anionic surfactants, non ionic surfactants, soap and mixtures thereof; b) from about 1% to about 30%, preferably from 5 to 30%, by weight of the composition, of water; c) from about 1% to about 15%, preferably from 3 to 10% by weight of the composition, of non-aminofunctional solvent; and d) from about 5% to about 20%, by weight of the composition, of a performance additive selected from chelants, soil release polymers, enzymes and mixtures thereof; wherein the compact fluid laundry detergent composition comprises at least one of:

(i) the surfactant has a weight ratio of the anionic surfactant to the nonionic surfactant from about 1.5:1 to about 5:1, the surfactant comprises from about 15% to about 40%, by weight of the composition, of anionic surfactant and comprises from about 5% to about 40%, by weight of the composition, of the soap; (ii) from about 0.1% to about 10%, by weight of the composition, of a suds boosting agent selected from suds boosting polymers, cationic surfactants, zwitterionic surfactants, amine oxide surfactants, amphoteric surfactants, and mixtures thereof; and (ii) both (i) and (ii). All the ingredients are described in WO 2007/130562 hereby incorporated by reference in its entirety further polymers useful in detergent formulations are described in WO 2007/149806, which are hereby incorporated by reference in its entirety.

In another aspect the detergent is a compact granular (powdered) detergent comprising a) at least about 10%, preferably from 15 to 60% by weight of the composition, of surfactant selected from anionic surfactants, non ionic surfactants, soap and mixtures thereof; b) from about 10 to 80% by weight of the composition, of a builder, preferably from 20% to 60% where the builder may be a mixture of builders selected from i) phosphate builder, preferably less than 20%, more preferably less than 10% even more preferably less than 5% of the total builder is a phosphate builder; ii) a zeolite builder, preferably less than 20%, more preferably less than 10% even more preferably less than 5% of the total builder is a zeiolite builder; iii) citrate, preferably 0 to 5% of the total builder is a citrate builder; iv) polycarboxylate, preferably 0 to 5% of the total builder is a polycarboxylate builder v) carbonate, preferably 0 to 30% of the total builder is a carbonate builder and vi) sodium silicates, preferably 0 to 20% of the total builder is a sodium silicate builder; c) from about 0% to 25% by weight of the composition, of fillers such as sulphate salts, preferably from 1% to 15%, more preferably from 2% to 10%, more preferably from 3% to 5% by weight of the composition, of fillers; and d) from about 0.1% to 20% by weight of the composition, of enzymes, preferably from 1% to 15%, more preferably from 2% to 10% by weight of the composition, of enzymes.

In yet another aspect the detergent composition could also include an additive or a pre-spotter which is added to the wash to increase the general cleaning level, some of these additives may also be used as a pre-treatment agent applied to the textile before the washing step.

### EXAMPLES

### Materials and Methods

### Media

LB medium: per liter add 10 g of tryptone, 5 g of yeast extract, 5 g of sodium chloride

LB agar: add and 15 g of Bacto Agar to 1 L LB medium.

SOC media: Add the following to 900 ml of distilled H2O 20 g Bacto Tryptone, 5 g Bacto Yeast Extract, 2 ml of 5M NaCl, 2.5 ml of 1M KCl, 10 ml of 1M MgCl2, 10 ml of 1M MgSO4, 20 ml of 1M glucose. Adjust to 1 L with distilled H2O and sterilize by autoclaving.

1% TAE gel: 1% w/v agarose, 40 mM Tris-OH, 20 mM Acetic Acid, pH 7.8

### General GH61 Purification Protocol

Recombinant GH61 expressed in Aspergillus oryzae was identified by SDS-PAGE by comparing the background proteins from A. oryzae with the GH61 expression strain. In case of low expression and/or occurrence of host protein with similar expected molecular weight as the GH61 polypeptide, N-terminal sequencing was carried out on the purified protein to confirm the identity of the gene product. All chromatography runs were carried out in cold room at 4° C.

### Anion Exchange Chromatography

The conductivity of crude GH61 protein was lowered to less than 2 mS/cm by dilution with deionized water. The broth was adjusted to a pH at least 2.5 units above the calculated pl of the target protein using dilute NaOH. The binding buffers were prepared by choosing a buffer salt with buffering capacity at calculated target pl+2.5 pH units. Final buffer salt concentration was 20 mM. The Elution buffer was based on the binding buffer with an addition of 1 M NaCl. The column used in the anion exchange chromatography was Q Sepharose Fast Flow (GE Healthcare, Uppsala, Sweden). The broth was applied at constant flow rate recommended by the manufacturer. The unbound protein fraction was collected for further analysis. The protein loading step was followed by a washing step with at least two column volumes of binding buffer until the absorbance at 280 nm in the eluate was stable. Proteins absorbed to the column were eluted by a linear sodium chloride gradient from 0 to 1.0 M with duration of 12 column volumes. The fractionated protein including the unbound fraction was analyzed using SDS-PAGE. Fractions containing protein with the previously identified apparent molecular weight were pooled for further fractionation using hydrophobic interaction chromatography. Often, the GH61 protein was found in the unbound protein fraction.

### Hydrophobic Interaction Chromatography

The pool containing the GH61 polypeptide from the previous chromatography step was mixed with equal volume 3.2 M ammonium sulfate in 40 mM 3-[4-(2-hydroxyethyl)-1-piperazinyl] propanesulfonic acid (EPPS), pH 8.5. The mixture was examined by eye for protein precipitation and unless protein precipitation was observed, the GH61 containing solution was subjected to the chromatography step described below. If visible precipitation occurred, the ammonium sulphate concentration was lowered to 1.2 M by dilution with 20 mM EPPS, pH 8.5. The pool was then further fractionated using hydrophobic interaction chromatography (Phenyl Sepharose High Performance, GE Healthcare). The pool was applied on the column followed by at least two column volumes of binding buffer (20 mM EPPS+1.6 or 1.2M ammonium sulfate) to remove unbound protein. Elution was done by a linear ammonium sulfate gradient (1.6/1.2 to 0 M ammonium sulfate) for 12 column volumes. The fractionated peaks were evaluated for purity using SDS-PAGE and fractions containing the previously identified protein were pooled. The polypeptide concentration was determined by absorbance at 280 nm using molar extinction coefficient calculated using the software GPMAW (Lighthouse data, Odense, Denmark). The purified GH61 polypeptides were at least 95% pure judged by SDS-PAGE analysis.

Before testing the ability of a purified GH61 sample to enhance the detergency benefit of an enzyme or enzyme mixture it is recommended to include a buffer exchange step before adding the GH61 sample to the test system. The buffer exchange step can reduce e.g. ammonium sulfate or other components, which may affect the test results. Such buffer exchange can be done by dialysis, gel filtration or diawash. The GH61 of interest should be exchanged into a buffer or solvent system, which in it-self has little effect on the test result or with a known composition, such that the proper controls can be included in the tests. A suitable buffer system could e.g. be a buffer of sodium acetate (5 to 50 mM), glycine (5 to 50 mM), or hydrogen carbonate (5 to 50 mM), optionally including stabilizing components, such as divalent metal ions (such as calcium or magnesium ions) or other metal ions able to stabilize the GH61 protein in solution. The buffer may also contain e.g. a small amount of a surfactant, e.g. Brij-35 or Triton X-100, to reduce binding to containers and pipettes. One such buffer could e.g. include 25 mM sodium acetate, 0.5 mM CaCl₂, 0.01% (w/v) Triton X-100, pH 5.

Preparation of Water with Artificial Hardness

Water with artificial hardness was prepared by adding CaCl₂ and MgCl₂ to ultrapure (type I) water (e.g. Milli-Q water) or deionized water. Ultra pure water was used for small scale wash trials (mini-Terg-O-Tometer wash and laundrometer wash), whereas de-ionized water was used for full-scale wash.

A German degree of hardness (symbol ° dH) is defined as the equivalent of 10 mg CaO per liter, that is, 10/56.08 mmol=0.178 mmol hardness metal ions per liter, whereas a French degree of hardness (symbol FH) is defined as the equivalent of 100 g of calcium carbonate (CaCO₃) in 10 m3 of water=0.100 mmol hardness metal ions per liter. The conversion factor between German degree of hardness and French degree of hardness is 1.78×° dH=FH.

For Laundrometer (referred to as LOM) or mini-Terg-O-Tometer (referred to as mini-TOM) washes:

A stock solution of 6000° dH (2:1) was prepared by adding 105 g (=0.713 mol) CaCl₂.2H₂O and 72.6 g (=0.357 mol) MgCl₂.6H₂O per L of Milli-Q water.

A stock solution of 6000° dH (4:1) was prepared by adding 126 g (=0.856 mol) CaCl₂.2H₂O and 43.5 g (=0.214 mol) MgCl₂.6H₂O per L of Milli-Q water.

A water hardness of 13.45° dH (24 FH) was made by adding 4.48 mL of a 6000° dH (2:1) stock solution to Milli-Q water in a final volume of 2 L.

A water hardness of 26.9° dH (48 FH) was made by adding 8.97 mL of a 6000° dH (4:1) stock solution to Milli-Q water in a final volume of 2 L.

For full scale anti-redeposition washes:

A water hardness of 15° dH (26.7 FH) (4:1) was made by adding 472 g of CaCl₂.2H₂O and 163 g of MgCl₂.6H₂O to 1500 L of deionized water.

### List of Clean Textiles

TABLE 4

| Commercial | | |
|---|---|---|
| Textile Name | | |
| or number | Description | Manufacturer |
| Wfk80A | Knitted cotton, | wfk Testgewebe |
| | Area weight: 165 g/m², | GmbH, Germany |
| | Pick count: 175/170 pick/dm, | |
| | Weave: RRG, | |
| | Yarn count: 110 dtex. | |

### List of Stained Textiles

TABLE 5

| Stain | | | |
|---|---|---|---|
| number | Commercial | | |
| in the | Stain Name | | |
| examples | or Number | Manufacturers Description | Manufacturer |
| 1 | EMPA116 | Blood/milk/ink on cotton | EMPA Testmaterials AG, |
| | | | Switzerland |
| 2 | EMPA117 | Blood/milk/ink on polyester/cotton | EMPA Testmaterials AG, |
| | | | Switzerland |
| 3 | EMPA164 | Grass on cotton | EMPA Testmaterials AG, |
| | | | Switzerland |
| 4 | PC-05 | Blood, milk, ink on polyester/cotton (65/35), | Center For Testmaterials BV, |
| | | aged at elevated temperature | The Netherlands |
| 5 | PC-09 | Pigment, oil on polyester/cotton (65/35), | Center For Testmaterials BV, |
| | | aged at elevated temperature (<60 C.) | The Netherlands |
| 6 | PC-10 | Pigment oil/milk on polyester/cotton (65/35), | Center For Testmaterials BV, |
| | | aged at elevated temperature | The Netherlands |
| 7 | PC-S-27 | Potato starch, colored, on polyester/cotton (65/35) | Center For Testmaterials BV, |
| | | | The Netherlands |
| 8 | C-S-06 | Salad dressing with natural black, on cotton | Center For Testmaterials BV, |
| | | | The Netherlands |
| 9 | C-S-26 | Corn starch, colored, on cotton | Center For Testmaterials BV, |
| | | | The Netherlands |
| 10 | C-S-27 | Potato starch on cotton | Center For Testmaterials BV, |
| | | | The Netherlands |
| 11 | C-S-28 | Rice starch, colored, on cotton | Center For Testmaterials BV, |
| | | | The Netherlands |
| 12 | C-S-67 | Mustard on cotton | Center For Testmaterials BV, |
| | | | The Netherlands |
| 13 | C-S-73 | Locust bean gum, with pigment, on cotton | Center For Testmaterials BV, |
| | | | The Netherlands |
| 14 | Wfk10D | Sebum on cotton | wfk Testgewebe GmbH, Germany |
| 15 | PC-S-26 | Corn starch, colored, on polyester/cotton (65/35) | Center For Testmaterials BV, |
| | | | The Netherlands |
| 16 | PC-S-28 | Rice starch, colored, on polyester/cotton (65/35) | Center For Testmaterials BV, |
| | | | The Netherlands |
| 17 | PC-S-29 | Tapioca starch, colored, on polyester/cotton (65/35) | Center For Testmaterials BV, |
| | | | The Netherlands |
| 18 | P-S-26 | Corn starch, colored, on polyester | Center For Testmaterials BV, |
| | | | The Netherlands |
| 19 | P-S-27 | Potato starch, colored, on polyester | Center For Testmaterials BV, |
| | | | The Netherlands |
| 20 | P-S-28 | Rice starch, colored, on polyester | Center For Testmaterials BV, |
| | | | The Netherlands |
| 21 | C-S-29 | Tapioca starch, colored, on cotton | Center For Testmaterials BV, |
| | | | The Netherlands |
| 22 | 067KC | Ice Cream (chocolate) - Own Label on Cotton | Warwick Equest Ltd., UK |
| 23 | 004KC | Chocolate pudding on Cotton | Warwick Equest Ltd., UK |
| 24 | 049PE | Dirty motor oil on Polyester | Warwick Equest Ltd., UK |
| 25 | 062KC | Grass - Scrubbed on Cotton | Warwick Equest Ltd., UK |
| 26 | Rimmel Lipstick | Rimmel Lipstick Coral in Gold on Cotton | Warwick Equest Ltd., UK |
| | Coral in Gold KC | | |
| 27 | Black Tea KPE | Black Tea on Polyester | Warwick Equest Ltd., UK |
| 28 | PC-H080 | Grass/Mud on polyester/cotton (65/35) | Center For Testmaterials BV, |
| | | | The Netherlands |
| 29 | PC-H121 | Lipstick, Flametree on polyester/cotton (65/35) | Center For Testmaterials BV, |
| | | | The Netherlands |
| 30 | PC-H050 | Icecream, Chocolate Split on polycotton | Center For Testmaterials BV, |
| | | | The Netherlands |
| 31 | PC-H013 | Dirty motor oil on polyester/cotton (65/35) | Center For Testmaterials BV, |
| | | | The Netherlands |
| 32 | C-H033 | Choc. ice-cream with guar gum on Cotton | Center For Testmaterials BV, |
| | | | The Netherlands |
| 33 | C-H016 | Grass, with thixogum on Cotton | Center For Testmaterials BV, |
| | | | The Netherlands |
| 34 | C-H078 | Lipstick (lipase sensitive) on Cotton | Center For Testmaterials BV, |
| | | | The Netherlands |
| 35 | C-H028 | Tea on Cotton | Center For Testmaterials BV, |
| | | | The Netherlands |
| 36 | C-H039 | Grass, squeezed (no extract) on Cotton | Center For Testmaterials BV, |
| | | | The Netherlands |
| 37 | C-H065 | Sauce, green curry paste on Cotton | Center For Testmaterials BV, |
| | | | The Netherlands |

### Concentration of Enzymes Used in the Experiments/Examples

TABLE 6

| | | | | | | | Medium-high |
|---|---|---|---|---|---|---|---|
| | Low enzyme | | | | High enzyme | | enzyme conc. |
| | concentration | | | | concentration | | for full |
| | for LOM A wash | | | | for LOM B wash | | scale wash |
| | 24 FH | | 48 FH | | 24 or 48 FH | | 26.7 FH |
| Protease | 4.8 | nM | 24 | nM | 190 | nM | 51 nM |
| Amylase | 0.2 | nM | 0.47 | nM | 4.8 | nM | 3.0 nM |
| Lipase | 2.9 | nM | 2.9 | nM | 11 | nM | 6.8 nM |
| Mannanase | 0.059 | nM | 0.059 | nM | 1.5 | nM | 0.9 nM |

| | Enzyme concentration for small scale |
|---|---|
| | anti-redeposition wash (24 and 48 FH) |
| Cellulase | 0.005 nM |

The specific Enzyme products used were:

Savinase 16L, Novozymes A/S (contains protease of approximately 27 kDa)

Stainzyme 12L, Novozymes A/S (contains amylase of approximately 55 kDa)

Lipex 100L, Novozymes A/S (contains lipase of approximately 34 kDa)

Mannaway 25L, Novozymes A/S (contains mannanase of approximately 34 kDa)

Endolase 5000 L, Novozymes AS (contains cellulase of approximately 50 kDa)

### Prewashing of Textiles and Ballast

Textiles used in anti-redeposition washes and used for ballast in full scale washes were prewashed in two steps as described below.

In prewash step one the textile (3 kg) was washed in an EU household washing machine (Miele Softtronic W2245) in a normal wash at 60° C. using tap water (about 18° dH) and 5 g/L of IEC A* base detergent (Product order code: 88010-1, from Wfk Testgewebe GmbH, Christenfeld 10, D-41379 Brüggen, Germany) with added 1.1% by weight Celluclean 5.0T (Novozymes A/S) and 4% by weight Termamyl 60T (Novozymes A/S). After wash the textile was dried in a tumble dryer. In the second prewash step the textile was washed in an EU household washing machine in a normal wash at 95° C. using tap water (about 18° dH) and no detergent, after wash the textile was rinsed with deionized water using the rinse cycle on the washing machine. After wash the textile was dried in a tumble dryer.

After each use in a wash experiment the textile ballast was washed according to ballast prewash step two.

### Small Scale Anti-Redeposition Washing Method

Prewashed clean cotton fabric was washed in detergent containing carbon black (called “dirty detergent”). The washing was carried out in a mini-Terg-O-Tometer (called mini-TOM). During wash the carbon black soiling was deposited on the clean cotton fabric. After washing the cotton fabrics were rinsed and dried, and then measured with a spectrophotometer in order to detect the degree of soil redeposition in the form of carbon black.

Washing equipment: A mini-TOM consisting of 6 glass beakers placed in a temperature regulated water bath, with a rotating arm placed in each beaker for stirring which simulated the stirring in a Top-loader/Vertical drum washing machine. (The mini-TOM is a small-scale version of the Terg-O-Tometer test washing machine described in Jay C. Harris, “Detergency Evaluation and Testing”, Interscience Publishers LTD. (1954) pp. 60-61).

Beaker size: 250 mL

Washing solution volume: 100 mL of “dirty detergent” solution per mini-TOM beaker

Washing temperature: 30° C.

Washing time: 3 hours

Agitation: 150 rpm

Detergent: Liquid Persil Small and Mighty detergent base from Unilever (no enzymes), European detergent.

Detergent concentration: 1.23 g/L in water with hardness 24 or 48 FH.

Swatches/Cotton fabric: 8 pieces (5 cm×5 cm) wfk80A (see Table 4) prewashed as described above.

Cellulase and GH61 addition: Cellulase: 0 and 0.005 nM, GH61: 0 mg/L and 0.1 mg/L.

Carbon black concentration in wash: 5 mg/L

### Washing Procedure:

The “dirty detergent” solution was prepared by adding the detergent to water with artificial hardness. A carbon black stock solution was made by adding 100 mg carbon black (“carbon for detergency tests”, supplied by Sentakukagaku-kyokai, 2-11-1 Shimomaruko Ohta-ku, Tokyo 146-8620, Japan) to 50 mL detergent solution (2 mg/mL) and the solution was stirred for half an hour. The carbon black stock solution was then added to the detergent solution to a final concentration of 5 mg/L carbon black in the “dirty” detergent solution. The “dirty” detergent solution with carbon black was stirred until use. 100 mL of “dirty” detergent solution was put into each beaker; the beakers were placed in the mini-TOM, the water bath at 30° C. 8 pieces (5 cm×5 cm) of pre-washed wfk80A was placed in each beaker and stirring was switched on. Cellulase and or GH61 were then added to the beakers in different concentrations as described above.

When the enzyme and/or GH61 were added a timer was started. After 1 and 2 hours one textile swatch was removed from each beaker, rinsed in running tap water and spread out flat on filter paper. After 3 hours the remaining 6 swatches were removed from each beaker, rinsed in running tap water and left flat on filter paper and all the swatches were allowed to air dry at room temperature before remission measurement.

Remission measurements: Were made using a Macbeth 7000 Color Eye spectrophotometer. Each of the dry swatches was measured 4 times, 2 times on front and 2 times on back. As there is a risk of interference from the background, the swatches were placed on top of 2-6 layers of fabric during the measurement of the remission. The remission was measured at 500 nm. The UV filter was not included. An average result for remission for the swatches was then calculated. The anti-redeposition boosting effect of a given GH61 was calculated as the remission value of the swatches after wash in the presence of cellulase and GH61 minus the remission value of the swatch after wash with cellulase and no added GH61, i.e. the delta (Δ) remission; which reflects the enzyme anti-redeposition enhancing effect.

### Wash Tests for Evaluation of Stain Removal

When doing wash performance test for evaluation of stain removal in both full scale and in small scale the aim is often to make a simplified model of the conditions in a full scale wash by the end consumer. However, due to big variation in both type of textile as well as stain load and stain composition from wash load to wash load in a normal household it is impossible to cover all combinations or situations. Also when doing research and development it is important to control as many parameters as possible in order to be able to reproduce the results. To satisfy these concerns, model systems are used, where the type of stains added, the stain load, the type of fabric, and the amount of fabric is carefully controlled. Even in situations where the effect on one or a few particular stains is investigated it is recommended to include a series of other stains to best represent the situation in a normal household wash. As an example, it is recommended to include swatches with different soilings e.g. dirty motor oil, starch, milk or dairy products, grass, oil, blood, cocoa, tea, particulate soil or clay even when the focus of the study is e.g. removal of grass stains. It should also be noted that an improved stain removing effect on one stain may cause an apparently increased darkening of other types of stains or the fabric in general due to re-deposition. With some stains used in the wash tests there is a significant batch to batch variation of the stainload and/or coloration on the swatches before wash. To reduce this variation in the wash test it is recommended to cut these swatches into two and use one half for the reference wash (blank) and the other half in the wash with the GH61

### Laundrometer Method for Evaluation of Stain Removal

Pre-stained fabric swatches were washed in detergent with or without enzyme(s) and GH61 in a laundrometer (called LOM). After washing the swatches were rinsed and dried, and then measured with a spectrophotometer in order to detect the degree of stain removal.

Detergent: Liquid Persil Small & Mighty base detergent from Unilever (no enzymes), European detergent.

Detergent concentration: 1.23 g/l in water with hardness 24 or 48 FH.

Wash volume: 300 ml of detergent solution per LOM beaker.

Stained swatches: see Table 5 or 37, cut into 5×5 cm pieces

Washing equipment: LOM consisting of 20 stainless steel containers (500 mL) sealed with a rubber gasket and a lid placed in a temperature regulated water bath and fastened to a four sided stainless steel rotor which can hold five metal containers on each side (20 containers in total)

Rotor speed: 42 RPM

Mechanical agitation: 20 Stainless steel balls, 0.4 mm in diameter, were added to each container to give mechanical agitation during the wash.

Wash temperature: 40° C.

Enzyme and GH61 addition: Protease, amylase, lipase and/or mannanase in concentrations as seen in Table 6; GH61s: 0 mg/L, 0.1 and 0.5 mg/L.

Each condition was tested in two stainless steel containers.

### LOM Method A

The swatches were washed using the LOM method with enzyme(s) in low concentration as indicated in Table 6. The low enzyme concentration corresponds to a concentration on the steep part of the curve from a dose-response experiment at the relevant water hardness.

### LOM Method B

The swatches were washed using the LOM method with enzyme(s) in high concentration as indicated in Table 6. The high enzyme concentration corresponds to a concentration on the part of the curve where a plateau is reached from a dosis response experiment at the relevant water hardness.

### Washing Procedure:

The detergent solution was prepared by adding 1.23 g Persil Small&Mighty detergent pr L to water with artificial hardness. 300 mL of detergent solution was put into each container together with 20 steel balls. Maximum 11 pre-stained swatches (5×5 cm) were placed in each container. Enzyme(s) and (+/−) GH61 was then added to the containers in different concentrations as described above, and then each container was sealed by placing a rubber gasket in the lid and closing it. The closed containers were then placed in the laundrometer in the holes in the rotor and a metal restrainer bar was tightened over the container lids. The wash program (which simulates an EU washing machine wash cycle) was then started: the water bath was heated to 20° C., then 15 minutes wash where the temperature was gradually increased to 40° C. and finally 12 minutes wash while the temperature was held at 40° C. After this wash the swatches were removed from the containers, rinsed in tap water and left on filter paper for drying.

Remission measurements: Were made using a Macbeth 7000 Color Eye spectrophotometer. Each of the dry swatches was measured. As there is a risk of interference from the background, the swatches were placed on top of 4 layers of fabric during the measurement of the remission. The remission was measured at 460 nm. The UV filter was not included. An average result for remission for the swatches was calculated. The boosting of the stain removing enzymes of a given GH61 was calculated as the remission value of the swatches after wash in the presence of enzyme(s) and GH61 minus the remission value of the swatch after wash with enzyme(s) and no added GH61, i.e. the delta (Δ) remission; which reflects the enzyme detergency enhancing effect.

### Full Scale Washing Method for Stain Removal Experiments

Pre-stained fabric swatches were washed in detergent with or without enzyme(s) and GH61 in an EU household washing machine (Miele Softtronic W2245). After washing the swatches were dried, and then measured with a spectrophotometer in order to detect the degree of stain removal.

Detergent: European Liquid detergent Persil Small & Mighty base detergent from Unilever (no enzymes), or European liquid detergent Fairy Non Biological (no enzymes from Procter & Gamble).

Detergent concentration: 1.23 g/l or 3.69 g/L (respectively) in water with hardness 26.7 FH.

Stained swatches: see Table 5, number 1-21 cut into 5×5 cm pieces, number 22-37 are 5 cm circular stains on a 10×10 cm piece of textile, 2 or 3 of each in every wash

Wash temperature: 40° C., normal wash

Enzyme and GH61 addition: Protease, amylase, lipase and/or mannanase in concentrations as seen in Table 6; GH61s: 0 mg/L, 0.1 or 0.5 mg/L.

Ballast: up to 3 kg of mixed ballast textile (65/35% cotton/polyester) pr washing machine

### Washing Procedure:

The stained swatches were fastened to a larger piece of textile and placed in the washing machine together with the ballast, detergent and enzymes and/or GH61, and the machine was started. After wash the swatches were left on filter paper for drying.

Remission measurements: Were made using a Macbeth 7000 Color Eye spectrophotometer. Each of the dry swatches was measured. As there is a risk of interference from the background, the swatches were placed on top of 4 layers of fabric during the measurement of the remission. The remission was measured at 460 nm. The UV filter was not included. An average result for remission for the swatches was calculated. The boosting of the stain removing enzymes of a given GH61 was calculated as the remission value of the swatches after wash in the presence of enzyme(s) and GH61 minus the remission value of the swatch after wash with enzyme(s) and no added GH61, i.e. the delta (Δ) remission; which reflects the enzyme detergency enhancing effect.

### FTIR Analysis of Swatches After Wash

Fourier Transform Infrared Spectroscopy (FTIR) with the Bruker Vertex 70 with HTS-XT unit (Bruker Optik GmbH, Rudolf-Planck-Str. 27, 76275 Ettlingen, Germany) for diffuse reflection measurements was used as a measure of removal individual soil components from textiles.

Measurements were conducted directly on the textile swatches in the Mid Infrared Region (MIR) between 4000-400 cm-1 to monitor fundamental molecular vibrations (stretch and deformation) with high absorption coefficient at low sample concentration. The signal for the ester bond in fat has a maximum around 1720-1740 cm-1. The peak area between 1876 and 1681 cm-1 was used as a measure for the estimation of fat removal from the stained textile swatches containing fat. The signal for peptide bonds (amide I and II bands) has a maximum around 1650 cm-1 and 1550 cm-1, respectively, and the peak area from 1840 to 1430 cm-1 was used to calculate protein removal from the stained textile swatches containing protein. Multivariate analysis (Partial Component Analysis) (OPUS QUANT software; Bruker Optik GmbH, Rudolf-Planck-Str. 27, 76275 Ettlingen, Germany) was used to extract quantitative data. The peak areas of signals in the indicated zones of the unwashed swatches corrected for the signal of an unstained swatch of the same type, were set to 100% and the peak areas of the signals in the indicated zones of the washed swatches, corrected for the signal of an unstained swatch of the same type, were related to the unstained swatch.

### Example 1

### Cloning and Expression of Vt2 GH61 from Verticillium tenerum

The Verticillium tenerum, Vt2 GH61 polypeptide can be obtained in several ways.

For the present invention a cDNA library was made from Verticillium tenerum strain deposited at CBS as CBS109513, termed CBS109513 plasmid cDNA library. Generation of cDNA libraries is well known in the art and will not be described in further detail in the present invention but general methods for cDNA construction can be found in: Current Protocols in Molecular Biology 2007 by John Wiley and Sons, Inc.

The cDNA library was subjected to Transposon Assisted Signal Trapping (TAST) as described in patent WO 0177315-A1. Briefly, a plasmid cDNA library is treated with a transposon. The transposon contains a signal-less selectable marker at one of the transposon borders. In vitro insertion of the transposon into plasmids in the cDNA library occur essentially randomly. Trans-posons landing in frame and in the correct orientation and in an open reading frame encoding a secreted protein will result in a translational fusion consisting of the secretion signal and part of the target cDNA with the signal-less selectable marker. The transposon treated library is then transformed into E. coli and plated on the selective substance; in this case ampicillin. E. coli colonies growing under such selection usually contain a plasmid with an intron inserted into cDNA encoding a secreted or membrane bound protein. Picking many (hundreds to thousands) of these E. coli colony “trappants”, preparing plasmid from them and sequencing them with primers specific to the transposon or plasmid vector results in obtaining all or most of the sequence of those cDNAs. Use of standard bioinformatics techniques including Blast, one can identify secreted proteins in cDNA libraries. In this way the open reading frame of Vt2 GH61 was identified.

Based on the open reading frame two synthetic oligonucleotide primers, shown below, were designed to PCR amplify the full-length open reading frame from the Verticillium tenerum CBS109513 plasmid cDNA library generated above. The PCR primers were designed to amplify the entire open reading frames from the ATG start codon until the termination codon. The primers were synthesized with 15 base pair 5′ sequences homologous to the border of the cloning site for HindIII-BamHI cut pDau109 Aspergillus expression vector. pDau109 is disclosed in WO 2005042735, which is incorporated herein by reference.

(SEQ ID NO: 36)
F-Vt2 GH61 ACACAACTGGGGATCCACCATGAAGTACTCGCTCTCTCTA
(SEQ ID NO: 37)
R-Vt2 GH61 AGATCTCGAGAAGCTTAGACGTTGACCACAGCAGG

Bold letters represent coding sequence. The remaining sequence contains regions of homology to the pDau109 vector which make the resulting PCR fragment compatible with the INFUSION™ PCR Cloning Kit (Clontech Laboratories, Inc., Mountain View, Calif., USA). The underlined sequence contains the BamHI restriction site on the forward PCR primer (F-Vt2 GH61) and the HindIII restriction site on the reverse primer R-Vt2 GH61. Thus, the primers consisted of two regions, one region specific to the GH61 open reading frame and with an approximate annealing temperature of 50° C. or over, and the 15 base pairs homologous to the expression plasmid at the restriction enzyme borders.

Plasmid pDau109 was double digested with BamHI and HindIII and the vector was purified from the stuffer fragment by agarose gel electrophoresis and use of Illustra™ DNA and gel band purification kit (GE Healthcare).

The two primers were used in a PCR reaction to amplify a PCR fragment from the CBS109513 cDNA library. The cDNA library was diluted in TE buffer (10 mM Tris, 1 mM EDTA), pH 8.0 to 20 ng/μl. An MJ Research PTC-200 DNA engine was used to perform the PCR reaction. The following conditions were used:

| | | |
|---|---|---|
| 5X HF Pfusion buffer | 10 | μl |
| 10 μM dNTP | 0.5 | μl |
| 100 uM primer F | 1 | μl |
| 100 uM primer R | 1 | μl |
| CBS109513 cDNA library | 1 | μl |
| Deionized H₂O | 36 | μl |
| PFusion enzyme | 0.5 | μl |
| Total volume | 50 | μl |

The following PCR conditions were used:

98° C. for 30 seconds followed by 24 cycles of:

### 98° C. for 10 sec.

### 50° C. for 10 sec.

### 72° C. for 30 sec.

The reaction was then treated at 72° C. for 10 minutes and then the temperature reduced to 10C until the samples were recovered from the PCR cycler.

5 μl of the PCR sample was run on a 1% agarose TAE gel. Results showed that a single band of the predicted size (738 bp) was seen. The remaining PCR reaction was purified using Illustra DNA and gel band purification kit (GE Healthcare). The purified PCR product was then ready for cloning. The InFusion™ system for cloning was used for cloning the fragments into the prepared vector (BD Biosciences). The cloning protocol was followed exactly as described in the InFusion™ instruction manual generating a Vt2 GH61 construct. The treated plasmid and insert were transformed into InFusion™ Blue E. coli cells according to the protocol and plated on LB with 50 mg/liter ampicillin.

After incubating at 37° C. overnight, colonies were seen growing under selection on the LB ampicillin plates. 10 colonies transformed with the Vt2 GH61 construct were cultivated in LB liquid with 50 mg/ml ampicillin and plasmid was isolated according to the JETQUICK™ Plasmid Purification Spin Kit procedure (Genomed).

Isolated plasmids were sequenced with vector primers in order to determine a representative plasmid expression clone that was free of PCR errors. One error free Vt2 GH61 clone comprising Vt2 GH61 with SEQ ID NO: 22 was selected for further work. Plasmid DNA was isolated using the JETSTAR 2.0 Plasmid Mini/Midi/Maxi-Protocol (Genomed). The purified plasmid DNA was transformed into a standard fungal expression host, Aspergillus oryzae, according to the method of WO 2005/042735, pages 34-35, which are incorporated herein by reference. Aspergillus transformants able to produce the recombinant Vt2 GH61 polypeptide of SEQ ID NO: 12 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing Vt2 GH61 from Verticillium tenerum

Based on the nucleotide sequence identified as SEQ ID NO: 22, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector.

Using the two synthetic oligonucleotide primers F-Vt2 GH61 and R-Vt2 GH61 described above, a simple PCR reaction can be used to amplify the full-length open reading frame from the synthetic gene of SEQ ID NO: 22. The gene can then be cloned into an expression vector for example as described above and expressed in a host cell, for example in Aspergillus oryzae as described above. The GH61 polypeptide expressed in this way corresponds to SEQ ID NO: 12.

### Example 2

### Cloning and Expression of Vt1 GH61 from Verticillium tenerum

Primers were designed for a second GH61 identified in Verticillium tenerum as described in Example 1.

(SEQ ID NO: 38)
F-Vt1 ACACAACTGGGGATCCACCCATGAAGTTCACTGCCGTCT
(SEQ ID NO: 39)
R-Vt1 AGATCTCGAGAAGCTTAGCAGGTAATGGGACGGGG

Bold letters represent coding sequence. The remaining sequence contains regions of homology to the pDau109 vector which make the resulting PCR fragment compatible with the IN-FUSION PCR Cloning Kit (Clontech Laboratories, Inc., Mountain View, Calif., USA). The primers consisted of two regions, one region specific to the GH61 open reading frame and with an approximate annealing temperature of 50° C. or over, and the 15 base pairs homologous to the expression plasmid at the restriction enzyme borders.

A PCR reaction was performed as in Example 1 with the following exceptions:

Extensor Long PCR Master Mix, Buffer 1, ReddyMix™ version (AB Gene, cat. No. AB-0794) was used for the PCR amplification. The master mix contains buffer, dNTPs and a thermostable polymerase blend. The following concentrations were used:

| | | |
|---|---|---|
| PCR master mix: | 12.5 | μl |
| F-Vt1 (100 μM): | 0.5 | μl |
| R-Vt1 (100 μM): | 0.5 | μl |
| Deionized H2O | 10.5 | μl |
| CBS109513 cDNA library | 1 | μl |
| Total volume | 25 | μl |

The following PCR conditions were used:

### 95° C. 2 min

25 cycles of:

### 95° C. 15 sec.

### 50° C. 30 sec.

### 72° C. 60 sec.

Then 72° C. for 10 minutes. Samples were cooled to 10° C. before removal and further processing.

5 μl of PCR product were run on a 1% TAE Agarose gel. Results showed that a single band of the predicted size (742 bp) was seen. The remaining PCR reaction was purified using Illustra DNA and gel band purification kit (GE Healthcare). The purified PCR product was then ready for cloning. The InFusion™ system for cloning was used for cloning the fragments into the prepared vector (BD Biosciences). The cloning protocol was followed exactly as described in the InFusion™ instruction manual generating a Vt1 GH61 construct. The treated plasmid and insert were transformed into InFusion™ Blue E. coli cells according to the protocol and plated on LB with 50 mg/liter ampicillin.

After incubating at 37° C. overnight, colonies were seen growing under selection on the LB ampicillin plates. 10 colonies transformed with the Vt1 GH61 construct were cultivated in LB liquid with 50 mg/ml ampicillin and plasmid was isolated according to the JETQUICK™ Plasmid Purification Spin Kit procedure (Genomed).

Isolated plasmids were sequenced with vector primers in order to determine a representative plasmid expression clone that was free of PCR errors. One error free Vt1 GH61 clone comprising Vt1 GH61 with SEQ ID NO: 21 was selected for further work. Plasmid DNA was isolated using the JETSTAR 2.0 Plasmid Mini/Midi/Maxi-Protocol (Genomed). The purified plasmid DNA was transformed into a standard fungal expression host, Aspergillus oryzae, according to the method of WO 2005/042735, pages 34-35, which are incorporated herein by reference. Aspergillus transformants able to produce the recombinant Vt1 GH61 polypeptide of SEQ ID NO: 11 as judged by SDS PAGE analysis were then fermented in either small (200 ml) or very large (over 15 m³ tanks) to produce enough culture fluid for subsequent filtration, concentration and/or purification of the recombinant produced polypeptide. Purification was performed as described in the Materials and Methods section.

Alternative Method for Producing Vt1 GH61 from Verticillium tenerum,

Based on the nucleotide sequence identified as SEQ ID NO: 21, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector.

Using the two synthetic oligonucleotide primers F-Vt1 GH61 and R-Vt1 GH61 described above, a simple PCR reaction can be used to amplify the full-length open reading frame from the synthetic gene of SEQ ID NO: 21. The gene can then be cloned into an expression vector for example as described above and expressed in a host cell, for example in Aspergillus oryzae as described above. The GH61 polypeptide expressed in this way corresponds to SEQ ID NO: 11.

### Example 3

### Cloning and Expression of Pp 1 GH61 from Poronia punctata

The Poronia punctata, Pp1 GH61 polypeptide can be obtained in several ways.

For the present invention a cDNA library was made from a Poronia punctata strain. A similar strain is deposited at CBS as CBS 417.94. Generation of cDNA libraries is well known in the art and will not be described in further detail in the present invention but general methods for cDNA construction can be found in: Current Protocols in Molecular Biology 2007 by John Wiley and Sons, Inc.

The cDNA library was subjected to Transposon Assisted Signal Trapping (TAST) as described in patent WO0177315-A1, thereby isolating genes encoding secreted polypeptides. The isolated genes were subjected to sequencing and bioinformatics analysis. In this way the open reading frame of Pp1 GH61 was identified.

Primers were designed for the Pp1 GH61 as described in Example 1.

(SEQ ID NO: 40)
F-Pp1 ACACAACTGGGGATCCACCATGAAGACCTTTGCCCGCAT
(SEQ ID NO: 41)
R-Pp1 AGATCTCGAGAAGCTTAGCAGGACAGGGGAGCGG

A cDNA plasmid pool library for Poronia punctata was used as template for the PCR reaction. The sample was first diluted to 250 ng/μl with TE buffer (10 mM Tris, 1 mM EDTA), pH 8.0.

A PCR reaction was performed as in Example 2 with the following exceptions:

Extensor Long PCR Master Mix, Buffer 1, ReddyMix™ version (AB Gene, cat. No. AB-0794) was used for the PCR amplification. The master mix contains buffer, dNTPs and a thermostable polymerase blend. The following concentrations were used:

| | | |
|---|---|---|
| PCR master mix: | 25 | μl |
| F-Pp1 (100 μM): | 1 | μl |
| R-Pp1 (100 μM): | 1 | μl |
| Deionized H₂O | 22 | μl |
| P. punctata plasmid cDNA library 250 ng/μl | 1 | μl |
| Total volume | 50 | μl |

The following PCR conditions were used:

### 95° C. 30 sec

30 cycles of:

### 95° C. 5 sec.

### 50° C. 30 sec.

### 72° C. 60 sec.

Then 72° C. for 10 minutes. Samples were cooled to 10° C. before removal and further processing.

5 μl of PCR product were run on a 1% TAE Agarose gel. Results showed that a single band of the predicted size (800 bp) was seen. The remaining PCR reaction was purified using Illustra DNA and gel band purification kit (GE Healthcare). The purified PCR product was then ready for cloning. The InFusion™ system for cloning was used for cloning the fragments into the prepared vector (BD Biosciences). The cloning protocol was followed exactly as described in the InFusion™ instruction manual generating a Pp1 GH61 construct. The treated plasmid and insert were transformed into InFusion™ Blue E. coli cells according to the protocol and plated on LB with 50 mg/liter ampicillin.

After incubating at 37° C. overnight, colonies were seen growing under selection on the LB ampicillin plates. 2 colonies transformed with the Pp1 GH61 construct were cultivated in LB liquid with 50 mg/ml ampicillin and plasmid was isolated according to the JETQUICK™ Plasmid Purification Spin Kit procedure (Genomed).

Isolated plasmids were sequenced with vector primers in order to determine a representative plasmid expression clone that was free of PCR errors. One error free Pp1 GH61 clone comprising Pp1 GH61 with SEQ ID NO: 19 was selected for further work. Plasmid DNA is then isolated using the JETSTAR 2.0 Plasmid Mini/Midi/Maxi-Protocol (Genomed). The purified plasmid DNA was transformed into a standard fungal expression host, Aspergillus oryzae, according to the method of WO 2005/042735, pages 34-35, which are incorporated herein by reference. Aspergillus transformants able to produce the recombinant Pp1 GH61 polypeptide of SEQ ID NO: 9 as judged by SDS PAGE analysis were then fermented in either small (200 ml) or very large (over 15 m³ tanks) to produce enough culture fluid for subsequent filtration, concentration and/or purification of the recombinant produced polypeptide. Purification was performed as described in the Materials and Methods section.

Alternative Method for Producing Pp1 GH61 from Poronia Punctata

Based on the nucleotide sequence identified as SEQ ID NO: 19, a synthetic gene can be obtained from a number of vendors such as Gene Art (GENEART AG BioPark, Josef-Engert-Str. 11, 93053, egensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector.

Using the two synthetic oligonucleotide primers F-Pp1 GH61 and R-Pp1 GH61 described above, a simple PCR reaction can be used to amplify the full-length open reading frame from the synthetic gene of SEQ ID NO: 19. The gene can then be cloned into an expression vector for example as described above and expressed in a host cell, for example in Aspergillus oryzae as described above. The GH61 polypeptide expressed in this way corresponds to SEQ ID NO: 9.

### Example 4

### Cloning and Expression of Hi2 and Hi3 GH61 from Humicola insolens

The Humicola insolens, Hi2 and Hi3 GH61 polypeptides can be obtained in several ways.

For the present invention a cDNA library was made from a Humicola insolens strain as described in Example 1 of U.S. Pat. No. 5,457,046. The strain is deposited at DSMZ as DSM1800. Example 1 of U.S. Pat. No. 5,457,046 describes how the cDNA library was screened by hybridization using radioactive probes designed to anneal with the conserved part of the A-region of cellulase genes, corresponding to the carbohydrate binding module (CBM) of some cellulases. Furthermore, B-regions were identified. These consist of amino acid sequences that often link a CBM to the enzyme catalytic core. Example 1 of U.S. Pat. No. 5,457,046 discloses DNA sequences derived from the A-region amino acid sequences that are suitable for use as DNA probes in order to identify cDNAs encoding enzymes with CBMs. Using the methods described in Example 1 of U.S. Pat. No. 5,457,046 for cDNA library generation and cDNA library hybridization screening, two GH61 clones from Humicola insolens were identified.

### Cloning of Hi2 GH61

Primers were designed for the Hi2 GH61 as described in Example 1.

Hi2-RI
(SEQ ID NO: 42)
GCGGAATTCACCATGCACGTCCAGTCTCT
Hi2-NotI
(SEQ ID NO: 43)
ATTTGCGGCCGCACGCTTCGGCGTCCAGACCTTA

Bold letters represent Hi2 specific sequence. The underlined sequence contains the EcoRI restriction site on the forward PCR primer (Hi2-RI) and the NotI restriction site on the reverse primer Hi2-NotI. When the primers are used in a PCR reaction with cDNA from Humicola insolens, a fragment was produced that can be restricted with the enzymes EcoRI and NotI and thus produce a fragment that one can clone directionally into a suitable vector with the same restriction sites.

### The PCR Reaction:

Extensor Long PCR Master Mix, Buffer 1, ReddyMix™ version (AB Gene, cat. No. AB-0794) was used for the PCR amplification. The Plasmid cDNA library DSM1800 was diluted to 10 μg/μl in TE Buffer (10 mM Tris, 1 mM EDTA), pH 8.0 before the experiment. The master mix contains buffer, dNTPs and a thermostable polymerase blend. The following concentrations were used:

| | | |
|---|---|---|
| PCR master mix: | 12.5 | μl |
| Hi2-RI (100 μM) | 0.5 | μl |
| Hi2-NotI (100 μM): | 0.5 | μl |
| Deionized H₂O | 9.5 | μl |
| DSM1800 plasmid cDNA library 250 ng/μl | 2 | μl |
| Total volume | 25 | μl |

The following PCR conditions were used:

94° C. 2 minutes
10 cycles of:

### 94° C. 10 sec.

### 65° C. 30 sec.

68° C. 2 minutes

Then 20 cycles of:

### 94° C. 10 sec.

### 65° C. 30 sec.

68° C. 2 min+20 seconds/cycle

Then 68° C. for 7 minutes. The sample was then held at 10° C. until removed from the PCR machine.

5 μl of PCR product were run on a 1% TBE Agarose gel. Results showed that a single band of the predicted size (900 bp) was seen. The remaining PCR reaction was purified using Illustra DNA and gel band purification kit (GE Healthcare). The purified PCR product was then ready for cloning.

The methodology for cloning the Hi2 GH61 encoding sequence into a suitable expression vector and transformation of said vector into Aspergillus oryzae and selection of Aspergillus transformants producing Hi2 GH61 polypeptide is described in Example 2 of WO2005/080559. Briefly, the EcoRI-NotI restricted Hi2 PCR was ligated into EcoRI-NotI restricted pXYG1051 plasmid (WO2005/080559).

Ligation: pXYG1051 plasmid was diluted to 10 ng/μl in TE Buffer (10 mM Tris, 1 mM EDTA), pH 7.5

| | | |
|---|---|---|
| pXYG1051 (EcoRI-NotI digested) | 1 | μl |
| Hi2 PCR fragment (EcoRI-NotI digested) | 5 | μl |
| 10X T4 DNA ligase buffer (Promega) | 1 | μl |
| deionized water | 3 | μl |
| Total volume | 10 | μl |

0.2 μl of T4 DNA ligase was added to the reaction and the sample was incubated overnight at 16 degrees C. The sample was precipitated in ethanol:

| | |
|---|---|
| Sample | 10 μl |
| 3M Na acetate | 1 μl |
| 96% EtOH | 20 μl |

The sample was treated at −20° C. for 1 hour and then centrifuged at 10,000 g for 30 minutes. The pellet was aspirated and then washed twice with 70% EtOH by adding 25 μl 70% ethanol and centrifuging 10 minutes for each wash. The pellet was allowed to air dry before resuspension in 10 mM Tris pH 8.5.

Transformation into E. coli: 1 μl of the treated ligation was added to 40 μl of DH10B cells in a ice chilled BioRad 0.1 mm electroporation cuvette. The cuvette was placed in a BioRad Gene Pulser. The unit was set to 1.8 kV, 200 Ohms and 25 μF. After electroporation, the cuvette was filled with 1000 ml SOC media

and the suspended DH10B cells were then transferred to a Falcon 2059 tube. The tube was place in a rotary shaking incubator at 37° C. and 225 RPM for one hour. After the incubation, 10, 100 of the transformation were plated on LB agar with 100 mg/liter ampicillin. The plates were incubated overnight at 37° C. Twelve colonies were chosen from the several hundred that grew under selection and these were inoculated in 2 ml LB ampicillin (100 mg/l) in Falcon 2059 tubes. Plasmid DNA was isolated using the QiaPrep mini column purification procedure (Qiagen GmBH). The plasmid DNA was restricted with EcoRI and HindIII and the digests run on a 1% TBE gel. Electrophoresis results indicated that all 12 clones contained an insert of the correct size. Clones 2, 4, 6, 8 and 10 were therefore sequenced with an ABI 3730 XL Genetic analyzer. One error free Hi2 GH61 clone comprising Hi2 GH61 with SEQ ID NO: 20 was selected for all further work.

Clean plasmid of Hi2 GH61 used for Aspergillus transformation was produced from the E. coli cell line carrying this plasmid. Qiagen Midi ion exchange column was used for a 50 ml LB amp overnight culture of this strain. 3.5 μg of Hi2 GH61 was used to transform Aspergillus oryzae JAL355 (disclosed in WO 01/98484). Of the many Aspergillus colonies growing under selection, 22 were selected for further characterization. An Aspergillus transformant able to produce a recombinant Hi2 GH61 polypeptide of SEQ ID NO: 10 as judged by SDS PAGE analysis was then fermented in either small (200 ml) or very large (over 15 m³ tanks) to produce enough culture fluid for subsequent filtration, concentration and/or purification of the recombinant produced polypeptide. Purification was performed as described in the Materials and Methods section.

Alternative Method for Producing Hi2 GH61 from Humicola insolens

Based on the nucleotide sequence identified as SEQ ID NO: 20, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA. The synthetic gene can be designed to incorporate additional DNA sequences such as restriction sites or homologous recombination regions to facilitate cloning into an expression vector.

Using the two synthetic oligonucleotide primers F-Hi2 GH61 and R-Hi2 GH61 described above, a simple PCR reaction can be used to amplify the full-length open reading frame from the synthetic gene of SEQ ID NO: 20. The gene can then be cloned into an expression vector for example as described above and expressed in a host cell, for example in Aspergillus oryzae as described above. The GH61 polypeptide expressed in this way corresponds to SEQ ID NO: 10.

### Cloning of Hi3 GH61

Based on the nucleotide sequence identified as SEQ ID NO: 24, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA. The synthetic gene can be designed to incorporate additional DNA sequences such as restriction sites or homologous recombination regions to facilitate cloning into an expression vector.

Based on the sequence given as SEQ ID NO: 24, one can design infusion cloning primers:

(SEQ ID NO: 44)
F-Hi3: ACACAACTGGGGATCCACATGAAGGGACTTCTCAGCATCG
(SEQ ID NO: 45)
R-Hi3: AGATCTCGAGAAGCTTAGATGCACTGAGAGTAGTAAGCGTTCT

Using the two synthetic oligonucleotide primers F-Hi3 GH61 and R-Hi3 GH61 described above, a simple PCR reaction can be used to amplify the full-length open reading frame from the synthetic gene of SEQ ID NO: 24. The gene can then be cloned into an expression vector for example as described above and expressed in a host cell, for example in Aspergillus oryzae as described in example 1. The GH61 polypeptide expressed in this way corresponds to SEQ ID NO: 16.

### Example 5

### Cloning and Expression of Cg1 GH61 from Chaetomium globosum

The Cg1 GH61 polypeptide can be obtained in several ways. For identification of the Cg1 gene, the genome of Chaetomium globosum CBS148.51 has been subjected to genomic sequencing by the Broad Institute. The open reading frame of Cg1 GH61 was identified from the genome DNA sequence released by the Broad Institute. The genomic sequence to Cg1 was identified by performing a TFasty search against the nucleic acid sequences using several known GH61 protein sequences as queries. Tfasty compares a protein sequence to a DNA sequence database, calculating similarities with frameshifts to the forward and reverse orientations, and allowing frameshifts within codons. Tfasty is part of the FASTA3 program suite (Pearson, W.R. Flexible sequence similarity searching with the FASTA3 program package. Methods Mol. Biol. 2000; 132:185-219).

The genomic sequence which was identified is listed as SEQ ID NO: 26. The predicted, spliced cDNA, in which the introns have been removed is listed as SEQ ID NO: 23. Primers for Cg1 GH61 were designed as described in Example 1.

(SEQ ID NO: 46)
F-Cg1 ACACAACTGGGGATCCACCATGGCACCCTTGACATCCG
(SEQ ID NO: 47)
R-Cg1 AGATCTCGAGAAGCTTACGGGCCATCCCTGTTCG

Genome DNA from Chaetomium globosum CBS148.51 was isolated according to a modified FastDNA SPIN protocol. Briefly a FastDNA SPIN kit for soil, (Qbiogene cat. #6560-200) was used in an MP FastPrep-24 homogenization system (MP Biosciences cat. No. 6003500). Five ml of Chaetomium globosum was grown in 5 ml liquid culture for 48 h at 30° C. The medium used was YP medium with 2% glucose. Two ml fungal material from the cultures was harvested by centrifugation at 14000×g, 2 min. The supernatant was removed and the pellet resuspended in 500 μl H₂O. The suspension was transferred to a Lysing Matrix E FastPrep standard tube and 790 μl of sodium phosphate buffer and 100 μl MT buffer from the Fast DNA spin kit was added to the tube. The sample was then secured in the FastPrep Instrument and processed for 60 seconds at a speed of 5.5 m/sec. The sample was then centrifuged at 14000×g for two minutes and the supernatant transferred to a clean Eppendorf tube. 250 μl of PPS reagent from the Fast DNA spin kit was added and then the sample was mixed gently by inversion. The sample was again centrifuged at 14000×g for 5 minutes. The supernatant was transferred to a clean Falcon 2059 15 ml tube. 1 ml of Binding Matrix suspension was added and then mixed by inversion for two minutes. The sample was placed in a stationary tube rack and the silica matrix allowed to settle for 3 minutes. 500 μl of the supernatant was removed and discarded and then the remaining sample was resuspended in the matrix. This sample was then transferred to a SPIN filter tube and centrifuged at 14000×g for 1 minute. The catch tube was emptied and the remaining matrix suspension added to the SPIN filter tube. The sample was again centrifuged (14000×g, 1 min.). 500 μl of SEWS-M solution was added to the SPIN filter tube and the sample was centrifuged at the same speed for 1 minute. The catch tube was emptied and the SPIN filter replaced in the catch tube. The unit was centrifuged at 14000×g for 2 min. to “dry” the matrix of residual SEWS-M wash solution. The SPIN filter was placed in a fresh catch tube and allowed to air dry for 5 minutes at room temperature. The matrix was gently resuspended in 100 μl of DES (DNase/Pyrogen free water) with pipette tip. The unit was centrifuged (14000×g, 1 min.). The concentration of the DNA harvested from the catch tube was measured by a UV spectrophotometer at 260 nm.

The two primers were used in a PCR reaction to amplify a PCR fragment from the Chaetomium globosum CBS148.51 genomic DNA. The genomic DNA was diluted in TE Buffer (10 mM Tris, 1 mM EDTA), pH 8.0 to 100 ng/μl. An MJ Research PTC-200 DNA engine was used to perform the PCR reaction. The following conditions were used:

| | | |
|---|---|---|
| 5X HF Pfusion buffer | 10 | μl |
| 10 μM dNTP | 0.5 | μl |
| 100 mM primer F-Cg1 | 1 | μl |
| 100 mM primer R-Cg1 | 1 | μl |
| CBS148.51 genomic DNA | 1 | μl |
| deionized H₂O | 36 | μl |
| PFusion enzyme | 0.5 | μl |
| Total volume | 50 | μl |

The following PCR conditions were used:

98° C. for 30 seconds followed by 24 cycles of:

### 98° C. for 10 sec.

### 50° C. for 10 sec.

### 72° C. for 30 sec.

The reaction was then treated at 72° C. for 10 minutes and then the temperature reduced to 10° C. until the samples were recovered from the PCR cycler.

5 μl of the PCR sample was run on a 1% agarose TAE gel. Results showed that a single band of the predicted size (ca. 1000 bp.) was seen. The remaining PCR reaction was purified using Illustra DNA and gel band purification kit (GE Healthcare). The purified PCR product was then ready for cloning. The InFusion™ system for cloning was used for cloning the fragments into the prepared vector (BD Biosciences). The cloning protocol was followed exactly as described in the InFusion™ instruction manual generating a Cg1 GH61 construct. The treated plasmid and insert were transformed into InFusion™ Blue E. coli cells according to the protocol and plated on LB with 50 mg/liter ampicillin.

After incubating at 37° C. overnight, colonies were seen growing under selection on the LB ampicillin plates. 10 colonies transformed with the Cg1 GH61 construct were cultivated in LB liquid with 50 mg/ml ampicillin and plasmid was isolated according to the JETQUICK™ Plasmid Purification Spin Kit procedure (Genomed).

Isolated plasmids were sequenced with vector primers in order to determine a representative plasmid expression clone that was free of PCR errors. One error free Cg1 GH61 clone comprising Cg1 GH61 with SEQ ID NO: 26 was selected for further work. Plasmid DNA was isolated using the JETSTAR 2.0 Plasmid Mini/Midi/Maxi-Protocol (Genomed). The purified plasmid DNA was transformed into a standard fungal expression host, Aspergillus oryzae, according to the method of WO 2005/042735, pages 34-35, which are incorporated herein by reference. Aspergillus transformants able to produce the recombinant Cg1 GH61 protein of SEQ ID NO: 14 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing Cg1 GH61 from Chaetomiaum globosum,

Based on the nucleotide sequence identified as SEQ ID NO: 23, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA. The synthetic gene can be designed to incorporate additional DNA sequences such as restriction sites or homologous recombination regions to facilitate cloning into an expression vector.

Using the two synthetic oligonucleotide primers F-Cg1 GH61 and R-Cg1 GH61 described above, a simple PCR reaction can be used to amplify the full-length open reading frame from the synthetic gene of SEQ ID NO: 23. The gene can then be cloned into an expression vector for example as described above and expressed in a host cell, for example in Aspergillus oryzae as described above. The GH61 polypeptide expressed in this way corresponds to SEQ ID NO: 14.

### Example 6

### Cloning and Expression of At1 GH61 from Aspergillus terreus

The At1 GH61 polypeptide can be obtained in several ways. The genome of Aspergillus terreus NIH2624 has been subjected to whole genome shotgun sequencing by the Broad Institute of MIT and Harvard. The open reading frame of At1 GH61 was identified from the genome DNA sequence published by the Broad Institute using tfasty of the FASTA program package by WR Pearson (Pearson, W. R. (2000) Flexible sequence similarity searching with the FASTA3 program package Methods Mol. Biol. 132:185-219). The genomic sequence which was identified is publicly available as EMBL-EMI accession nr. AAJN01000191. The primers were synthesized based on this sequence with restriction enzyme recognition sites for cloning into a EcoRI-NotI cut pXYG1051 Aspergillus expression vector. pXYG1051 is disclosed in WO 2005/080559, which is incorporated herein by reference.

(SEQ ID NO: 48)
F-At1 TAAGAATTCACCATGCATTACCTGCACTCCGCT
(SEQ ID NO: 49)
R-At1 TATGCGGCCGCAGATACATGGCTCCTCAGGTAGA

Genome DNA from Aspergillus terreus ATCC28865 was isolated according to a modified FastDNA SPIN protocol as described in Example 5.

The two primers were used in a PCR reaction to amplify a PCR fragment from the Aspergillus terreus ATCC28865 genomic DNA. The genomic DNA was diluted in TE buffer (10 mM Tris pH 8.0, 1 mM EDTA) to 100 ng/μl. The PCR amplification reaction was composed of 1 μl of Aspergillus terreus ATCC28865 (100 ng/μl), 12.5 μl of 2×REDDYMIX™ PCR Buffer (THERMO Scientific), 1 μl of 5 μM primer F-At1, 1 μl of 5 μM primer R-At1, and 9.5 μl of H₂O. The amplification reaction was incubated in a MJ Research PTC-200 DNA ENGINE™ Thermal Cycler programmed for 1 cycle at 94° C. for 2 minutes; and 35 cycles each at 94° C. for 15 seconds and 60° C. for 1.5 minutes.

A 1.2 kb PCR reaction product was isolated by 1% agarose gel electrophoresis using TAE buffer and staining with SYBR® Safe DNA gel stain. The DNA band was visualized with the aid of an EAGLE EYE® Imaging System and a DARKREADER® Transilluminator. The 1.2 kb DNA band was excised from the gel and purified using a GFX® PCR DNA and Gel Band Purification Kit according to the manufacturer's instructions.

The 1.2 kb fragment was cleaved with Eco RI and Not I and purified using a GFX® PCR DNA and Gel Band Purification Kit according to the manufacturer's instructions.

The cleaved 1.2 kb fragment was then directionally cloned by ligation into Eco RI-Not I cleaved pXYG1051 (WO 2005/080559) using T4 ligase (Promega, Madison, Wis., USA) according to the manufacturer's instructions. The ligation mixture was transformed into E. coli TOP10F competent cells (Invitrogen Corp., Carlsbad, Calif., USA) according to the manufacturer's instructions. The transformation mixture was plated onto LB plates supplemented with 100 μg of ampicillin per ml. Plasmid minipreps were prepared according to the JETQUICK™ Plasmid Purification Spin Kit procedure (Genomed) from several transformants and sequenced. One plasmid with the correct At1 GH61 gene sequence was chosen (SEQ ID NO: 31). The predicted, spliced cDNA, in which the introns have been removed is listed as SEQ ID NO: 30. The expression vector pXYG1051 contains the same neutral amylase II (NA2) promoter derived from Aspergillus niger, and terminator elements as pCaHj483 (disclosed in Example 4 of WO 98/00529). Furthermore pXYG1051 has pUC18 derived sequences for selection and propagation in E. coli, and pDSY82 (disclosed in Example 4 of U.S. Pat. No. 5,958,727) derived sequences for selection and expression in Aspergillus facilitated by the pyrG gene of Aspergillus oryzae, which encodes orotidine decarboxylase and is used to complement a pyrG mutant Aspergillus strain.

The purified plasmid DNA was transformed into a standard fungal expression host, Aspergillus oryzae, according to the method of WO 2005/042735, pages 34-35, which are incorporated herein by reference. Aspergillus transformants able to produce the recombinant At1 GH61 polypeptide of SEQ ID NO: 3 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing At1 GH61 from Aspergillus terreus,

Based on the nucleotide sequence identified as SEQ ID NO: 30, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector for example as described above and expressed in a host cell, for example in Aspergillus oryzae as described above. The GH61 polypeptide expressed in this way corresponds to SEQ ID NO: 3.

### Example 7

### Cloning and Expression of At2 GH61 from Aspergillus terreus

The At2 GH61 polypeptide can be obtained in several ways. The genome of Aspergillus terreus NIH2624 has been subjected to whole genome shotgun sequencing by the Broad Institute of MIT and Harvard. The open reading frame of At2 GH61 was identified from the genome DNA sequence published by the Broad Institute using tfasty of the FASTA program package by WR Pearson (Pearson, W. R. (2000) Flexible sequence similarity searching with the FASTA3 program package Methods Mol. Biol. 132:185-219). The genomic sequence which was identified is listed as SEQ ID NO: 33 The predicted, spliced cDNA, in which the introns have been removed is listed as SEQ ID NO:32. The primers were synthesized with restriction enzyme recognition sites for cloning into a EcoRI-NotI cut pXYG1051 Aspergillus expression vector. pXYG1051 is disclosed in WO 2005/080559, which is incorporated herein by reference.

(SEQ ID NO: 50)
F-At2 TAAGAATTCATCATGAAGTACGCACTCGCT
(SEQ ID NO: 51)
R-At2 TATGCGGCCGCTTCCGCCTGTAGCAACCACT

Genome DNA from Aspergillus terreus ATCC28865 was isolated according to a modified FastDNA SPIN protocol as described in Example 5.

The two primers were used in a PCR reaction to amplify a PCR fragment from the Aspergillus terreus ATCC28865 genomic DNA as in Example 6.

A 0.8 kb PCR reaction product was isolated and cloned into pXYG1051 as described in Example 6.

One plasmid with the correct At2 GH61 gene sequence was chosen. The plasmid was designated pXYG1051-Q0CDX1.

The purified plasmid DNA was transformed into a standard fungal expression host, Aspergillus oryzae, according to the method of WO 2005/042735, pages 34-35, which are incorporated herein by reference. Aspergillus transformants able to produce the recombinant At2 GH61 polypeptide of SEQ ID NO: 13 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing At2 GH61 from Aspergillus terreus,

Based on the nucleotide sequence identified as SEQ ID NO: 32, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector for example as described above and expressed in a host cell, for example in Aspergillus oryzae as described above. The GH61 polypeptide expressed in this way corresponds to SEQ ID NO: 13.

### Example 8

### Cloning and Expression of At3 GH61 from Aspergillus terreus

The At3 GH61 polypeptide can be obtained in several ways. The genome of Aspergillus terreus NIH2624 has been subjected to whole genome shotgun sequencing by the Broad Institute of MIT and Harvard. The open reading frame of At3 GH61 was identified from the genome DNA sequence published by the Broad Institute using tfasty of the FASTA program package by W R Pearson (Pearson, W. R. (2000) Flexible sequence similarity searching with the FASTA3 program package Methods Mol. Biol. 132:185-219). A hypothetical frame shift error in the genomic sequence was identified by homology of the predicted 3′ end untranslated region with the query sequence (SEQ ID NO: 6). The genomic sequence which was identified is publicly available as EMBL-EMI accession nr. AAJN01000152. The primers were synthesized based on this sequence with restriction enzyme recognition sites for cloning into a EcoRI-NotI cut pXYG1051 Aspergillus expression vector. pXYG1051 is disclosed in WO 2005/080559, which is incorporated herein by reference.

(SEQ ID NO: 52)
F-At3 TAAGAATTCACAATGTCCCTGTCTAAGATTGCT
(SEQ ID NO: 53)
R-At3 TATGCGGCCGCAGGTGTTCGTAAGCCATGCT

Genome DNA from Aspergillus terreus ATCC28865 was isolated according to a modified FastDNA SPIN protocol as described in Example 5.

The two primers were used in a PCR reaction to amplify a PCR fragment from the Aspergillus terreus ATCC28865 genomic DNA as in Example 6.

A 0.8 kb PCR reaction product was isolated and cloned into pXYG1051 as described in Example 6.

One plasmid with the correct At3 GH61 gene sequence was chosen (SEQ ID NO: 29). The predicted, spliced cDNA, in which the introns have been removed is listed as SEQ ID NO: 25. The plasmid was designated pXYG1051-Q0CLL8

The purified plasmid DNA was transformed into a standard fungal expression host, Aspergillus oryzae, according to the method of WO 2005/042735, pages 34-35, which are incorporated herein by reference. Aspergillus transformants able to produce the recombinant At3 GH61 protein of SEQ ID NO: 17 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing At3 GH61 from Aspergillus terreus,

Based on the nucleotide sequence identified as SEQ ID NO: 25, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector for example as described above and expressed in a host cell, for example in Aspergillus oryzae as described above. The GH61 polypeptide expressed in this way corresponds to SEQ ID NO: 17.

### Example 9

### Cloning and Expression of At4 GH61 from Aspergillus terreus

The At4 GH61 polypeptide can be obtained in several ways. The genome of Aspergillus terreus NIH2624 has been subjected to whole genome shotgun sequencing by the Broad Institute of MIT and Harvard. The open reading frame of At4 GH61 was identified from the genome DNA sequence published by the Broad Institute using tfasty of the FASTA program package by W R Pearson (Pearson, W. R. (2000) Flexible sequence similarity searching with the FASTA3 program package Methods Mol. Biol. 132:185-219). The genomic sequence which was identified is listed as SEQ ID NO: 35. The primers were synthesized based on this sequence with restriction enzyme recognition sites for cloning into a EcoRI-NotI cut pXYG1051 Aspergillus expression vector. pXYG1051 is disclosed in WO 2005/080559, which is incorporated herein by reference.

(SEQ ID NO: 54)
F-At4 TAAGAATTCACCATGAAGTACCTTCCCACTCTTTTC
(SEQ ID NO: 55)
R-At4 TATGCGGCCGCAGACGCAGTGGACCGT

Genome DNA from Aspergillus terreus ATCC28865 was isolated according to a modified FastDNA SPIN protocol as described in Example 5.

The two primers were used in a PCR reaction to amplify a PCR fragment from the Aspergillus terreus ATCC28865 genomic DNA as in Example 6.

A 1.0 kb PCR reaction product was isolated and cloned into pXYG1051 as described in Example 6.

One plasmid with the correct At4 GH61 gene sequence was chosen (SEQ ID NO: 35). The predicted, spliced cDNA, in which the introns have been removed is listed as SEQ ID NO: 34. The plasmid was designated pXYG1051-Q0C7Z0

The purified plasmid DNA was transformed into a standard fungal expression host, Aspergillus oryzae, according to the method of WO 2005/042735, pages 34-35, which are incorporated herein by reference. Aspergillus transformants able to produce the recombinant At4 GH61 protein of SEQ ID NO: 18 as judged by SDS PAGE analysis were then fermented in either small (200 ml) or very large (over 15 m³ tanks) to produce enough culture fluid for subsequent purification of the recombinant produced polypeptide. Purification was performed as described in the Materials and Method section.

Alternative Method for Producing At4 GH61 from Aspergillus terreus,

Based on the nucleotide sequence identified as SEQ ID NO: 34, a synthetic gene can be obtained from a number of vendors such as Gene Art (GENEART AG BioPark, Josef-Engert-Str. 11, 93053, Regensburg, Germany) or DNA 2.0 (DNA2.0, 1430 O'Brien Drive, Suite E, Menlo Park, Calif. 94025, USA). The synthetic gene can be designed to incorporate additional DNA sequences such as restriction sites or homologous recombination regions to facilitate cloning into an expression vector for example as described above and expressed in a host cell, for example in Aspergillus oryzae as described above. The GH61 polypeptide expressed in this way corresponds to SEQ ID NO: 18.

### Example 10

### Cloning and Expression of Nc1 GH61 from Neurospora crassa

Neurospora crassa wild type strain FGSC2489 was used for isolation of genomic DNA. FGSC2489 is available from the Fungal Genetics Stock Center, School of Biological Sciences, Kansas City, Mo., USA. Genomic DNA can be isolated as described in Example 5.

Based on the genomic sequence given as SEQ ID NO: 27, one can design infusion cloning primers:

(SEQ ID NO: 56)
F-Nc1: ACACAACTGGGGATCCAACATGCGGTCCACTCTTGTCACC
(SEQ ID NO: 57)
R-Nc1: AGATCTCGAGAAGCTTAGACACACTGGGAGTAATAAGGAGGTG

Using the two synthetic oligonucleotide primers F-Nc1 GH61 and R-Nc1 GH61 described above, a simple PCR reaction can be used to amplify the full-length open reading frame from either genomic DNA of Neurospora crassa or from a synthetic gene of SEQ ID NO: 28. The gene can then be cloned into an expression vector for example as described above and expressed in a host cell, for example in Aspergillus oryzae as described in example 1. The GH61 polypeptide expressed in this way corresponds to SEQ ID NO: 4.

Nc1 (SEQ ID NO: 4) was purified by cellulose affinity chromatography essentially as described in “Enzymatic properties of cellulases from Humicola insolens.” Schülein, M., J Biotechnol (1997), vol 57(1-3):71-81.

### Example 11

### Cloning and Expression of Tt1 GH61 from Thielavia terrestris

The cloning and expression of Tt1 GH61 polypeptide (SEQ ID NO: 1) has been described in WO 2004/031378 Example 1, Example 4 and Example 5 hereby incorporated by reference. In WO 2004/031378 the sequence corresponding to Ta1 GH61 is given as SEQ ID NO: 2.

### Example 12

### Cloning and Expression of Hi1 GH61 from Humicola insolens

The cloning and expression of Hi1 GH61 polypeptide (SEQ ID NO: 5) has been described in WO 2004/031378 Example 2, Example 4 and Example 5 hereby incorporated by reference. In WO 2004/031378 the sequence corresponding to Ta1 GH61 is given as SEQ ID NO: 4 and the polynucleotide encoding the polypeptide sequence is given as SEQ ID NO: 3. Purification was performed as described in the Materials and Method section.

### Example 13

### Cloning and Expression of Tt2, Tt3, Tt4 and Tt5 GH61 from Thielavia terrestris

The cloning and expression of Tt2 GH61 polypeptide (SEQ ID NO: 2) has been described in WO 2005/074647 Example 8 to 14 hereby incorporated by reference. In WO 2005/074647 the sequence corresponding to Tt2 GH61 is named GH61G (SEQ ID NO: 10 in WO 2005/074647) and the polynucleotide encoding the polypeptide sequence is given as SEQ ID NO: 9 in WO 2005/074647 and is deposited in a plasmid is deposited with the accession number NRRL B-30811. Purification was performed as described in the Materials and Method section.

The cloning and expression of Tt3 GH61 polypeptide (SEQ ID NO: 6) has been described in WO 2005/074647 Example 1, 2 and 4 and Example 12 to 14 hereby incorporated by reference. In WO 2005/074647 the sequence corresponding to Tt3 GH61 is named GH61B (SEQ ID NO: 2 in WO 2005/074647) and the polynucleotide encoding the polypeptide sequence is given as SEQ ID NO: 1 in WO 2005/074647 and is deposited in a plasmid is deposited with the accession number NRRL B-30699. Tt3 (SEQ ID NO:6) was purified by cellulose affinity chromatography essentially as described in “Enzymatic properties of cellulases from Humicola insolens.” Schülein, M., J Biotechnol (1997), vol 57(1-3):71-81.

The cloning and expression of Tt4 GH61 polypeptide (SEQ ID NO: 7) has been described in WO 2005/074647 Example 1, 2 and 5 and Example 12 to 14 hereby incorporated by reference. hereby incorporated by reference. In WO 2005/074647 the sequence corresponding to Tt4 GH61 is named GH61 C (SEQ ID NO: 4 in WO 2005/074647) and the polynucleotide encoding the polypeptide sequence is given as SEQ ID NO: 3 in WO 2005/074647 and is deposited in a plasmid is deposited with the accession number NRRL B-30813. Purification was performed as described in the Materials and Method section.

The cloning and expression of Tt5 GH61 polypeptide (SEQ ID NO: 8) has been described in WO 2005/074647 Example 8 to 14 hereby incorporated by reference. In WO 2005/074647 the sequence corresponding to Tt5 GH61 is named GH61E (SEQ ID NO: 8 in WO 2005/074647) and the polynucleotide encoding the polypeptide sequence is given as SEQ ID NO: 7 in WO 2005/074647 and is deposited in a plasmid with the accession number NRRL B-30814. Purification was performed as described in the Materials and Method section.

### Example 14

### Cloning and Expression of Ta1 GH61 from Thermoascus aurantiaticus

The cloning and expression of Ta1 GH61 polypeptide (SEQ ID NO: 15) has been described in WO 2005/074656 Example 1 to 9 hereby incorporated by reference. In WO 2005/074656 the sequence corresponding to Ta1 GH61 is named T. aurantiaticus GH61A (SEQ ID NO: 2 in WO 2005/074656) and the polynucleotide encoding the sequence given as SEQ ID NO: 1 in WO 2005/074656 and is deposited in a plasmid with the accession number NRRL B-30704. Purification was performed as described in the Materials and Method section.

### Example 15

### Tt1 in Combination with Individual Stain Removing Enzymes in LOM

This example sets out to test the effect of Tt1 (0.1 mg/L or 0 mg/L) on stain removal from different pre-stained swatches. Tt1 was used in combination with a high concentration of the stain removing enzyme (see Table 6) in the LOM method B as described in the Material and Methods section and with a water hardness of 24 FH and the stains number 2-6, 8-9 and 11-14 as described in Table 5. The results are given as the remission value of the swatches after wash in the presence of enzyme and 0.1 mg/L Tt1 minus the remission value of the swatches after wash with enzyme and no added Tt1, i.e. the delta (Δ) remission; which reflects the enzyme detergency enhancing effect. The results are shown in Table 7.

TABLE 7

| Enzyme | Stain | | | |
|---|---|---|---|---|
| Protease (190 nM) | EMPA117 | PC-05 | CS-06 | CS-73 |
| | Blood/milk/ink | Blood/milk/ink | Salad dressing | Locust bean |
| | on polyester- | on polyester- | on cotton | gum on cotton |
| | cotton | cotton | | |
| | Δ2.0 | Δ2.8 | Δ1.0 | Δ2.8 |

| Amylase (4.8 nM) | | CS-26 | CS-28 |
|---|---|---|---|
| | | Corn starch | Rice starch |
| | | on cotton | on cotton |
| | | Δ3.0 | Δ2.7 |
| Lipase (11 nM) | CS-67 | CS-06 | CS-73 |
| | Mustard | Salad dressing | Locust bean |
| | on cotton | on cotton | gum on cotton |
| | Δ1.5 | Δ1.8 | Δ2.9 |

| Mannanase (1.5 nM) | PC-05 | CS-73 |
|---|---|---|
| | Blood/milk/ink | Locust bean |
| | on polyester- | gum on cotton |
| | cotton | |
| | Δ4.3 | Δ4.3 |

The results show that under different wash conditions and on different types of stains, here illustrated with protein (EMPA117 and PC-05), starch (CS-06, CS-26 and CS-28), fats and oil (CS-67) and mannan (CS-73 and CS-06), as well as different types of textiles, here illustrated with cotton and polyester/cotton, the combination of Tt1 together with a stain removing enzyme was capable of increasing the stain removing effect of the enzyme as compared to when the Tt1 is not present.

### Example 15a

### Tt1 in Combination with a Mixture of Stain Removing Enzymes in LOM

This example sets out to test the effect of Tt1 (0.1 mg/L or 0 mg/L) on stain removal from different pre-stained swatches. Tt1 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using stains number 1-6, 8, 11-14 as described in Table 5. The results are given as the remission value of the swatches after wash in the presence of enzymes and 0.1 mg/L Tt1 minus the remission value of the swatches after wash with enzymes and no added Tt1, i.e. the delta (Δ) remission; which reflects the enzyme detergency enhancing effect. The results are shown in Table 8.

TABLE 8

| Enzyme mixture | Stain | | |
|---|---|---|---|
| High concentration | Wfk10D | CS-73 | CS-28 |
| enzyme mixture | Sebum | Locust bean | Rice starch |
| Water hardness 24 FH | on cotton | gum on cotton | on cotton |
| | Δ1.4 | Δ2.8 | Δ2.0 |

| | | |
|---|---|---|
| Low concentration | CS-73 | CS-28 |
| enzyme mixture | Locust bean | Rice starch |
| Water hardness 24 FH | gum on cotton | on cotton |
| | Δ3.2 | Δ1.7 |

| | |
|---|---|
| Low concentration | CS-28 |
| enzyme mixture | Rice starch |
| Water hardness 48 FH | on cotton |
| | Δ4.4 |

The results show that under different wash conditions and on different types of stains, here illustrated with starch (CS-28), fats and oil (wfk10D) and mannan (CS-73), as well as different types of textiles, here illustrated with cotton, the combination of Tt1 together with a mixture of stain removing enzymes was capable of increasing the stain removing effect of the enzyme(s) as compared to when the Tt1 is not present.

### Example 15b

### Tt1 in Combination with a Mixture of Stain Removing Enzymes in Full Scale Wash

This example sets out to test the effect of Tt1 (0.1 mg/L, 0.5 mg/L or 0 mg/L) on stain removal from different pre-stained swatches. Tt1 was used in combination with a mixture of stain removing enzymes (protease, amylase, lipase, mannanase) in medium-high concentration as indicated in Table 6 in Material and Methods section. The tests were performed with the full scale set-up described in the Materials and Methods section with detergent Persil Small and Mighty, with a water hardness of 26.7 FH and using stains number 1-21 as described in Table 5. The results are given as the remission value of the swatches after wash in the presence of enzymes and Tt1 minus the remission value of the swatches after wash with enzymes and no added Tt1, i.e. the delta (Δ) remission; which reflects the enzyme detergency enhancing effect. The results are shown in Table 9.

TABLE 9

| Enzyme mixture | Stain | | | |
|---|---|---|---|---|
| Medium-high concentration enzyme mixture | | | EMPA164 | CS-73 |
| Tt1 (0.1 mg/L) | | | Grass | Locust bean |
| | | | on cotton | gum on cotton |
| | | | Δ1.4 | Δ2.3 |
| Medium- high | CS-26 | PC-05 | EMPA164 | PC-S-27 |
| concentration | Corn starch | Blood/milk/ink | Grass | Potato starch |
| enzyme mixture | on cotton | on polyester- | on cotton | on polyester- |
| Tt1 (0.5 mg/L) | | cotton | | cotton |
| | Δ1.8 | Δ1.4 | Δ1.7 | Δ1.6 |

The results show that on different types of stains, here illustrated with protein (EMPA164 and PC-05), starch (CS-26 and PC-S-27) and mannan (CS-73), as well as different types of textiles, here illustrated with cotton and polyester/cotton, the combination of Tt1 together with a mixture of stain removing enzymes was capable of increasing the stain removing effect of the enzyme(s) as compared to when the Tt1 is not present.

### Example 15c

### Example of Statistical Analysis of Wash Results with Tt1 in Combination with a Mixture of Stain Removing Enzymes in LOM

This example describes a statistical analysis of the wash results where the effect of adding GH61 to the wash liquor is compared to the results of a similar wash without addition of GH61. The conditions were as described in the Material and Methods section for LOM method B using Tt1 (0.5 mg/L or 0 mg/L) in combination with a high concentration of the mixture of stain removing enzymes (protease, amylase, lipase, mannanase). The stain set used was stain number 2-6, 8-9 and 11-14 as described in table 5 and the water hardness used was 24 FH.

The results are given as the remission value of the CS-28 and CS06 after wash in the presence of enzyme and 0.5 mg/L Tt1 (+GH61) or with 0 mg/L Tt1 (−GH61). The results are shown in Table 37.

TABLE 37

| | |
|---|---|
| CS-28 | CS06 |
| Remission | Remission |

| Run | −GH61 | +GH61 | −GH61 | +GH61 |
|---|---|---|---|---|
| 1 | 49.92 | 49.78 | 53.75 | 54.31 |
| 2 | 48.96 | 49.27 | 54.61 | 54.23 |
| 3 | 49.49 | 50.11 | 53.20 | 54.35 |
| 4 | 49.14 | 49.22 | 53.52 | 55.02 |
| 5 | 50.16 | 50.73 | 52.45 | 53.06 |
| 6 | 50.42 | 50.84 | 53.00 | 53.29 |
| 7 | 49.07 | 50.39 | 53.67 | 54.75 |
| 8 | 48.55 | 50.40 | 53.72 | 55.30 |
| 9 | 49.09 | 49.96 | 54.10 | 54.24 |
| 10 | 49.51 | 49.11 | 54.16 | 54.52 |
| 11 | 49.94 | 49.91 | 53.53 | 54.92 |
| 12 | 50.32 | 49.12 | 53.60 | 54.74 |
| 13 | 49.03 | 50.58 | 53.94 | 55.23 |
| 14 | 48.87 | 50.24 | 53.93 | 55.31 |
| 15 | 49.06 | 50.37 | 54.06 | 53.55 |
| 16 | 49.74 | 50.15 | 53.98 | 54.07 |
| 17 | 50.25 | 50.98 | 53.43 | 54.55 |
| 18 | 50.32 | 50.90 | 54.06 | 54.10 |
| 19 | 48.49 | 49.05 | 53.40 | 53.65 |
| 20 | 49.48 | 49.21 | 54.41 | 53.84 |
| Average | 49.491 | 50.016 | 53.726 | 54.351 |

The results show that under different wash conditions and on different types of stains, here illustrated with starch (CS-28), salad dressing (CS06), the combination of Tt1 together with a mixture of stain removing enzymes was capable of increasing the stain removing effect of the enzyme(s) as compared to when the Tt1 is not present.

To demonstrate the statistical significance of these values a Student's t-test analysis was performed on the values in Table 37.

Result of t-Test:

| | CS-28 | CS-06 |
|---|---|---|
| Delta value | 0.53 | 0.63 |
| Standard error of delta value | 0.20 | 0.18 |
| Degrees of freedom (df) | 38 | 38 |
| t Ratio (the value of the t-test) | 2.62 | 3.41 |
| t value (table)(5% two sided, df = 38)* | 2.025 | 2.025 |
| Probability > \|t\| (the p-value | 0.013 | 0.0015 |
| associated with the t-test) | | |

*from Abramowitz, Milton; Stegun, Irene A., eds. (1965), Handbook of Mathematical Functions with Formulas, Graphs, and Mathematical Tables, New York: Dover, pp. 948,

The found t-ratio of 2.62 (for CS-28) and 3.41 (CS-06) is clearly larger than the theoretical t-value of 2.025 (also known as the critical t-value) and the two means for the remission after wash with or without GH61 can thus be considered being significantly different at a significance level of 5%, demonstrating a statistically significant improved wash performance of adding GH61 to the wash. This is also seen from the very low p-value.

### Example 15d

### Tt1 in Combination with a Mixture of Stain Removing Enzymes in Full Scale Wash on Natural Stains

This example sets out to test the effect of Tt1 (0.5 mg/L or 0 mg/L) on stain removal from different pre-stained swatches. Tt1 was used in combination with a mixture of stain removing enzymes (protease, amylase, lipase, mannanase) in medium-high concentration as indicated in Table 6 in Material and Methods section. The tests were performed with the full scale wash set-up described in the Materials and Methods section with the detergent Fairy Non-biological with a water hardness of 26.7 FH and with a stain set corresponding to stain number 22-37 as described in Table 5. The results are given as the remission value of selected swatches after wash in the presence of enzymes and Tt1 minus the remission value of the swatches after wash with enzymes and no added Tt1, i.e. the delta (Δ) remission; which reflects the enzyme detergency enhancing effect. The results are shown in Table 38.

TABLE 38

| Enzyme mixture | Stain | | | |
|---|---|---|---|---|
| Medium- high | 062KC | PC-H080 | PC-H121 | C-H039 |
| concentration | Grass - | Grass/Mud on | Lipstick, Flame- | Grass, squeezed |
| enzyme mixture | Scrubbed | polyester/cotton | tree on polyester/ | (no extract) on |
| Tt1 (0.5 mg/L) | on Cotton | (65/35) | cotton (65/35) | Cotton |
| | Δ2.2 | Δ1.4 | Δ5.0 | Δ2.3 |

The results show that on different types of stains, here illustrated with protein (062KC, PC-H080 and C-H039), and fats and oils (PC-H121), as well as different types of textiles, here illustrated with cotton and polyester/cotton, the combination of Tt1 together with a mixture of stain removing enzymes was capable of increasing the stain removing effect of the enzyme(s) as compared to when the Tt1 is not present.

After the above full scale wash the following stains 062KC; PC-H121 and C-H039 were analyzed by FTIR as described in Materials and Methods section to determine the removal of individual soil components from the stains. Both unwashed stains and stains washed with and without Tt1 were analyzed. The results are shown in Table 39.

TABLE 39

| | | | % stain component | |
|---|---|---|---|---|
| | Stain | Unwashed | removal | |
| Stain | component | swatch | +Tt1 | −Tt1 |
| 062KC; Grass - | Protein | 100% | 87.6% | 90.6% |
| Scrubbed on Cotton | | | | |
| C-H039; Grass, | Protein | 100% | 93.1% | 95.9% |
| squeezed (no ex- | | | | |
| tract) on Cotton | | | | |
| PC-H121; Lipstick, | Fat | 100% | 54.4% | 60.6% |
| Flametree on | | | | |
| polester/cotton | | | | |

The data shows that stained textiles washed in the presence of GH61 has a reduced FTIR signal in the zones (frequencies) corresponding to the ester bond in fat and the amide bonds in proteins indicating that the presence of Tt1 has improved the removal of fat and protein from the stains.

### Example 16

### Ta1 in Combination with a Mixture of Stain Removing Enzymes in LOM

This example sets out to test the effect of Ta1 (0.1 mg/L or 0 mg/L) on stain removal from different pre-stained swatches. Ta1 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Ta1 minus the remission value of the swatches after wash with enzymes and no added Ta1, i.e. the delta (Δ) remission; which reflects the enzyme detergency enhancing effect. The results are shown in Table 10.

TABLE 10

| Enzyme mixture | Stain | |
|---|---|---|
| Low concentration enzyme mixture | EMPA116 | CS-28 |
| Water hardness 24 FH | Blood/milk/ink | Rice starch |
| | on cotton | on cotton |
| | Δ1.3 | Δ2.2 |

| Low concentration enzyme mixture | CS-28 |
|---|---|
| Water hardness 48 FH | Rice starch |
| | on cotton |
| | Δ1.5 |

The results show that under different wash conditions and on different types of stains, here illustrated with protein (EMPA116) and starch (CS-28), as well as different types of textiles, here illustrated with cotton, the combination of Ta1 together with a mixture of stain removing enzymes was capable of increasing the stain removing effect of the enzymes as compared to when the Ta1 is not present.

### Example 17

### Tt3 in Combination with a Mixture of Stain Removing Enzymes in LOM

This example sets out to test the effect of Tt3 (0.1 mg/L or 0 mg/L) on stain removal from different pre-stained swatches. Tt1 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Tt3 minus the remission value of the swatches after wash with enzymes and no added Tt3, i.e. the delta (Δ) remission; which reflects the enzyme detergency enhancing effect. The results are shown in Table 11.

TABLE 11

| Enzyme mixture | Stain | | |
|---|---|---|---|
| Low concentration | EMPA116 | CS-28 | PC-09 |
| enzyme mixture | Blood/milk/ink | Rice starch | Oil/pigment |
| Water hardness 24 FH | on cotton | on cotton | on polyester- |
| | | | cotton |
| | Δ1.1 | Δ1.6 | Δ1.1 |

The results show that under different wash conditions and on different types of stains, here illustrated with protein (EMPA116), starch (CS-28) and fats and oil (PC-09), as well as different types of textiles, here illustrated with cotton and polyester/cotton, the combination of Tt3 together with a mixture of stain removing enzymes was capable of increasing the stain removing effect of the enzymes as compared to when the Tt3 is not present.

### Example 18

### Nc1 in Combination with a Mixture of Stain Removing Enzymes in LOM

This example sets out to test the effect of Nc1 (0.1 mg/L or 0 mg/L) on stain removal from different pre-stained swatches. Nc1 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Nc1 minus the remission value of the swatches after wash with enzymes and no added Nc1, i.e. the delta (Δ) remission; which reflects the enzyme detergency enhancing effect. The results are shown in Table 12.

TABLE 12

| Enzyme mixture | Stain | | |
|---|---|---|---|
| High concentration | Wfk10D | CS-73 | CS-28 |
| enzyme mixture | Sebum | Locust bean | Rice starch |
| Water hardness 24 FH | on cotton | gum on cotton | on cotton |
| | Δ1.2 | Δ2.3 | Δ1.7 |

| | |
|---|---|
| Low concentration enzyme mixture | CS-28 |
| Water hardness 24 FH | Rice starch |
| | on cotton |
| | Δ2.5 |
| Low concentration enzyme mixture | CS-28 |
| Water hardness 48 FH | Rice starch |
| | on cotton |
| | Δ3.7 |

The results show that under different wash conditions and on different types of stains, here illustrated with starch (CS-28), fats and oil (wfk10D) and mannan (CS-73), as well as different types of textiles, here illustrated with cotton, the combination of Nc1 together with a mixture of stain removing enzymes was capable of increasing the stain removing effect of the enzymes as compared to when the Nc1 is not present.

### Example 18a

### Nc1 in Combination with a Mixture of Stain Removing Enzymes in Full Scale Wash

This example sets out to test the effect of Nc1 (0.1 mg/L or 0 mg/L) on stain removal from different pre-stained swatches. Nc1 was used in combination with a mixture of stain removing enzymes (protease, amylase, lipase, mannanase) in medium-high concentration as indicated in Table 6 in Material and Methods section. The tests were performed with the full scale set-up described in the Materials and Methods section with the detergent Persil Small and Mighty with a water hardness of 26.7 FH and using the stains number 1-21 as described in Table 5. The results are given as the remission value of the swatches after wash in the presence of enzymes and Nc1 minus the remission value of the swatches after wash with enzymes and no added Nc1, i.e. the delta (Δ) remission; which reflects the enzyme detergency enhancing effect. The results are shown in Table 13.

TABLE 13

| Enzyme mixture | Stain | | |
|---|---|---|---|
| Medium-high | CS-67 | CS-27 | PC-S-27 |
| concentration | Mustard | Potato starch | Potato starch |
| enzyme mixture | on cotton | on cotton | on polyester- |
| | | | cotton |
| | Δ1.9 | Δ2.2 | Δ1.5 |

The results show that on different types of stains, here illustrated with starch (CS-27 and PC-S-27) and fats and oil (CS-67), as well as different types of textiles, here illustrated with cotton and polyester/cotton, the combination of Nc1 together with a mixture of stain removing enzymes was capable of increasing the stain removing effect of the enzymes as compared to when the Nc1 is not present.

### Example 19

### Tt4 in Combination with a Mixture of Stain Removing Enzymes in LOM

This example sets out to test the effect of Tt4 (0.1 mg/L or 0 mg/L) on stain removal from different pre-stained swatches. Tt4 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Tt4 minus the remission value of the swatches after wash with enzymes and no added Tt4, i.e. the delta (Δ) remission; which reflects the enzyme detergency enhancing effect. The results are shown in Table 14.

TABLE 14

| Enzyme mixture | Stain | |
|---|---|---|
| Low concentration enzyme mixture | CS-73 | CS-28 |
| Water hardness 24 FH | Locust bean | Rice starch |
| | gum on cotton | on cotton |
| | Δ1.6 | Δ1.7 |

| | |
|---|---|
| Low concentration enzyme mixture | CS-28 |
| Water hardness 48 FH | Rice starch |
| | on cotton |
| | Δ3.0 |

The results show that under different wash conditions and on different types of stains, here illustrated with starch (CS-28) and mannan (CS-28), as well as different types of textiles, here illustrated with cotton, the combination of Tt4 together with a mixture of stain removing enzymes was capable of increasing the stain removing effect of the enzymes as compared to when the Tt4 is not present.

### Example 20

### Tt2 in Combination with Individual Stain Removing Enzymes

This example sets out to test the effect of Tt2 (0.1 mg/L or 0 mg/L) on stain removal from different pre-stained swatches. Tt2 was used in combination with a high concentration of the stain removing enzyme (see Table 6) in the LOM method B as described in the Material and Methods section with a water hardness of 24 FH and using the stains number 2-6, 8-9 and 11-14 as described in Table 5. The results are given as the remission value of the swatches after wash in the presence of enzyme and 0.1 mg/L Tt2 minus the remission value of the swatches after wash with enzyme and no added Tt2, i.e. the delta (Δ) remission; which reflects the enzyme detergency enhancing effect. The results are shown in Table 15.

TABLE 15

| Enzyme | Stain |
|---|---|
| Protease (190 nM) | PC-09 |
| | Oil/pigment on |
| | polyester-cotton |
| | Δ1.2 |
| Amylase (4.8 nM) | CS-28 |
| | Rice starch |
| | on cotton |
| | Δ1.7 |
| Lipase (11 nM) | CS-73 |
| | Locust bean |
| | gum on cotton |
| | Δ3.0 |
| Mannanase (1.5 nM) | CS-73 |
| | Locust bean |
| | gum on cotton |
| | Δ3.3 |

The results show that under different wash conditions and on different types of stains, here illustrated with starch (CS-28), fats and oil (PC-09) and mannan (CS-73), as well as different types of textiles, here illustrated with cotton and polyester/cotton, the combination of Tt2 together with a stain removing enzyme is capable of increasing the stain removing effect of the enzyme as compared to when the Tt2 is not present.

### Example 20a

### Tt2 in Combination with a Mixture of Stain Removing Enzymes in LOM

This example sets out to test the effect of Tt2 (0.1 mg/L or 0 mg/L) on stain removal from different pre-stained swatches. Tt2 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Tt2 minus the remission value of the swatches after wash with enzymes and no added Tt2, i.e. the delta (Δ) remission; which reflects the enzyme detergency enhancing effect. The results are shown in Table 16.

TABLE 16

| Enzyme mixture | Stain | | | |
|---|---|---|---|---|
| High concentration enzyme mixture | | Wfk10D | CS-73 | CS-28 |
| Water hardness 24 FH | | Sebum | Locust bean | Rice starch |
| | | on cotton | gum on cotton | on cotton |
| | | Δ1.2 | Δ1.2 | Δ1.2 |
| Low concentration enzyme mixture | EMPA116 | PC-09 | CS-73 | CS-28 |
| Water hardness 24 FH | Blood/milk/ink | Oil/pigment | Locust bean | Rice starch |
| | on cotton | on polyester- | gum on cotton | on cotton |
| | | cotton | | |
| | Δ1.4 | Δ1.0 | Δ2.6 | Δ2.6 |

| | |
|---|---|
| Low concentration enzyme mixture | CS-28 |
| Water hardness 48 FH | Rice starch |
| | on cotton |
| | Δ4.4 |

The results show that under different wash conditions and on different types of stains, here illustrated with protein (EMPA116), starch (CS-28), fats and oil (PC-09 and wfk10D) and mannan (CS-73), as well as different types of textiles, here illustrated with cotton and polyester/cotton, the combination of Tt2 together with a mixture of stain removing enzymes was capable of increasing the stain removing effect of the enzymes as compared to when the Tt2 is not present.

### Example 20b

### Tt2 in Combination with a Mixture of Stain Removing Enzymes in Full Scale Wash

This example sets out to test the effect of Tt2 (0.1 mg/L or 0 mg/L) on stain removal from different pre-stained swatches. Tt2 was used in combination with a mixture of stain removing enzymes (protease, amylase, lipase, mannanase) in medium-high concentration as indicated in Table 6 in Material and Methods section. The tests were performed with the full scale set-up described in the method section with the detergent Persil Small and Mighty, with a water hardness of 26.7 FH and using the stains number 1-21 as described in Table 5. The results are given as the remission value of the swatches after wash in the presence of enzymes and Tt2 minus the remission value of the swatches after wash with enzymes and no added Tt2, i.e. the delta (Δ) remission; which reflects the enzyme detergency enhancing effect. The results are shown in Table 17.

TABLE 17

| Enzyme mixture | Stain | | |
|---|---|---|---|
| Medium-high concentration | CS-26 | CS-27 | CS-73 |
| enzyme mixture | Corn starch | Potato starch | Locust bean |
| | on cotton | on cotton | gum on cotton |
| | Δ2.0 | Δ1.8 | Δ1.8 |

The results show that on different types of stains, here illustrated with starch (CS-26 and CS-27) and mannan (CS-73), as well as different types of textiles, here illustrated with cotton, the combination of Tt2 together with a mixture of stain removing enzymes was capable of increasing the stain removing effect of the enzymes as compared to when the Tt2 is not present.

### Example 21

### Tt5 in Combination with a Mixture of Stain Removing Enzymes in LOM

This example sets out to test the effect of Tt5 (0.1 mg/L or 0 mg/L) on stain removal from different pre-stained swatches. Tt5 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Tt5 minus the remission value of the swatches after wash with enzymes and no added Tt5, i.e. the delta (Δ) remission; which reflects the enzyme detergency enhancing effect. The results are shown in Table 18.

TABLE 18

| Enzyme mixture | Stain | |
|---|---|---|
| Low concentration enzyme mixture | CS-28 | PC-09 |
| Water hardness 24 FH | Rice starch | Oil/pigment on |
| | on cotton | polyester-cotton |
| | Δ2.0 | Δ1.1 |

The results show that under different wash conditions and on different types of stains, here illustrated with starch (CS-28) and fats and oil (PC-09), as well as different types of textiles, here illustrated with cotton and polyester/cotton, the combination of Tt5 together with a mixture of stain removing enzymes was capable of increasing the stain removing effect of the enzymes as compared to when the Tt5 is not present.

### Example 21a

### Tt5 in Combination with an Anti-Redeposition Enzyme in Mini-TOM

This example sets out to test the effect of Tt5 (0.1 mg/L) on anti-redeposition on cotton textile. Tt5 was used in combination with an anti-redeposition enzyme at 0.005 nM. The tests were performed with the mini-TOM set-up described in the Material and Methods section with the indicated water hardness. The results are given as the remission value of the swatches after wash in the presence of cellulase and 0.1 mg/L Tt5 minus the remission value of the swatches after wash with cellulase and no added Tt5, i.e. the delta (Δ) remission; which reflects the enzyme anti-redeposition enhancing effect. The results are shown in Table 19.

TABLE 19

| Enzyme | Result |
|---|---|
| Cellulase | Δ2.4 ± 0.6 |
| Water hardness 24 FH | |

The results show that the combination of Tt5 with an anti-redeposition enzyme was capable of increasing the anti-redeposition effect of the enzyme as compared to when the Tt5 is not present.

### Example 22

### Hi2 in Combination with a Mixture of Stain Removing Enzymes in LOM

This example sets out to test the effect of Hi2 (0.1 mg/L or 0 mg/L) on stain removal from different pre-stained swatches. Hi2 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Hi2 minus the remission value of the swatches after wash with enzymes and no added Hi2, i.e. the delta (Δ) remission; which reflects the enzyme detergency enhancing effect. The results are shown in Table 20.

TABLE 20

| Enzyme mixture | Stain | |
|---|---|---|
| High concentration enzyme mixture | CS-73 | CS-28 |
| Water hardness 24 FH | Locust bean | Rice starch |
| | gum on cotton | on cotton |
| | Δ2.3 | Δ1.2 |

| | |
|---|---|
| Low concentration enzyme mixture | CS-28 |
| Water hardness 24 FH | Rice starch |
| | on cotton |
| | Δ2.4 |
| Low concentration enzyme mixture | CS-28 |
| Water hardness 48 FH | Rice starch |
| | on cotton |
| | Δ5.2 |

The results show that under different wash conditions and on different types of stains, here illustrated with starch (CS-28) and mannan (CS-73), as well as different types of textiles, here illustrated with cotton, the combination of Hi2 together with a mixture of stain removing enzymes was capable of increasing the stain removing effect of the enzymes as compared to when the Hi2 is not present.

### Example 23

### At2 in Combination with a Mixture of Stain Removing Enzymes in LOM

This example sets out to test the effect of At2 (0.1 mg/L or 0 mg/L) on stain removal from different pre-stained swatches. At2 was used in combination with a mixture of stain removing enzymes (protease, amylase, lipase, mannanase). The tests were performed with a high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L At2 minus the remission value of the swatches after wash with enzymes and no added At2, i.e. the delta (Δ) remission; which reflects the enzyme detergency enhancing effect. The results are shown in Table 21.

TABLE 21

| | |
|---|---|
| Enzyme mixture | Stain |

| | | | |
|---|---|---|---|
| High concentration | EMPA164 | CS-28 | Wfk10D |
| enzyme mixture | Grass | Rice starch | Sebum |
| Water hardness 24 FH | on cotton | on cotton | on cotton |
| | Δ1.7 | Δ2.1 | Δ1.8 |

The results show that under different wash conditions and on different types of stains, here illustrated with protein (EMPA164), starch (CS-28) and fats and oil (wfk10D), as well as different types of textiles, here illustrated with cotton, the combination of At2 together with a mixture of stain removing enzymes was capable of increasing the stain removing effect of the enzymes as compared to when the At2 is not present.

### Example 23a

### At2 in Combination with an Anti-Redeposition Enzyme in Mini-TOM

This example sets out to test the effect of At2 (0.1 mg/L) on anti-redeposition on cotton textile. At2 was used in combination with an anti-redeposition enzyme at 0.005 nM. The tests were performed with the mini-TOM set-up described in the Material and Methods section with the indicated water hardness. The results are given as the remission value of the swatches after wash in the presence of cellulase and 0.1 mg/L At2 minus the remission value of the swatches after wash with cellulase and no added At2, i.e. the delta (Δ) remission; which reflects the enzyme anti-redeposition enhancing effect. The results are shown in Table 22.

TABLE 22

| Enzyme | Result |
|---|---|
| Cellulase | Δ5.7 ± 0.9 |
| Water hardness 24 FH | |
| Cellulase | Δ4.3 ± 0.8 |
| Water hardness 48 FH | |

The results show that the combination of At2 with an anti-redeposition enzyme was capable of increasing the anti-redeposition effect of the enzyme as compared to when the At2 is not present.

### Example 24

### At1 in Combination with Individual Stain Removing Enzymes in LOM

This example sets out to test the effect of At1 (0.1 mg/L or 0 mg/L) on stain removal from different pre-stained swatches. At1 was used in combination with a high concentration of the stain removing enzyme (see Table 6) in the LOM method B as described in the Material and Methods section with a water hardness of 24 FH and using the stains number 2-6, 8-9 and 11-14 as described in Table 5. The results are given as the remission value of the swatches after wash in the presence of enzyme and 0.1 mg/L At1 minus the remission value of the swatches after wash with enzyme and no added At1, i.e. the delta (Δ) remission; which reflects the enzyme detergency enhancing effect. The results are shown in Table 23.

TABLE 23

| Enzyme | Stain | | | |
|---|---|---|---|---|
| Protease | EMPA117 | PC-10 | PC-09 | CS-06 |
| (190 nM) | Blood/milk/ink | Oil/milk on | Oil/pigment | Salad |
| | on polyester- | polyester- | on polyester- | dressing on |
| | cotton | cotton | cotton | cotton |
| | Δ2.2 | Δ1.6 | Δ1.1 | Δ1.0 |

| Amylase (4.8 nM) | | PC-05 |
|---|---|---|
| | | Blood/milk/ink |
| | | on polyester- |
| | | cotton |
| | | Δ1.7 |
| Lipase (11 nM) | CS-06 | CS-73 |
| | Salad | Locust bean |
| | dressing | gum on |
| | on cotton | cotton |
| | Δ2.1 | Δ3.7 |
| Mannanase (1.5 nM) | PC-10 | CS-73 |
| | Oil/milk on | Locust bean |
| | polyester- | gum on |
| | cotton | cotton |
| | Δ1.3 | Δ6.3 |

The results show that under different wash conditions and on different types of stains, here illustrated with protein (EMPA117, PC-05 and PC-10), starch (CS-06), fats and oil (PC-09) and mannan (CS-73 and CS-06), as well as different types of textiles, here illustrated with cotton and polyester/cotton, the combination of At1 together with a stain removing enzyme is capable of increasing the stain removing effect of the enzyme as compared to when the At1 is not present.

### Example 24a

### At1 in Combination with a Mixture of Stain Removing Enzymes in LOM

This example sets out to test the effect of At1 (0.1 mg/L or 0 mg/L) on stain removal from different pre-stained swatches. At1 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L At1 minus the remission value of the swatches after wash with enzymes and no added At1, i.e. the delta (Δ) remission; which reflects the enzyme detergency enhancing effect. The results are shown in Table 24.

TABLE 24

| | |
|---|---|
| Enzyme mixture | Stain |

| | | |
|---|---|---|
| High concentration enzyme mixture | EMPA164 | CS-28 |
| Water hardness 24 FH | Grass | Rice starch |
| | on cotton | on cotton |
| | Δ1.4 | Δ2.2 |

| | |
|---|---|
| Low concentration enzyme mixture | CS-28 |
| Water hardness 48 FH | Rice starch |
| | on cotton |
| | Δ1.6 |

The results show that under different wash conditions and on different types of stains, here illustrated with protein (EMPA164) and starch (CS-28), as well as different types of textiles, here illustrated with cotton, the combination of At1 together with a mixture of stain removing enzymes was capable of increasing the stain removing effect of the enzymes as compared to when the At1 is not present.

### Example 24b

### At1 in Combination with a Mixture of Stain Removing Enzymes in Full Scale Wash

This example sets out to test the effect of At1 (0.1 mg/L or 0 mg/L) on stain removal from different pre-stained swatches. At1 was used in combination with a mixture of stain removing enzymes (protease, amylase, lipase, mannanase) in medium-high concentration as indicated in Table 6 in Material and Methods section and using the stains number 1-21 as described in Table 5. The tests were performed with the full scale set-up described in the Materials and Methods section and with the detergent Persil Small and Mighty, with a water hardness of 26.7 FH. The results are given as the remission value of the swatches after wash in the presence of enzymes and At1 minus the remission value of the swatches after wash with enzymes and no added At1, i.e. the delta (Δ) remission; which reflects the enzyme detergency enhancing effect. The results are shown in Table 25.

TABLE 25

| Enzyme mixture | Stain | | | | | | |
|---|---|---|---|---|---|---|---|
| Medium-high | EMPA164 | PC-05 | CS-26 | CS-27 | PC-S-27 | CS-67 | CS-73 |
| concentration | Grass | Blood/milk/ | Corn | Potato | Potato | Mustard | Locust |
| enzyme mixture | on cotton | ink on | starch | starch | starch on | on cotton | bean |
| | | polyester- | on cotton | on cotton | polyester- | | gum |
| | | cotton | | | cotton | | on cotton |
| | Δ2.3 | Δ1.0 | Δ1.0 | Δ2.4 | Δ1.4 | Δ1.0 | Δ2.2 |

The results show that on different types of stains, here illustrated with protein (EMPA164 and PC-05), starch (CS-26, CS-27 and PC-S-27), fats and oil (CS-67) and mannan (CS-73), as well as different types of textiles, here illustrated with cotton and polyester/cotton, the combination of At1 together with a mixture of stain removing enzymes was capable of increasing the stain removing effect of the enzymes as compared to when the Tt1 is not present.

### Example 24c

### At1 in Combination with an Anti-Redeposition Enzyme in Mini-TOM

This example sets out to test the effect of At1 (0.1 mg/L) on anti-redeposition on cotton textile. At1 was used in combination with an anti-redeposition enzyme at 0.005 nM. The tests were performed with the mini-TOM set-up described in the Material and Methods section with the indicated water hardness. The results are given as the remission value of the swatches after wash in the presence of cellulase and 0.1 mg/L At1 minus the remission value of the swatches after wash with cellulase and no added At1, i.e. the delta (Δ) remission; which reflects the enzyme anti-redeposition enhancing effect. The results are shown in Table 26.

TABLE 26

| Enzyme | Result |
|---|---|
| Cellulase | Δ10.3 ± 1.2 |
| Water hardness 24 FH | |
| Cellulase | Δ7.8 ± 1.0 |
| Water hardness 48 FH | |

The results show that the combination of At1 with an anti-redeposition enzyme was capable of increasing the anti-redeposition effect of the enzyme as compared to when the At1 is not present.

### Example 25

### Hi3 in Combination with a Mixture of Stain Removing Enzymes in LOM

This example sets out to test the effect of Hi3 (0.1 mg/L or 0 mg/L) on stain removal from different pre-stained swatches. Hi3 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Hi3 minus the remission value of the swatches after wash with enzymes and no added Hi3, i.e. the delta (Δ) remission; which reflects the enzyme detergency enhancing effect. The results are shown in Table 27.

TABLE 27

| | |
|---|---|
| Enzyme mixture | Stain |

| | | |
|---|---|---|
| High concentration enzyme mixture | EMPA164 | CS-28 |
| Water hardness 24 FH | Grass | Rice starch |
| | on cotton | on cotton |
| | Δ1.0 | Δ1.9 |

| | |
|---|---|
| Low concentration enzyme mixture | CS-73 |
| Water hardness 24 FH | Locust bean |
| | gum on cotton |
| | Δ1.2 |
| Low concentration enzyme mixture | CS-73 |
| Water hardness 48 FH | Locust bean |
| | gum on cotton |
| | Δ1.7 |

The results show that under different wash conditions and on different types of stains, here illustrated with protein (EMPA164), starch (CS-28) and mannan (CS-73), as well as different types of textiles, here illustrated with cotton, the combination of Hi3 together with a mixture of stain removing enzymes was capable of increasing the stain removing effect of the enzymes as compared to when the Hi3 is not present.

### Example 25a

### Hi3 in Combination with an Anti-Redeposition Enzyme in Mini-TOM

This example sets out to test the effect of Hi3 (0.1 mg/L) on anti-redeposition on cotton textile. Hi3 was used in combination with an anti-redeposition enzyme at 0.005 nM. The tests were performed with the mini-TOM set-up described in the Material and Methods section with the indicated water hardness. The results are given as the remission value of the swatches after wash in the presence of cellulase and 0.1 mg/L Hi3 minus the remission value of the swatches after wash with cellulase and no added Hi3, i.e. the delta (Δ) remission; which reflects the enzyme anti-redeposition enhancing effect. The results are shown in Table 28.

TABLE 28

| Enzyme | Result |
|---|---|
| Cellulase | Δ5.4 ± 0.8 |
| Water hardness 48 FH | |

The results show that the combination of Hi3 with an anti-redeposition enzyme was capable of increasing the anti-redeposition effect of the enzyme as compared to when the Hi3 is not present.

### Example 26

### Hi1 in Combination with a Mixture of Stain Removing Enzymes in LOM

This example sets out to test the effect of Hi1 (0.1 mg/L or 0 mg/L) on stain removal from different pre-stained swatches. Hi1 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Hi1 minus the remission value of the swatches after wash with enzymes and no added Hi1, i.e. the delta (Δ) remission; which reflects the enzyme detergency enhancing effect. The results are shown in Table 29.

TABLE 29

| Enzyme mixture | Stain | | |
|---|---|---|---|
| High concentration | CS-28 | Wfk10D | CS-73 |
| enzyme mixture | Rice starch | Sebum on | Locust bean |
| Water hardness 24 FH | on cotton | cotton | gum on cotton |
| | Δ2.2 | Δ1.4 | Δ2.8 |

| | |
|---|---|
| Low concentration | CS-73 |
| enzyme mixture | Locust bean |
| Water hardness 24 FH | gum on cotton |
| | Δ3.2 |
| Low concentration | CS-28 |
| enzyme mixture | Rice starch |
| Water hardness 48 FH | on cotton |
| | Δ4.4 |

The results show that under different wash conditions and on different types of stains, here illustrated with starch (CS-28), fats and oil (wfk10D) and mannan (CS-73), as well as different types of textiles, here illustrated with cotton, the combination of Hi1 together with a mixture of stain removing enzymes was capable of increasing the stain removing effect of the enzymes as compared to when the Hi1 is not present.

### Example 26a

### Hit in Combination with an Anti-Redeposition Enzyme in Mini-TOM

This example sets out to test the effect of Hi1 (0.1 mg/L) on anti-redeposition on cotton textile. Hi1 was used in combination with an anti-redeposition enzyme at 0.005 nM. The tests were performed with the mini-TOM set-up described in the Material and Methods section with the indicated water hardness. The results are given as the remission value of the swatches after wash in the presence of cellulase and 0.1 mg/L Hi1 minus the remission value of the swatches after wash with cellulase and no added Hi1, i.e. the delta (Δ) remission; which reflects the enzyme anti-redeposition enhancing effect. The results are shown in Table 30.

TABLE 30

| Enzyme | Result |
|---|---|
| Cellulase | Δ8.8 ± 1.2 |
| Water hardness 24 FH | |
| Cellulase | Δ8.0 ± 1.4 |
| Water hardness 48 FH | |

The results show that the combination of Hi1 with an anti-redeposition enzyme was capable of increasing the anti-redeposition effect of the enzyme as compared to when the Hi1 is not present.

### Example 27

### Pp1 in Combination with a Mixture of Stain Removing Enzymes in LOM

This example sets out to test the effect of Pp1 (0.1 mg/L or 0 mg/L) on stain removal from different pre-stained swatches. Pp1 was used in combination with a mixture of stain removing enzymes (protease, amylase, lipase, mannanase). The tests were performed with a low enzyme concentration (LOM method A), as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Pp1 minus the remission value of the swatches after wash with enzymes and no added Pp1, i.e. the delta (Δ) remission; which reflects the enzyme detergency enhancing effect. The results are shown in Table 31.

TABLE 31

| | |
|---|---|
| Enzyme mixture | Stain |

| | | |
|---|---|---|
| Low concentration enzyme mixture | CS-28 | CS-73 |
| Water hardness 24 FH | Rice starch | Locust bean |
| | on cotton | gum on cotton |
| | Δ1.0 | Δ1.0 |

| | |
|---|---|
| Low concentration enzyme mixture | EMPA164 |
| Water hardness 48 FH | Grass |
| | on cotton |
| | Δ1.4 |

The results show that under different wash conditions and on different types of stains, here illustrated with protein (EMPA164), starch (CS-28), and mannan (CS-73), as well as different types of textiles, here illustrated with cotton the combination of Pp1 together with a mixture of stain removing enzymes was capable of increasing the stain removing effect of the enzymes as compared to when the Pp1 is not present.

### Example 28

### Vt1 in Combination with a Mixture of Stain Removing Enzymes in LOM

This example sets out to test the effect of Vt1 (0.1 mg/L or 0 mg/L) on stain removal from different pre-stained swatches. Vt1 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Vt1 minus the remission value of the swatches after wash with enzymes and no added Vt1, i.e. the delta (Δ) remission; which reflects the enzyme detergency enhancing effect. The results are shown in Table 32.

TABLE 32

| | |
|---|---|
| Enzyme mixture | Stain |

| | | |
|---|---|---|
| Low concentration enzyme mixture | CS-28 | CS-73 |
| Water hardness 48 FH | Rice starch | Locust bean |
| | on cotton | gum on cotton |
| | Δ1.2 | Δ3.2 |

The results show that under different wash conditions and on different types of stains, here illustrated with starch (CS-28) and mannan (CS-73), as well as different types of textiles, here illustrated with cotton, the combination of Vt1 together with a mixture of stain removing enzymes was capable of increasing the stain removing effect of the enzymes as compared to when the Vt1 is not present.

### Example 29

### Vt2 in Combination with a Mixture of Stain Removing Enzymes in LOM

This example sets out to test the effect of Vt2 (0.1 mg/L or 0 mg/L) on stain removal from different pre-stained swatches. Vt2 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Vt2 minus the remission value of the swatches after wash with enzymes and no added Vt2, i.e. the delta (Δ) remission; which reflects the enzyme detergency enhancing effect. The results are shown in Table 33.

TABLE 33

| | |
|---|---|
| Enzyme mixture | Stain |

| High concentration enzyme mixture | | CS-28 |
|---|---|---|
| Water hardness 24 FH | | Rice starch |
| | | on cotton |
| | | Δ2.7 |
| Low concentration enzyme mixture | CS-06 | CS-28 |
| Water hardness 24 FH | Salad dressing | Rice starch |
| | on cotton | on cotton |
| | Δ1.1 | Δ1.6 |
| Low concentration enzyme mixture | CS-73 | CS-28 |
| Water hardness 48 FH | Locust bean | Rice starch |
| | gum on cotton | on cotton |
| | Δ1.2 | Δ1.9 |

The results show that under different wash conditions and on different types of stains, here illustrated with starch (CS-06 and CS-28) and mannan (CS-73 and CS-06), as well as different types of textiles, here illustrated with cotton, the combination of Vt2 together with a mixture of stain removing enzymes was capable of increasing the stain removing effect of the enzymes as compared to when the Vt2 is not present.

### Example 30

### Cg1 in Combination with a Mixture of Stain Removing Enzymes in LOM

This example sets out to test the effect of Cg1 (0.1 mg/L or 0 mg/L) on stain removal from different pre-stained swatches. Cg1 was used in combination with a mixture of stain removing enzymes (protease, amylase, lipase, mannanase). The tests were performed with a low enzyme concentration (LOM method A) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L Cg1 minus the remission value of the swatches after wash with enzymes and no added Cg1, i.e. the delta (Δ) remission; which reflects the enzyme detergency enhancing effect. The results are shown in Table 34.

TABLE 34

| Enzyme mixture | Stain |
|---|---|
| Low concentration enzyme mixture | CS-28 |
| Water hardness 24 FH | Rice starch on cotton |
| | Δ1.9 |

The results show that under different wash conditions and on different types of stains, here illustrated with starch (CS-28), as well as different types of textiles, here illustrated with cotton, the combination of Cg1 together with a mixture of stain removing enzymes was capable of increasing the stain removing effect of the enzymes as compared to when the Cg1 is not present.

### Example 31

### At4 in Combination with a Mixture of Stain Removing Enzymes in LOM

This example sets out to test the effect of At4 (0.1 mg/L or 0 mg/L) on stain removal from different pre-stained swatches. At4 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L At4 minus the remission value of the swatches after wash with enzymes and no added At4, i.e. the delta (Δ) remission; which reflects the enzyme detergency enhancing effect. The results are shown in Table 35.

TABLE 35

| Enzyme mixture | Stain | | |
|---|---|---|---|
| High concentration | | PC-10 | CS-28 |
| enzyme mixture | | Oil/milk on | Rice starch |
| Water hardness 24 FH | | polyester- | on |
| | | cotton | cotton |
| | | Δ1.1 | Δ2.8 |
| Low concentration | EMPA164 | PC-05 | CS-28 |
| enzyme mixture | Grass | Blood/milk/ink | Rice starch |
| Water hardness 24 FH | on | on polyester- | on |
| | cotton | cotton | cotton |
| | Δ1.0 | Δ1.5 | Δ2.7 |

The results show that under different wash conditions and on different types of stains, here illustrated with protein (EMPA164, PC-05 and PC-10) and starch (CS-28), as well as different types of textiles, here illustrated with cotton and polyester/cotton, the combination of At4 together with a mixture of stain removing enzymes was capable of increasing the stain removing effect of the enzymes as compared to when the At4 is not present.

### Example 32

### At3 in Combination with a Mixture of Stain Removing Enzymes in LOM

This example sets out to test the effect of At3 (0.1 mg/L or 0 mg/L) on stain removal from different pre-stained swatches. At3 was used in combination with a mixture of stain removing enzymes (protease, amylase, lipase, mannanase). The tests were performed with a low enzyme concentration (LOM method A) or high enzyme concentration (LOM method B) as indicated in Table 6 in Material and Methods section at the indicated water hardness and using the stains number 1-6, 8 and 11-14 as described in Table 5. The results are given as the remission value of the swatches after wash in the presence of enzymes and 0.1 mg/L At3 minus the remission value of the swatches after wash with enzymes and no added At3, i.e. the delta (Δ) remission; which reflects the enzyme detergency enhancing effect. The results are shown in Table 36.

TABLE 36

| | |
|---|---|
| Enzyme mixture | Stain |

| | | |
|---|---|---|
| High concentration enzyme mixture | EMPA164 | CS-28 |
| Water hardness 24 FH | Grass | Rice starch |
| | on cotton | on cotton |
| | Δ1.3 | Δ2.8 |
| Low concentration enzyme mixture | EMPA117 | CS-28 |
| Water hardness 24 FH | Blood/milk/ink | Rice starch |
| | on polyester- | on |
| | cotton | cotton |
| | Δ1.5 | Δ2.7 |

The results show that under different wash conditions and on different types of stains, here illustrated with protein (EMPA117 and EMPA164) and starch (CS-28), as well as different types of textiles, here illustrated with cotton and polyester/cotton, the combination of At3 together with a mixture of stain removing enzymes was capable of increasing the stain removing effect of the enzymes as compared to when the At3 is not present.

### Example 33

### Pyrgallol Activity Assay

In a 50 mM Na-acetate buffer solution, pyrogallol

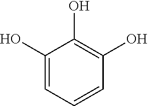

slowly changes color from colorless to yellow/brown. This color shift is accelerated by adding a GH61 polypeptide.

The assay was performed as follows:

GH61 polypeptide purified to electrophoretic homogeneity in a buffer without chelating agents (0.0125-0.1 mg/mL) was incubated with Pyrogallol (0.2% w/v), CaCl₂ and 50 mM Sodium acetate buffer, pH 5.0. Corresponding blanks were mixed by replacing GH61 with 50 mM Sodium acetate buffer, pH 5.0.

The samples were incubated for 48 hours in a thermo-mixer, 40° C., 850 rpm. After incubation the samples were centrifuged for 5 min, 13000 rpm in a table top centrifuge. 200 μL from each sample was carefully transferred to a 96-well microtitre plate by pipetting. Absorbance was measured at 440 nm using PowerWave instrument (Bio-Tek Instruments, Inc., US).

Activity was expressed as absorbance units after subtracting the corresponding blank.

The following results have been obtained using the activity assay described above.

A dose response experiment was conducted using Nc1 in the rage of 0.0125 to 0.1 mg/mL.

| GH61 (mg/mL) | ΔA440 |
|---|---|
| 0.0125 | 0.04 |
| 0.025 | 0.08 |
| 0.05 | 0.16 |
| 0.1 | 0.34 |

These results show a linear dose response correlation in the given range.

Three different GH61 polypeptides were tested for pyrogallol activity.

| GH61 | ΔA440 |
|---|---|
| Nc1 (0.1 mg/ml) | 0.38 |
| Ta1 (0.1 mg/ml) | 0.65 |
| Tt5 (0.1 mg/ml) | 0.45 |

These results show that GH61 polypeptide activity can be tested using pyrogallol as substrate.

In order to use this assay as an indicator of GH61 activity it is important that the GH61 polypeptides tested in the assay are purified in order to rule out interference from other non-GH61 components.

1-37. (canceled)

38. A method for enhancing the enzyme detergency benefit of one or more enzymes in a cleaning or textile care process comprising the steps:
a) combining one or more glycoside hydrolase family 61 polypeptide(s) with said enzymes; and
b) performing the cleaning or textile care process.

39. The method of claim 38, wherein the cleaning or textile care process is performed in the presence of a detergent.

40. The method of claim 38, where the detergency benefit enhancement is related to at least one of the following:
a) stain removal;
b) anti-redeposition;
c) whitening; or
d) a textile care benefit selected from the group consisting of
i) softening;
ii) prevention or reduction of pilling;
iii) colour clarification; and
iv) dye transfer inhibition.

41. The method of claim 38, wherein the enzyme is selected from the group consisting of proteases, cellulases, hemicellulases, lipases, cutinases, amylases, and pectinases.

42. The method of claim 38, wherein the enzyme is selected from the group consisting of metalloprotease, serine protease, triacylglycerol lipase, phospholipase A2, phospholipase A1, endoglucanases, xyloglucanases, alpha-amylases, pectate lyase, xylanases, and mannanases or mixtures thereof.

43. The method of claim 38, wherein the enzyme detergency benefit of the enzyme(s) is enhanced by at least 1 delta remission unit when the enzyme(s) is combined with a glycosyl hydrolase family 61 polypeptide as compared to when the enzyme(s) is used without the glycosyl hydrolase family 61 polypeptide when the assessment is performed as described in the Materials and Method section using Laundrometer set-up A at a water hardness of 24° FH for stain removal benefits or using the Small scale anti-redeposition washing method for anti-redeposition benefits.

44. The method of claim 38, wherein the enzyme is a stain removing enzyme.

45. The method of claim 44, wherein the stain removing enzyme(s) is selected from the group consisting of proteases, alpha-amylases, lipases and mannanases.

46. The method of claim 44, wherein the enhanced enzyme detergency benefit is assessed as the enhanced removal of a stain selected form the group consisting of protein containing stains, starch containing stains, fat and/or oil containing stains and mannan containing stains.

47. The method of claim 38, wherein the enzyme is an anti-redeposition enzyme.

48. The method of claim 47, wherein the anti-redeposition enzyme is cellulase or a xyloglucanase.

49. The method of claim 38, wherein the glycosyl hydrolase family 61 polypeptide is combined with at least two enzymes.

50. The method of claim 38, where the glycosyl hydrolase family 61 polypeptide is:
a) an isolated polypeptide comprising an amino acid sequence selected from the group consisting of:
Tt1 Amino acid residues 18 to 233 of SEQ ID NO:1;
Tt2 Amino acid residues 20 to 304 of SEQ ID NO:2;
At1 Amino acid residues 22 to 371 of SEQ ID NO:3;
Nc1 Amino acid residues 21 to 330 of SEQ ID NO:4;
Hi1 Amino acid residues 16 to 319 of SEQ ID NO:5;
Tt3 Amino acid residues 20 to 326 of SEQ ID NO:6;
Tt4 Amino acid residues 18 to 239 of SEQ ID NO:7;
Tt5 Amino acid residues 19 to 226 of SEQ ID NO:8;
Pp1 Amino acid residues 21 to 225 of SEQ ID NO:9;
Hi2 Amino acid residues 20 to 298 of SEQ ID NO:10;
Vt1 Amino acid residues 18 to 246 of SEQ ID NO:11;
Vt2 Amino acid residues 17 to 234 of SEQ ID NO:12;
At2 Amino acid residues 17 to 238 of SEQ ID NO:13;
Cg1 Amino acid residues 21 to 259 of SEQ ID NO:14;
Ta1 Amino acid residues 22 to 249 of SEQ ID NO:15;
Hi3 Amino acid residues 20 to 296 of SEQ ID NO:16;
At3 Amino acid residues 20 to 248 of SEQ ID NO:17; and
At4 Amino acid residues 21 to 302 of SEQ ID NO:18; or
b) an isolated polypeptide comprising an amino acid sequence which has at least 70% identity to one of the amino acid sequences in a); or
c) a functional fragment of a) or b).

51. The method of claim 38, wherein the cleaning process is a laundry process or hard surface cleaning process.

52. The method of claim 38, wherein the cleaning process is conducted in a water hardness below 100° FH.

53. A detergent composition comprising at least one enzyme and a glycosyl hydrolase family 61 polypeptide, wherein the enzyme detergency benefit of said detergent is enhanced by at least 1 delta remission unit as compared to a detergent without the glycosyl hydrolase family 61 polypeptide when the assessment is performed as described in the Materials and Method section using Laundrometer set-up A at a water hardness of 24° FH for stain removal benefits or using the Small scale anti-redeposition washing method for anti-redeposition benefits.

54. The detergent composition of claim 53, wherein the enzyme is selected from the group consisting of proteases, cellulases, hemicellulases, lipases, cutinases, amylases, and pectinases, or mixtures thereof.

55. The detergent composition of claim 53, wherein the enzyme is selected from the group consisting of metalloprotease, serine protease, triacylglycerol lipase, phospholipase A2, phospholipase A1, endoglucanses, xyloglucanases, alpha-amylases, pectate lyase, xylanases, and mannanases or mixtures thereof.

56. The detergent composition of claim 53, wherein the enzyme is a stain removing enzyme selected from the group consisting of proteases, alpha-amylases, lipases and mannanases.

57. The detergent composition of claim 53, wherein the glycosyl hydrolase family 61 polypeptide is:
a) an isolated polypeptide comprising an amino acid sequence selected from the group consisting of:
At1 Amino acid residues 22 to 371 of SEQ ID NO:3;
Nc1 Amino acid residues 21 to 330 of SEQ ID NO:4;
Pp1 Amino acid residues 21 to 225 of SEQ ID NO:9;
Hi2 Amino acid residues 20 to 298 of SEQ ID NO:10;
Vt1 Amino acid residues 18 to 246 of SEQ ID NO:11;
Vt2 Amino acid residues 17 to 234 of SEQ ID NO:12;
At2 Amino acid residues 17 to 238 of SEQ ID NO:13;
Cg1 Amino acid residues 21 to 259 of SEQ ID NO:14;
Hi3 Amino acid residues 20 to 296 of SEQ ID NO:16,
At3 Amino acid residues 20 to 248 of SEQ ID NO:17, and
At4 Amino acid residues 21 to 302 of SEQ ID NO:18; or
b) an isolated polypeptide comprising an amino acid sequence which has at least 70% identity to one of the amino acid sequences in a); or
c) a functional fragment of a) or b).

58. The detergent composition of claim 53, where the composition is a liquid or a powder detergent comprising less than 40% by weight of surfactant.

59. An isolated glycosyl hydrolase family 61 polypeptide, selected from the group consisting of:
a) a polypeptide comprising an amino acid sequence from amino acid residues 21 to 225 of SEQ ID NO:9;
b) a polypeptide with at least 75% identity with the amino acid sequence from amino acid residues 21 to 225 of SEQ ID NO:9;
c) a polypeptide comprising an amino acid sequence from amino acid residues 20 to 298 of SEQ ID NO:10;
d) a polypeptide with at least 85% identity with the amino acid sequence from amino acid residues 20 to 298 of SEQ ID NO:10;
e) a polypeptide comprising an amino acid sequence from amino acid residues 18 to 246 of SEQ ID NO:11;
f) a polypeptide with at least 70% identity with the amino acid sequence from amino acid residues 18 to 246 of SEQ ID NO:11;
g) a polypeptide comprising an amino acid sequence from amino acid residues 17 to 234 of SEQ ID NO:12;
h) a polypeptide with at least 70% identity with the amino acid sequence from amino acid residues 17 to 234 of SEQ ID NO:12;
i) a polypeptide comprising an amino acid sequence from amino acid residues 21 to 259 of SEQ ID NO:14;
j) a polypeptide with at least 95% identity with the amino acid sequence from amino acid residues 21 to 259 of SEQ ID NO:14;
k) a polypeptide comprising an amino acid sequence from amino acid residues 20 to 296 of SEQ ID NO:16;
l) a polypeptide with at least 80% identity with the amino acid sequence from amino acid residues 20 to 296 of SEQ ID NO:16;
m) a polypeptide comprising an amino acid sequence from amino acid residues 20 to 248 of SEQ ID NO:17;
n) a polypeptide with at least 97% identity with the amino acid sequence from amino acid residues 20 to 248 of SEQ ID NO:17; and
o) a functional fragment of (a) to (n).

60. An isolated polynucleotide comprising a nucleotide sequence which encodes one of the polypeptides of claim 59.

61. A method of washing and/or cleaning comprising treating a stained textile with a washing solution containing a detergent composition of claim 53.

62. The method of claim 61, wherein a detergent is dosed in an amount that is at least 5% by weight lower than a corresponding detergent without GH61 polypeptides and where at least the same detergency benefit is obtained with the reduced dose of the GH61 polypeptide-containing detergent when compared to the corresponding detergent without GH61 polypeptides.


SEQUENCE LISTING (57 sequences)

SEQ ID NO: 1 - protein, 233 aa, Thielavia terrestris
MKLTTSVALLAAAGAQAHYTFPQTDINGQLSGEWVTIRETTNHYSHGPVTDVTSDQIRCYELNPGTPAPQIATVQAGGTVTFTVDPSIQHPGPLQFYMAKAPSGQTAATFQGTGNVWFKIYEDGPSGLGTSNITWPSSGKTEVSVKIPSCIAPGDYLLRVEHIALHSASTVGGAQFYLACAQLTVTGGTGTLNTGELVAFPGAYSATDPGILFQLYWPIPTSYTNPGPAPVSC

SEQ ID NO: 2 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 3 - protein, 371 aa, Aspergillus terreus
MHYLHSASLIAPLLAATKVAAHGHVTNVVVNGVYYQGFDIGSFPYMADPPKVAAWTTPNTGNGFISPEQYGSADIICHENATNAQAHIPIKAGDRINLQWTAWPESHHGPVIDYLARCDGSCSTVDKTSLEFFKIDGVGLVDDSDVPGVWGDDQLIKNNNSWMVEIPKSIAPGNYVLRHELIALHSAQTEGGAQNYPQCFNLQVTGSGTDQPSGVVGTKLYSESDPGIVVNIYSSLSSYTVPGPTMYSGAASITQTSSAVTSTGTAVTGTGGASQAAVAVPTSEVTTLPVQVPTTLMTSAATPVSSSAIPSSSSIVPSTSPTSSPSAGTYGTQKLYGQCGGEGWVGPTACGQGATCRAYNRYYHQCVSSTN

SEQ ID NO: 4 - protein, 330 aa, Neurospora crassa
MRSTLVTGLIAGLLSQQAAAHATFQALWVDGADYGSQCARVPPSNSPVTDVTSNAMRCNTGTSPVAKKCPVKAGSTVTVEMHQQANDRSCSSEAIGGAHYGPVLVYMSKVSDAASADGSSGWFKIFEDTWAKKPSSSSGDDDFWGVKDLNSCCGKMQVKIPSDIPAGDYLLRAEVIALHTAASAGGAQLYMTCYQISVTGGGSATPATVSFPGAYKSSDPGILVDIHSAMSTYVAPGPAVYSGGSSKKAGSGCVGCESTCKVGSGPTGTASAVPVASTSAAAGGGGGGGSGGCSVAKYQQCGGTGYTGCTSCASGSTCSAVSPPYYSQCV

SEQ ID NO: 5 - protein, 319 aa, Humicola insolens
MRPFSLVALATAVSGHAIFQRVSVNGVDQGQLKGVRAPSSNYPIENVNHPDFACNTNIRHRDGTVIKIPAGATVGAWWQHEIGGPSFPGDPDNPIAASHKGPIQVYLAKVDNAATASPNGLRWFKIAEKGLSGGVWAVDEMIRNNGWHYFTMPQCIAPGHYLMRVELLALHSASFPGGAQFYMECAQIEVTGSGNFSPSETVSFPGAYPANHPGIVVSIYDAQGNANNGGREYQIPGPRPITCSGGGSNNGGGNNNGGGNNNGGGGNNNGGGNNNGGGNTGGGSAPLWGQCGGNGYSGPTTCAEGTCKKQNDWYSQCTP

SEQ ID NO: 6 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 7 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 8 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 9 - protein, 225 aa, Poronia punctata
MKTFARIAAVSAIAVNSASAHYIFQTFTAGDTQFDTFQYIRENSNYNSPVTDLTSNDLRCNVGASGASTETIEMTAGESFTFTLDTPVYHQGATSIYMSKAPGDVSEYEGDGEWFKIKDIGPDFSGGEATWDLSDSYSGEIPSCIEDGEYLLRIQQLAIHNPWPSGIPQFYISCAQISVTGGSGSAAPETALIPGFITEEDPGYTANIYSDFTSYEIPGPAPLSC

SEQ ID NO: 10 - protein, 298 aa, Humicola insolens
MHVQSLLAGALALAPSASAHFLFPHLMLNGVRTGAYEYVREHDFGFMPHNNDWINSPDFRCNEGSWRHRREPKTAVVTAGVDVVGFNLHLDFDLYHPGPVTIYLSRAPGDVRDYDGSGDWFKVYQLGTRQPFNGTDEGWATWKMKNWQFRLPREIPAGEYLMRIEQMSVHPPYRQKEWYVQCAHLKINSNYNGPAPGPTIKIPGGYKISDPAIQYDQWAQPPPTYAPMPGPALWPNNNPQQGNPNQGGNNGGGNQGGGNGGCTVPKWGQCGGQGYSGCRNCESGSTCRAQNDWYSQCL

SEQ ID NO: 11 - protein, 246 aa, Verticillium tenerum
MKFTAVSALGFAALAQGHAIFQVISVNGLEYPSLSGLRAPNQNNPVENVNSNDLTCGLVATTSTDVVEAAGGDTIGAWYQHVIGGAQFPGDPDNPIAASHKGPITAWLAKVDDAATASHQGLSWFKIAEDNFDTSSGVWGVDNLLNQDGWAYFELPDCIAPGDYLLRVELLALHSAYSSGGAQFYSSCANLRVTSGGSFEPSQTVSIPGVYQQNDPSIQIMIYGTSGNPDNDFQEYLAPGPRPITC

SEQ ID NO: 12 - protein, 234 aa, Verticillium tenerum
MKYSLSLLASASLALGHATFQQLWVDGVDQDTACARLPQSNNPVESVTSNDLTCNVGGRSAGHSGLCQVPAGSTVTVEMHEQPNERDCGRPAIGGNHYGPVLVYMSAVSDATSADGSGSWFKVGEYGYEDGIWGTDLLNENCGHFDFVVPAGLPSGDYLVRAEAIALHVAGSPGGAQFYMTCYQVSVTGGGSASVPSGVSFPGAYSATDPGILINIYTGDISNYQIPGPAVVNV

SEQ ID NO: 13 - protein, 238 aa, Aspergillus terreus
MKYALALASLVAAVSAHYTFDVLVVDGQETSSWQYIRENTRAEKYMPTKFINSPSITPLDSDFTCNEGANTNAGKTEVATVAAGSELAMKLAYGARIQHPGPAQVYMSKAPGNVKDYDGSGDWFKIYQDTVCTPGVELTEGGWCSWDKDRISFTIPASTPPGQYLVRAEHIALHGAHGGEAEFYYSCAQVEVTGSGSGEPSPVVKIPGVYAQDDEAVNFSVWGATDYPLIPGPEVWSG

SEQ ID NO: 14 - protein, 259 aa, Chaetomium globosum
MAPLTSAALILGSLASLVAGHGYLKSITVNGENYLAWQVGQDDYVTPTPVRYARKLADNGPVPDFTSNNITCGAGGNIPAEGVIELKAGDTVSLNWDQWGSSHSGPVMTYLAHCTNDDCKTFSGDTGAVWVKIEQLAYNAAGNPPWASDLLREQGAKWRVTIPPSLAPGEYLLRHEILGLHVAGVRMGAQFYPSCTQIRVTEGGSAALPAGIALPGAYDPDDAGILTELWRINQGQIPYTAPGGDVWGEAAPNANRDGP

SEQ ID NO: 15 - protein, 249 aa, Thermoascus aurantiaticus
MSFSKIIATAGVLASASLVAGHGFVQNIVIDGKNYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 16 - protein, 296 aa, Humicola insolens
MKGLLSIAALSLAVGEASAHYIFQQLSTGGTKHPMWKYIRQHTNYNSPVIDLDSNDLHCNVGARGAGTETVTVAAGSSLTFHLDTPIYHQGPVSVYMSKAPGSVSDYDGSGGWFKIQDWGPTFTGGGATWKLDDSYTFNIPSCIPDGEYLVRIQSLGIHNPWPAGIPQFYISCAQVRVTGGGNANPGPQVSIPGAFKETDPGYTANIYNNFRSYTVPGPSVFTCSGNSGGGSNPSNPNPPTPTTFITQVPNPTPVSPPTCTVAKWGQCGGQGYSGCTNCEAGSTCRQQNAYYSQCI

SEQ ID NO: 17 - protein, 248 aa, Aspergillus terreus
MSLSKIATGILASATLVAGHGYVSGIVADGKYYSGYLVDKYSYMDDPPETIGWSTTATDLGFVDGTGYDTVDIACHKGSAPGALTATVPAGSKIEMQWNTWPESHHGPVLNYLAPCNGDCAQADKSSLEFFKIEAEGLIDGSSPPGEWATDELISNNNTAVVTIPASIASGNYVLRHEIIGLHSAGNLNGAQNYPQCINIEITGGGSTKPSGVSATTFYKNTDPGIKFDIYSDLSGGYPMPGPALFDA

SEQ ID NO: 18 - protein, 302 aa, Aspergillus terreus
MKYLPTLFAATAALAPSASAHYIFSKLVLNGEVSEDWQYIRETTRSECYMPTKFTNTFDNLTPNDSDFRCNLGSFSNAAKTEVAEVAAGDTIGMKLFYDTSIAHPGPGQVFMSKAPSGNVQEYEGDGEWFKIWEKTLCNENGDLTKDAWCTYGMSQFEFQIPENTPAGEYLVRAEHVGLHGAQANEAEFFYSCAQIKVTGSGNGSPSQTYKIPGLYNDNMKLFNGLNLWVDSADAIETDILDTPVGDDVWNGSGSSSSSSSSSSSGSASSSAAAAASSTPSYSGYSSYGQQYSQQYSQCGAN

SEQ ID NO: 19 - DNA, 946 bp, Poronia punctata
ggccattacggccggggaggttcaaggatagtagcgcaaatcacaaagtcgaacgaaaaccgaaaatgaagacctttgcccgcatcgccgccgtctcggccatcgctgttaacagcgcttcggcccattacatcttccagactttcactgctggagatactcagttcgacaccttccagtacatccgtgagaactccaactacaactctcccgtcacggacctcacctccaacgacctccgctgcaatgtcggcgccagcggtgccagcaccgagaccattgagatgacggccggtgaatccttcactttcaccctagacactcccgtctaccaccagggtgccacctccatctacatgtccaaggcccccggcgacgtgtccgagtacgaaggtgacggagaatggttcaagatcaaggacatcggcccagacttctccggcggcgaagccacttgggatctatccgatagctactctggcgagatccccagctgcatcgaggacggcgaatatcttctccgcatccagcagctcgccatccacaacccctggccttcgggaattccccagttctacatctcttgcgcccagatcagtgtcaccggtggctccggctccgctgcccccgagactgctctgatccccggcttcatcactgaggaggatcccggctacactgccaacatctacagcgacttcacctcttacgagatcccaggccccgctcccctgtcctgctaaattacacatcaatgaatgaatgcgtgggaagctctgacaacctctcttccctaaaacgcaaaatcaatagggggaagaacctatcgtggtggttggtttaagagaagcagtttaggtcaacaccataactggcctgattcttttacttgtatatatttttaccatacctagtccaataacataaacaaagacttgatacatat

SEQ ID NO: 20 - DNA, 963 bp, Humicola insolens
tcttcttctaatcctctatatacacaactgaaattcaccatgcacgtccagtctctccttgccggagcgctcgctctggcgccgtcggcgtctgctcacttcctcttcccgcacctgatgctgaacggtgtccgcacgggggcctacgagtatgtccgggagcacgacttcggcttcatgccgcacaacaacgactggatcaactcccccgacttccgctgcaatgaggggtcctggcgtcatcgccgggagcccaagactgccgtggtcaccgccggcgttgatgtcgtaggcttcaacttgcacctggattttgacctgtaccatccggggcccgtaactatctacctctcccgcgcccccggcgacgtgcgcgactacgacggctccggcgactggttcaaggtctaccagctgggcacgcgccagcccttcaacggcaccgacgagggctgggctacctggaagatgaagaactggcagttccgcctgccccgcgagatccccgcgggcgagtacctgatgcgcatcgagcagatgagcgtgcaccctccttaccgccagaaggagtggtacgtgcagtgcgcccacctcaagatcaacagcaactacaacggccccgcacccggcccgactatcaagattcccggaggatacaagatcagcgatcccgcgattcagtatgaccagtgggcgcagccgccgccgacgtacgcgcccatgccgggaccggcgctgtggcccaacaacaatcctcagcagggcaacccgaatcagggcggaaataacggcggtggtaaccagggcggcggcaatggtggctgcaccgttccgaagtggggccaatgcggtggtcagggctacagcgggtgcaggaactgcgagtctggctcgacatgccgtgcccagaacgactggtactcgcagtgcctgtaaggtctggacgccgaagcgtgcggccgc

SEQ ID NO: 21 - DNA, 936 bp, Verticillium tenerum
catcaactgcaccacaagcagaagctgaaagttcgtctcctactacaccaacagcagccatgaagttcactgccgtctcagctctcggctttgccgccctcgcccagggccatgccatcttccaggtcatctccgtcaacggcctcgagtacccttccctctccggcctccgcgccccgaaccagaacaaccccgtcgagaacgtcaactccaacgacctgacctgcggcctcgtcgccaccacctccaccgacgtcgtcgaggctgccggcggcgacaccatcggcgcctggtaccagcacgtcatcggcggcgcccagttccccggcgacccagacaacccaatcgcggcctcccacaagggccccatcaccgcctggctggccaaggtcgatgatgccgccacggccagccaccagggcctctcctggttcaagatcgccgaagacaacttcgacacgtcctccggagtctggggcgtcgacaaccttctcaaccaggacggttgggcgtacttcgagctgcccgactgcatcgcccctggcgactacctgctgcgcgttgagctgctcgctctccactcggcttattcctctggaggcgctcagttctactcgtcttgcgccaacctccgcgttaccagcggcggcagcttcgagccgagccagaccgtttctatcccgggtgtttaccagcagaacgacccgtctatccagatcatgatctacggtacttctggcaacccggacaacgacttccaggagtacctcgctcccggaccccgtcccattacctgctaggctagtgatgagacttgaaaagaatattggcggaataggagactgagttacgagacttgagcccgatgaatatgggaggattgtacatagaatgaaggcatccgtatataaatgaaatgtaacgactgaacccctg

SEQ ID NO: 22 - DNA, 975 bp, Verticillium tenerum
gcacgaggccgctttcgtcttcttcacttcatcacatcatatccttcaagctcacacatcatgaagtactcgctctctctactggcttccgccagcctggccctcggccacgccaccttccagcagctttgggttgacggggtcgaccaggacacggcctgcgctcgcctgccccagagcaacaaccccgtcgagagcgtcacctccaacgacctgacctgcaacgtcggcggcaggtccgccggccactctggcctctgccaggtccctgccggctcgaccgtcaccgtcgagatgcacgagcagcccaacgagcgcgactgcggccgccccgccatcggcggcaaccactacggccccgtcctggtctacatgtccgccgtcagcgacgccacctcggccgacggctccggctcttggttcaaggtcggcgagtacggctacgaggacggcatctggggcaccgacctcctcaacgagaactgcggccacttcgactttgtcgtccccgccggcctgccctccggcgactacctcgtccgcgccgaggccattgctctccacgtcgccggctctcctggcggtgcgcagttctacatgacttgctaccaagtctccgtcactggcggcggtagtgcctccgtcccctctggcgtctccttccccggtgcctactccgcgaccgaccctggtattctcatcaacatctacaccggcgacatctccaactaccagattcccggccctgctgtggtcaacgtctaggctatcccgttttaggtctttggagcaacccctggcaagtttgtactaagatgggatcagataggctattcgcgtgaggtggaaggcctcggtagttgaggttggggagcatcttgggatattaaagcctaggctatgatattatttgcatagcgagaaatgaaacaagacaatcaactttgaattaaaaaaaaaaaaaaaaaaaaaaaa

SEQ ID NO: 23 - DNA, 777 bp, Chaetomium globosum
atggcacccttgacatccgcggccctgatcctgggcagccttgccagcctcgttgctggccatggctacctcaagagcatcaccgtcaacggcgagaactacctcgcatggcaggttggccaggacgactatgtcaccccgacgccggttcgatacgcccgcaagcttgccgacaacggcccggttccggactttaccagcaacaacatcacatgtggcgcagggggcaacatccccgccgagggagtgattgagttgaaggcgggcgacactgtctccctcaactgggaccaatggggcagctctcacagcggtccggtcatgacctacctcgcgcactgcaccaacgacgactgcaagaccttctcgggcgacacgggcgccgtgtgggtcaagatcgagcagctggcctacaacgcggcgggcaaccctccctgggcgtccgacctgctgcgcgagcagggggccaagtggcgggtgacgatcccgccgtcgctggcgcccggcgagtacctgctccggcacgagatcctgggcctgcacgtggccggggtgcgcatgggcgcccagttctacccgagctgtacccagatccgcgttaccgagggcgggagcgcggcgctgccggcggggatcgcgctgccgggcgcgtatgacccggatgatgcgggcattttgaccgagttgtggaggattaaccagggccagatcccctacacggccccgggaggagatgtctggggtgaggcggcgcccaacgcgaacagggatggcccg

SEQ ID NO: 24 - DNA, 1175 bp, Humicola insolens
cacgtagccatcgctgctccaacgtttcaaccccagcccaggaactcatcctcgttgcaacggcacggtagctaggcaaccgacgacatcaaccaccatgaagggacttctcagcatcgccgccctttccctggcggttggtgaggcttcggcccactacatcttccagcagctctccacgggcggcaccaagcaccccatgtggaagtacatccgccagcacaccaactacaactctcccgtcatcgacctcgactccaatgacctccactgcaatgttggtgctcgtggcgctggaaccgagaccgtcacagtcgctgctggctcgagcttgaccttccacctcgacacccccatctaccaccagggccctgtgtcggtctacatgtccaaggctcccggctccgtgtcggactatgacggcagcggcggctggttcaagattcaggactggggcccgaccttcaccggcggcggcgccacctggaagctggatgactcctacaccttcaacatcccctcgtgcattcccgacggcgagtacctcgtccgcatccagtccctgggtatccacaacccctggccggcgggtatcccgcagttctacatctcgtgcgctcaggtgcgcgtcaccggcggtggcaacgccaacccgggcccgcaggtgtcgatcccgggcgccttcaaggagaccgaccccggctacactgccaacatctacaacaacttccgcagctacaccgtccccggcccgtccgtcttcacctgcagcggcaacagcggcggcggctccaaccccagcaaccccaaccccccgaccccaaccaccttcatcactcaggtccctaacccgacccccgtttctccgcccacctgcaccgtcgcgaagtggggccagtgcggtgggcagggctacagcggctgcaccaactgcgaggccggctcgacctgcaggcagcagaacgcttactactctcagtgcatctaaagagtcattagagggaaagagggaaacacgggaagcgttgggctagatgactcaggacatatgaagaacaaaggggggttctgcatctgcatggtctggggggaactagtagatgggaagaaagaggcatcttctcctcttatcccacatatttttaccttcgaaagtacatacttttgcttcaaaa

SEQ ID NO: 25 - DNA, 840 bp, Aspergillus terreus
atgtccctgtctaagattgctacgggcattcttgcctcggccaccctcgtggcgggccacggttacgtctctggaatcgttgccgatggcaaatagtaagtctgctgtatggaccgcatacatgcacaagtgaggctgacagtccaacagctactcgggttacctcgtcgacaagtattcctacatggacgacccgcccgagaccattggctggtccaccaccgccaccgacctcggcttcgtcgacggtactggttatgacactgtcgacattgcctgccataagggcagtgctcccggcgccttgactgccaccgtgcccgccggctcgaagatcgagatgcagtggaacacctggcctgagagccaccacggccccgtcctcaactacctcgccccttgcaatggcgactgcgcccaggcggacaagtcctctctggagttcttcaagatcgaggccgagggtctcatcgacggcagctctcctccgggcgaatgggccactgacgagctgatctccaacaacaacaccgccgtcgtcaccatcccggcttccattgccagcggaaactacgtcctccgccacgagatcatcggtcttcactccgccggcaacctcaacggtgctcagaactacccccagtgcatcaacattgagatcactggtggcggcagcaccaagccatctggtgtctctgccacgaccttctacaagaacaccgaccccggcatcaagttcgacatctactctgacctgagtggtggataccccatgcccggacctgccctgttcgatgcttaaatagaggcggtacctggagcatggcttacgaacacctg

SEQ ID NO: 26 - DNA, 1041 bp, Chaetomium globosum
atggcacccttgacatccgcggccctgatcctgggcagccttgccagcctcgttgctggccatggctacctcaagagcatcaccgtcaacggcgagaactacctcgcatggcaggttggccaggacgactatgtcaccccgacgccggttcgatacgcccgcaagcttgccgacaacggcccggttccggactttaccagcaacaacatcacgtatgcccaaactcaaggcccaatatgtgaggccactgctgattatcaagctagatgtggcgcagggggcaacatccccgccgagggagtgattgagttgaaggcgggcgacactgtgtatgagaaagcctcttcccgcaagtctcacggccatactgacgcaatcaacttcacagctccctcaactgggaccaatggggcagctctcacagcggtccggtcatgacgtgaggccctccccttcccaacttcactcaaccgccctcaccctaacaaaccactccctcaccccagctacctcgcgcactgcaccaacgacgactgcaagaccttctcgggcgacacgggcgccgtgtgggtcaagatcgagcagctggcctacaacgcggcgggcaaccctccctgggcgtccgacctgctgcgcgagcagggggccaagtggcgggtgacgatcccgccgtcgctggcgcccggcgagtacctgctccggcacgagatcctgggcctgcacgtggccggggtgcgcatgggcgcccagttctacccgagctgtacccagatccgcgttaccgagggcgggagcgcggcgctgccggcggggatcgcgctgccgggcgcgtatgacccggatgatgcgggcgtgagtgactggtttcttttttttctggccccgagatggggggtttcgagggtgtggttggctgacaatggttggtgatagattttgaccgagttgtggaggattaaccagggccagatcccctacacggccccgggaggagatgtctggggtgaggcggcgcccaacgcgaacagggatggcccgtag

SEQ ID NO: 27 - DNA, 1261 bp, Neurospora crassa
atgcggtccactcttgtcaccggcctcatcgccggcctactctcccaacaagccgccgcccacgccaccttccaagccctttgggtcgatggtgccgattatggctcgcaatgcgctcgcgtccctccttccaactcccccgtcaccgatgtgactagcaatgccatgaggtgtaacacgggaacttcgcccgttgcgaagaagtgccctgtcaaggcgggaagtacggtcactgttgagatgcaccaggtatgctgccattctcctgctctcgtcaaccgccggcttcgcccctgactgccttccatgcggagcaggttctcaagagttacctgcttttccctccctctcttctctttcctcttccctccgccacctcctttttttttttccatcccgcctgttcatgttcgcctcagtcacaccctcccgtaccgacgctgacctataagcagcaagcaaatgaccgctcctgttcctctgaagccatcggtggcgctcactacggtcccgtcctcgtgtatatgtccaaggtctccgacgccgcctccgccgacggttcctctggctggttcaagatctttgaggacacctgggccaagaagccctccagctcctcgggcgacgatgatttctggggcgtcaaagacctcaactcgtgctgcggcaagatgcaggtcaagatcccctcggacatccccgcgggtgactatctcctccgtgccgaggttatcgcgctccataccgccgcaagcgcgggaggtgcccagttgtacatgacctgctaccagatctccgttaccggtggtggctccgctaccccggcgactgtcagctttcctggtgcctacaagagctccgaccctggtatcctcgttgacatccacagtgccatgagcacctacgtcgcccccggaccggctgtgtactcgggtggaagctccaagaaggccggaagcggctgcgtgggctgcgagtctacttgcaaggttggctccggcccgactggaactgcttctgccgtccctgttgcgagcacgtcggcggctgctggtggtggaggcggtggtgggagcggtggctgcagcgttgcaaagtatcagcagtgtggtggaaccggctataccgggtgcacatcctgcgctgtgagtgtcctcctgtttaatggtcgagttgagggttggcttgctaatgtgactttatctagtccggatccacctgcagcgctgtctcacctccttattactcccagtgtgtctaa

SEQ ID NO: 28 - DNA, 900 bp, Neurospora crassa
atgcggtccactcttgtcaccggcctcatcgccggcctactctcccaacaagccgccgcccacgccaccttccaagccctttgggtcgatggtgccgattatggctcgcaatgcgctcgcgtccctccttccaactcccccgtcaccgatgtgactagcaatgccatgaggtgtaacacgggaacttcgcccgttgcgaagaagtgccctgtcaaggcgggaagtacggtcactgttgagatgcaccagcaagcaaatgaccgctcctgttcctctgaagccatcggtggcgctcactacggtcccgtcctcgtgtatatgtccaaggtctccgacgccgcctccgccgacggttcctctggctggttcaagatctttgaggacacctgggccaagaagccctccagctcctcgggcgacgatgatttctggggcgtcaaagacctcaactcgtgctgcggcaagatgcaggtcaagatcccctcggacatccccgcgggtgactatctcctccgtgccgaggttatcgcgctccataccgccgcaagcgcgggaggtgcccagttgtacatgacctgctaccagatctccgttaccggtggtggctccgctaccccggcgactgtcagctttcctggtgcctacaagagctccgaccctggtatcctcgttgacatccacagtgccatgagcacctacgtcgcccccggaccggctgtgtactcgggtggaagctccaagaaggccggaagcggctgcgtgggctgcgagtctacttgcaaggttggctccggcccgactggaactgcttctgccgtccctgttgcgagcacgtcggcggctgctggtggtggaggcggtggtgggagcggtggctgcagcgttgcaaagtatcagcag

SEQ ID NO: 29 - DNA, 840 bp, Aspergillus terreus; identical to SEQ ID NO: 25

SEQ ID NO: 30 - DNA, 1174 bp, Aspergillus terreus
atgcattacctgcactccgcttcgctgatcgcaccgctgctggcagctactaaggtggctgcacatggccatgtcactaacgtggtcgtcaatggagtctactatcaaggattcgacattgggagcttcccgtacatggccgaccctccaaaggtagcggcttggaccacgcccaatacgggcaacggcttcatctcgccagagcaatatggatctgccgatatcatctgccacgagaacgctaccaacgcgcaagcccacatccctatcaaggccggagaccgcatcaacctccaatggacggcatggcctgagtctcaccacggccctgtcatcgactatctggctcgctgcgacggcagctgctccaccgtcgacaaaacgagcctcgagttcttcaaaatcgacggtgtcggcctcgttgacgattcggatgtccctggcgtctggggcgatgaccagctcatcaagaacaacaacagctggatggtcgagattcccaagtccattgcgccaggaaactacgtgctccggcacgagttgattgccctacacagtgcacagaccgagggcggtgcgcagaactatccccagtgcttcaatcttcaggtcaccggctccgggacggatcagccatccggtgttgtcggaaccaagctgtactccgaatctgatccggggattgtggtcaacatctactcctctctttcttcctataccgtgcccggtccaaccatgtacagcggcgccgcctccatcactcagacctcgtcggcggtaacctcgactggcaccgccgtgaccgggacgggaggcgcaagccaggctgccgtggctgttcctacgtcggaggtgacgacactgccggtccaagtccccacgaccttgatgacgagcgccgccaccccagtgtcatcctctgcgatcccatcctcgtcgtcgatcgtgccttccacgtcccctacttcgtctccttccgcgggtacatatggcacgcagaagctgtatggacaatgcggtggtgaaggatgggttggtcccactgcctgtggacagggtgccacttgccgtgcttataaccggtactatcaccagtgtgtctcttccacgaattagttggcattgacaggaagccatggagtggtctgcatacagctgtttctacctgaggagc

SEQ ID NO: 31 - DNA, 1184 bp, Aspergillus terreus
atgcattacctgcactccgcttcgctgatcgcaccgctgctggcagctactaaggtggctgcacatggccatgtcactaacgtggtcgtcaatggagtctactatcaaggattcgacattgggagcttcccgtacatggccgaccctccaaaggtagcggcttggaccacgcccaatacgggcaacggcttcatctcgccagagcaatatggatctgccgatatcatctgccacgagaacgctaccaacgcgcaagcccacatccctatcaaggccggagaccgcatcaacctccaatggacggcatggcctgagtctcaccacggccctgtcatcgactatctggctcgctgcgacggcagctgctccaccgtcgacaaaacgagcctcgagttcttcaaaatcgacggtgtcggcctcgttgacgattcggatgtccctggcgtctggggcgatgaccagctcatcaagaacaacaacagctggatggtcgagattcccaagtccattgcgccaggaaactacgtgctccggcacgagttgattgccctacacagtgcacagaccgagggcggtgcgcagaactatccccagtgcttcaatcttcaggtcaccggctccgggacggatcagccatccggtgttgtcggaaccaagctgtactccgaatctgatccggggattgtggtcaacatctactcctctctttcttcctataccgtgcccggtccaaccatgtacagcggcgccgcctccatcactcagacctcgtcggcggtaacctcgactggcaccgccgtgaccgggacgggaggcgcaagccaggctgccgtggctgttcctacgtcggaggtgacgacactgccggtccaagtccccacgaccttgatgacgagcgccgccaccccagtgtcatcctctgcgatcccatcctcgtcgtcgatcgtgccttccacgtcccctacttcgtctccttccgcgggtacatatggcacgcagaagctgtatggacaatgcggtggtgaaggatgggttggtcccactgcctgtggacagggtgccacttgccgtgcttataaccggtactatcaccagtgtgtctcttccacgaattagttggcattgacaggaagccatggagtggtctgcatacagctgtttctacctgaggagccatgtatctg

SEQ ID NO: 32 - DNA, 717 bp, Aspergillus terreus
atgaagtacgcactcgctcttgcttctcttgttgccgctgttagcgcgcactacaccttcgatgtcctcgtcgtcgatggccaggagacttcgagctggcagtacatccgcgagaacacccgcgcggagaagtacatgccgaccaagttcatcaactcgccgtctatcactccgctagattccgacttcacctgcaacgaaggcgccaacaccaacgccggcaagaccgaggttgccaccgttgcggctggctccgagctggccatgaagctcgcttatggagcccgcatccagcatcccggtcccgcccaggtctacatgtcgaaagcccccggcaacgtcaaggactacgacggctctggcgactggttcaagatctaccaggacaccgtctgcacccctggcgtggagctgactgaaggaggctggtgctcctgggacaaggaccgcatcagctttaccattccggcctccacccctccgggccagtacctcgtccgtgctgagcatattgctctgcacggtgcccacggcggtgaggctgagttctattactcctgcgcccaggttgaggtcacgggctccggcagcggagagcccagtcctgtcgttaagatccccggcgtgtatgctcaggatgacgaggctgtcaacttcagcgtctggggtgccactgactatcccctcattcctggccctgaggtctggtctggttaa

SEQ ID NO: 33 - DNA, 830 bp, Aspergillus terreus
atgaagtacgcactcgctcttgcttctcttgttgccgctgttagcgcgcactacaccttcgatgtcctcgtcgtcgatggccaggagacttcgagctggcagtacatccgcgagaacacccgcgcggagaagtacatgccgaccaagttcatcaactcgccgtctatcactccgctagattccgacttcacctgcaacgaaggcgccaacaccaacgccggcaagaccgaggttgccaccgttgcggctggctccgagctggccatgaagctcgcttatggagcccgcatccagcatcccgtacgtaccagcctcttcactataacgaaacagaacgcaaactaattatctgcaatccagggtcccgcccaggtctacatgtcgaaagcccccggcaacgtcaaggactacgacggctctggcgactggttcaagatctaccaggacaccgtctgcacccctggcgtggagctgactgaaggaggctggtgctcctgggacaaggaccgcatcagctttaccattccggcctccacccctccgggccagtacctcgtccgtgctgagcatattgctctgcacggtgcccacggcggtgaggctgagttctattactcctgcgcccaggttgaggtcacgggctccggcagcggagagcccagtcctgtcgttaagatccccggcgtgtatgctcaggatgacgaggctgtcaacttcagcgtctggggtgccactgactatcccctcattcctggccctgaggtctggtctggttaaggacatattgtagttccttctcgatgatgatcgagtggttgctacaggcggaa

SEQ ID NO: 34 - DNA, 897 bp, Aspergillus terreus
atgaagtaccttcccactcttttcgcggctacagcggcgctggccccctctgcatctgcgcactacatcttcagcaagctcgttctgaatggtgaagtctccgaggactggcagtacattcgcgagactacccgcagcgaatgctacatgcccaccaagttcaccaacacctttgacaacctgacccccaacgacagcgacttccgctgcaacctgggctctttcagcaacgccgccaaaaccgaggttgcggaagtcgctgccggtgataccattggcatgaaacttttctacgatacctccattgcgcaccctggtcctggccaagttgtcatgtccaaggcgccgtccggcaacgtccaggagtacgagggcgatggcgaatggttcaagatctgggagaagaccctctgcaacgagaacggcgacctcaccaaggatgcctggtgcacttacggcatgtcgcagttcgagttccagatccccgagaacacccccgcgggcgagtaccttgttcgtgctgagcacgttggcctccatggcgcgcaggccaacgaagccgagttcttctacagctgcgctcaaatcaaggtcaccggcagcggcaacggttctcccagccagacatacaaaatccccggtctctacaacgacaacatgaagctcttcaacggtctcaacctctgggtcgactctgcggatgctatcgagactgatatcctggacacgcctgtcggagatgacgtttggaacggtagcggtagcagcagcagcagcagctcgtccggctctgcgagctccgccgccgccgccgcttccagcactcccagctattccggctattccagctacggccagcagtactcccagcagtactcccaatgcggtgccaactag

SEQ ID NO: 35 - DNA, 1043 bp, Aspergillus terreus
atgaagtaccttcccactcttttcgcggctacagcggcgctggccccctctgcatctgcgcactacatcttcagcaagctcgttctgaatggtgaagtctccgaggactggcagtacattcgcgagactacccgcagcgaatgctacatgcccaccaagttcaccaacacctttgacaacctgacccccaacgacagcgacttccgctgcaacctgggctctttcagcaacgccgccaaaaccgaggttgcggaagtcgctgccggtgataccattggcatgaaacttttctacgatacctccattgcgcaccctggtcctggccaagtttgtatgggcacccgttcgcctttttcgcgtcccatctgctaacgaagttttgcccagtcatgtccaaggcgccgtccggcaacgtccaggagtacgagggcgatggcgaatggttcaagatctgggagaagaccctctgcaacgagaacggcgacctcaccaaggatgcctggtgcacttacggcatgtcgcagttcgagttccagatccccgagaacacccccgcgggcgagtaccttgttcgtgctgagcacgttggcctccatggcgcgcaggccaacgaagccgagttcttctacaggtgagctgaatagctgaatcaatcctttgtgccggggagaaggaaagctgactgagttgtactgtacagctgcgctcaaatcaaggtcaccggcagcggcaacggttctcccagccagacatacaaaatccccggtctctacaacgacaacatgaagctcttcaacggtctcaacctctgggtcgactctgcggatgctatcgagactgatatcctggacacgcctgtcggagatgacgtttggaacggtagcggtagcagcagcagcagcagctcgtccggctctgcgagctccgccgccgccgccgcttccagcactcccagctattccggctattccagctacggccagcagtactcccagcagtactcccaatgcggtgccaactagacacggtccactgcgtctgcg

SEQ ID NO: 36 - DNA, 40 bp, artificial
  Primer sequence
acacaactggggatccaccatgaagtactcgctctctcta

SEQ ID NO: 37 - DNA, 35 bp, artificial
  Primer sequence
agatctcgagaagcttagacgttgaccacagcagg

SEQ ID NO: 38 - DNA, 39 bp, artificial
  Primer sequence
acacaactggggatccacccatgaagttcactgccgtct

SEQ ID NO: 39 - DNA, 35 bp, artificial
  Primer sequence
agatctcgagaagcttagcaggtaatgggacgggg

SEQ ID NO: 40 - DNA, 39 bp, artificial
  Primer sequence
acacaactggggatccaccatgaagacctttgcccgcat

SEQ ID NO: 41 - DNA, 34 bp, artificial
  Primer sequence
agatctcgagaagcttagcaggacaggggagcgg

SEQ ID NO: 42 - DNA, 29 bp, artificial
  Primer sequence
gcggaattcaccatgcacgtccagtctct

SEQ ID NO: 43 - DNA, 34 bp, artificial
  Primer sequence
atttgcggccgcacgcttcggcgtccagacctta

SEQ ID NO: 44 - DNA, 40 bp, artificial
  Primer sequence
acacaactggggatccacatgaagggacttctcagcatcg

SEQ ID NO: 45 - DNA, 43 bp, artificial
  Primer sequence
agatctcgagaagcttagatgcactgagagtagtaagcgttct

SEQ ID NO: 46 - DNA, 38 bp, artificial
  Primer sequence
acacaactggggatccaccatggcacccttgacatccg

SEQ ID NO: 47 - DNA, 34 bp, artificial
  Primer sequence
agatctcgagaagcttacgggccatccctgttcg

SEQ ID NO: 48 - DNA, 33 bp, artificial
  Primer sequence
taagaattcaccatgcattacctgcactccgct

SEQ ID NO: 49 - DNA, 34 bp, artificial
  Primer sequence
tatgcggccgcagatacatggctcctcaggtaga

SEQ ID NO: 50 - DNA, 30 bp, artificial
  Primer sequence
taagaattcatcatgaagtacgcactcgct

SEQ ID NO: 51 - DNA, 31 bp, artificial
  Primer sequence
tatgcggccgcttccgcctgtagcaaccact

SEQ ID NO: 52 - DNA, 33 bp, artificial
  Primer sequence
taagaattcacaatgtccctgtctaagattgct

SEQ ID NO: 53 - DNA, 31 bp, artificial
  Primer sequence
tatgcggccgcaggtgttcgtaagccatgct

SEQ ID NO: 54 - DNA, 36 bp, artificial
  Primer sequence
taagaattcaccatgaagtaccttcccactcttttc

SEQ ID NO: 55 - DNA, 27 bp, artificial
  Primer sequence
tatgcggccgcagacgcagtggaccgt

SEQ ID NO: 56 - DNA, 40 bp, artificial
  Primer sequence
acacaactggggatccaacatgcggtccactcttgtcacc

SEQ ID NO: 57 - DNA, 43 bp, artificial
  Primer sequence
agatctcgagaagcttagacacactgggagtaataaggaggtg